(19) United States
(12) Patent Application Publication
(10) Pub. No.: US 2002/0041898 A1
(43) Pub. Date: Apr. 11, 2002

(54) Novel targeted delivery systems for bioactive agents

(76) Inventors: Evan C. Unger, Tucson, AZ (US); Terry Onichi Matsunaga, Tucson, AZ (US); Varadarajan Ramaswami, Tucson, AZ (US); Marek J. Romanowski, Tucson, AZ (US)

Correspondence Address: David A. Cherry, Esq., WOODCOCK WASHBURN KURTZ, MACKIEWICZ & NORRIS LLP, One Liberty Place - 46th Floor, Philadelphia, PA, 19103, US

(21) Appl. No.: 09/912,609

(22) Filed: Jul. 25, 2001

Related U.S. Application Data
(63) Continuation-in-part of application No. 09/703,474, filed on Oct. 31, 2000.
Application No. 09/703,474 is a continuation-in-part of application No. 09/478,124, filed on Jan. 5, 2000.

(51) Int. Cl. A61K 9/14; A61K 39/395

(52) U.S. Cl. 424/486; 424/178.1

(57) ABSTRACT

Novel targeted delivery systems for bioactive agents. In preferred form, the delivery systems comprise, in combination with an effective amount of a bioactive agent, a targeted matrix comprising a polymer and a targeting ligand. Preferably, the targeting ligand is covalently associated with the polymer and the bioactive agent is associated non-covalently with the polymer. Also in preferred embodiments, the bioactive agent is substantially homogeneously dispersed throughout the matrix. The compositions are particularly suitable as delivery vehicles with bioactive agents that have limited water solubility.

### CROSS-REFERENCE TO RELATED APPLICATIONS

This application is a continuation-in-part of U.S. application Ser. No. 09/703,474 filed Oct. 31, 2000, which is a continuation-in-part of U.S. application Ser. No. 09/478,124, filed Jan. 5, 2000. The disclosures of each of the foregoing applications are hereby incorporated herein by reference, in their entirety.

### FIELD OF THE INVENTION

The present invention relates to novel targeted delivery systems for bioactive agents, and the use thereof. More particularly, the present invention relates to novel targeted delivery systems for bioactive agents comprising a matrix which comprises a polymer and a targeting ligand.

### BACKGROUND OF THE INVENTION

The formulation and administration of water-insoluble or sparingly water-soluble drugs is generally problematic because of the difficulty, inter alia, of achieving sufficient systemic bioavailability. Low aqueous solubility may result not only in decreased bioavailability, but also in formulations that may lack sufficient stability over extended storage periods. An example in this regard is paclitaxel, available commercially as Taxol® Bristol-Myers Squibb (Princeton, N.J.). Paclitaxel has been shown to exhibit powerful antineoplastic efficacy, particularly for cancers of the breast, ovaries and prostate gland. Due to its limited water solubility, a solvent system has been employed as a delivery system, comprising a mixture of Cremophor EL (polyethoxylated castor oil) and ethanol. However, the use of paclitaxel has been limited in large part due to the side effects of the solvent delivery system. Specifically, the amount of solvent that may be required to deliver an effective dose of paclitaxel is substantial, and Cremophor has been shown to result in serious or fatal hypersensitivity episodes in laboratory animals (see, e.g., Lorenz et al. (1977) Agents Actions 7:63-67) as well as in humans (Weiss et al. (1990) J. Clin. Oncol. 8:1263-1268). Because of the undesirable physiologic reactions associated with paclitaxel-Cremophor formulations, patients are generally premedicated with corticosteroids and/or antihistamines. While premedication has proven to be somewhat effective, mild to moderate hypersensitivity is still a problem in a significant number of patients. (Weiss et al., supra; see also Runowicz et al. (1993) Cancer 71:1591-1596).

Thus, extensive research has been conducted with the aim of producing an improved paclitaxel formulation having reduced toxicity. In particular, efforts have been directed toward (1) modifying the chemistry of the drug itself to make it more hydrophilic and (2) combining the drug with agents that produce water-soluble dispersions. Chemically modified paclitaxel analogs include sulfonated paclitaxel derivatives (see U.S. Pat. No. 5,059,699), amino acid esters (Mathew et al. (1992) J. Med. Chem. 3B:145-151) as well as covalent conjugates of paclitaxel and polyethylene glycol (U.S. Pat. No. 5,648,506 to Desai et al.; Liu et al. (1999) J. Polymer Sci., Part A—Polymer Chem. 37:3492-3503). For the most part, however, research has focused on entrapment of the drug in vesicles or liposomes, and on the incorporation of surfactants into paclitaxel formulations.

Representative liposomal drug delivery systems are described, for example, in U.S. Pat. Nos. 5,395,619, 5,340,588 and 5,154,930. Liposomes, as is well known in the art, are vesicles that may comprise one or more concentrically ordered lipid monolayers or bilayers which encapsulate an aqueous phase. Liposomes form when phospholipids, amphipathic compounds having a polar (hydrophilic) head group covalently bound to a long-chain aliphatic (hydrophobic) tail, are exposed to water. That is, in an aqueous medium, phospholipids generally aggregate to form a structure in which the long-chain aliphatic tails are sequestered within the interior of a shell formed by the polar head groups. Unfortunately, the use of liposomes for delivering many drugs has also proven to be unsatisfactory, in part because liposome compositions are, as a general rule, rapidly cleared from the bloodstream. In addition, even if satisfactory liposomal formulations could be prepared, it may still be necessary to employ a physical release mechanism so that the vesicle may release the active agent in the body before it is taken up by the liver and spleen.

Encasement of paclitaxel microcrystals in shells of biocompatible polymeric materials is described in U.S. Pat. No. 6,096,331 to Desai et al. However, as crystals of hydrophobic drugs may be difficult to dissolve, the rate of drug release in these formulations is generally hard to control.

Incorporation of surfactants into paclitaxel formulations as described, for example, in International Patent Publication No. WO 97/30695, may also be problematic. Surfactants tend to alter the chemistry of a pharmaceutical formulation such that the effective ratio of drug to inactive ingredients is lowered, resulting in the need to increase dosage volume and/or administration time. Additionally, formulations that employ surfactants often readily dissociate upon dilution, e.g., following intravenous injection, resulting in premature drug release. Also, many surfactants are considered unsuitable for parenteral drug administration because of their interaction with cellular membranes.

Also in the prior art, a variety of ligands have been described as useful for targeting specific receptors. Included among these are antibodies (U.S. Pat. No. 5,498,421) and an array of peptides with activity for catalysis of carbohydrate chemistry (WO 00/50477). In order to increase the circulatory lifetime and subsequent bioavailability of these and other ligands, complexation with materials such as polyethylene glycol has proved useful. Most previous derivatization of polyethylene glycol has involved covalent attachment of a drug or biomolecule with or without a spacer moiety. See, e.g., U.S. Pat. No. 5,919,455. Polyethylene glycol has also been used to modify lipids such as dipalmitoylphophatidyl ethanolamine for incorporation into a delivery vehicle such as a liposome. However, as noted above, difficulty has been encountered in preparing suitable delivery systems for such drugs including, for example, liposomal preparations.

Accordingly, there is a need for a new and/or better targeted delivery systems for bioactive agents. The present invention is directed to these, as well as other, important ends.

### SUMMARY OF THE INVENTION

The present invention is directed, in part, to improved targeted delivery systems for bioactive agents. Specifically, in one aspect, there is provided a pharmaceutical composition comprising, in combination with an effective amount of a bioactive agent, a targeted matrix which comprises a polymer and a targeting ligand, wherein the targeting ligand is covalently associated with the polymer and the bioactive agent is associated non-covalently with the polymer, and wherein the bioactive agent is substantially homogeneously dispersed throughout the matrix.

Another aspect of the invention relates to a targeted matrix for use as a delivery vehicle for a bioactive agent, wherein the matrix comprises a polymer that is covalently associated with a targeting ligand.

Yet another aspect of the invention relates to a method for enhancing the bioavailability of a bioactive agent in vivo comprising (i) providing a pharmaceutical composition which comprises, in combination with an effective amount of a bioactive agent, a matrix comprising a polymer and a targeting ligand, and (ii) administering to a patient the pharmaceutical composition, wherein the targeting ligand is associated covalently with the polymer and the bioactive agent is associated non-covalently with the polymer, and wherein the bioactive agent is substantially homogeneously dispersed throughout the matrix.

Still another aspect of the invention relates to a method for treating cancer comprising administering to a patient a pharmaceutical composition comprising, in combination with an effective amount of an anticancer agent, a matrix which comprises a polymer and a targeting ligand, wherein the targeting ligand is covalently associated with the polymer and the anticancer agent is associated non-covalently with the polymer, and wherein the anticancer agent is substantially homogeneously dispersed throughout the matrix.

These and other aspects of the invention will become more apparent from the present specification and claims.

### BRIEF DESCRIPTION OF THE DRAWINGS

For the purpose of illustrating embodiments of the invention, there is shown in the drawings forms which are presently preferred. It should be understood, however, that this invention is not limited to the precise arrangements and instrumentalities shown.

FIG. 1 is a schematic representation of a bioactive agent formulating composition comprising a matrix of a phospholipid conjugated to a linear hydrophilic polymer, namely, dipalmitoylphosphatidylethanolamine (DPPE) linked in to polyethylene glycol 5000 (PEG 5000), in accordance with an embodiment of the present invention. In the figure, “T” represents targeting ligands bound to the free ends of certain of the PEG chains.

FIG. 2 is a schematic representation of a composition, in which a bioactive agent can be formulated, which is a matrix of a highly branched, dendrimeric PEG, in accordance with an alternate embodiment of the present invention. In the figure, “T” represents targeting ligands bound to the free ends of certain of the PEG chains.

FIG. 3 is a schematic representation of a composition, in which a bioactive agent can be formulated, which is a matrix formed from star PEG, in accordance with another alternate embodiment of the present invention. In the figure, “T” represents targeting ligands bound to the free ends of certain of the PEG chains.

FIG. 4 is a schematic representation of a composition, in which a bioactive agent can be formulated, which is a matrix of a lower molecular weight, branched PEG, in accordance with still another alternate embodiment of the present invention. In the figure, “T” represents targeting ligands bound to the free ends of certain of the PEG chains.

FIG. 5 is a branched bioactive agent formulating polymer which contains 8 arms. The branched polymer comprises a block copolymer with an inner more hydrophobic block, e.g. polylactide, and an outer less hydrophobic block, e.g. polyethyleneglycol. In the figure, “T” represents targeting ligands bound to the free ends of certain of the outer PEG arm chains.

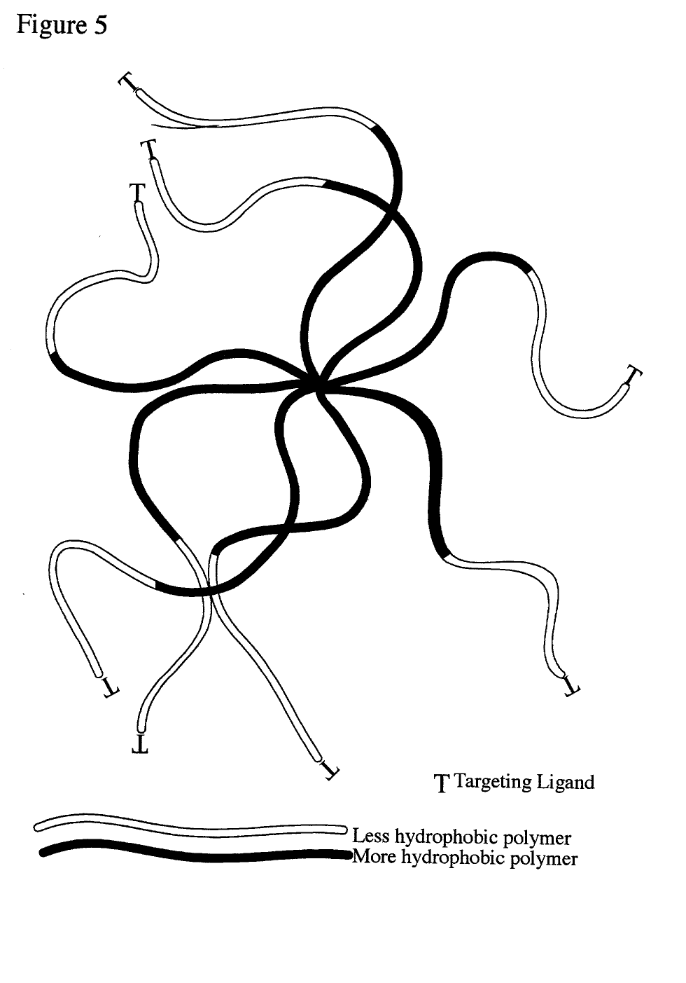

### DETAILED DESCRIPTION OF THE INVENTION

As employed above and throughout the disclosure, the following terms, unless otherwise indicated, shall be understood to have the following meanings. It is also understood that the terminology used herein is for the purpose of describing particular embodiments only and is not intended to be limiting.

“Lipid” refers to a synthetic or naturally-occurring compound which is generally amphipathic and biocompatible. The lipids typically comprise a hydrophilic component and a hydrophobic component. Exemplary lipids include, for example, fatty acids, neutral fats, phosphatides, glycolipids, surface-active agents (surfactants), aliphatic alcohols, waxes, terpenes and steroids.

“Pharmaceutically acceptable” and “biocompatible” refer to compounds, materials, compositions, and/or dosage forms which are, within the scope of sound medical judgment, suitable for contact with the tissues of human beings and animals without causing any undesirable biological effects, including excessive toxicity, irritation, allergic response, or other complications commensurate with a reasonable benefit/risk ratio, and which do not interact in a deleterious manner with any of the other components of the compositions in which it is contained.

“Patient” refers to animals, including mammals, preferably humans.

“Bioactive agent” refers to a substance which may be used in connection with an application that is therapeutic or diagnostic in nature, such as in methods for diagnosing the presence or absence of a disease in a patient and/or in methods for the treatment or prevention of a disease or disorder in a patient. As used herein, “bioactive agent” refers also to substances which are capable of exerting a biological effect in vitro and/or in vivo. The bioactive agents may be neutral or positively or negatively charged. Examples of suitable bioactive agents include diagnostic agents, pharmaceuticals, drugs, synthetic organic molecules, proteins, peptides, vitamins, steroids and genetic material, including nucleosides, nucleotides and polynucleotides.

“Polymer” refers to molecules formed from the chemical union of two or more repeating units. Accordingly, included within the term “polymer” may be, for example, dimers, trimers and oligomers. The polymer may be synthetic, naturally-occurring or semisynthetic. In preferred form, the term “polymer” refers to molecules which comprise 10 or more repeating units. In certain preferred embodiments, the polymers which may be incorporated in the compositions described herein contain no sulfhydryl groups or disulfide linkages.

“Genetic material” refers generally to nucleotides and polynucleotides, including deoxyribonucleic acid (DNA) and ribonucleic acid (RNA). The genetic material may be made by synthetic chemical methodology known to one of ordinary skill in the art, or by the use of recombinant technology, or by a combination of the two. The DNA and RNA may optionally comprise unnatural nucleotides and may be single or double stranded. “Genetic material” refers also to sense and anti-sense DNA and RNA, that is, a nucleotide sequence which is complementary to a specific sequence of nucleotides in DNA and/or RNA.

“Pharmaceutical” or “drug” refers to any therapeutic or prophylactic bioactive agent which may be used in the treatment (including the prevention, diagnosis, alleviation, or cure) of a malady, affliction, disease or injury in a patient. Therapeutically useful peptides, polypeptides and polynucleotides may be included within the meaning of the term pharmaceutical or drug.

“Covalent association” refers to an intermolecular association or bond which involves the sharing of electrons in the bonding orbitals of two atoms.

“Non-covalent association” refers to intermolecular interaction among two or more separate molecules which does not involve a covalent bond. Intermolecular interaction is dependent upon a variety of factors, including, for example, the polarity of the involved molecules, the charge (positive or negative), if any, of the involved molecules, and the like. Non-covalent associations are preferably selected from the group consisting of ionic interaction, dipole-dipole interaction and van der Waal's forces and combinations thereof.

“Ionic interaction” or “electrostatic interaction” refers to intermolecular interaction among two or more molecules, each of which is positively or negatively charged. Thus, for example, “ionic interaction” or “electrostatic interaction” refers to the attraction between a first, positively charged molecule and a second, negatively charged molecule. Exemplary ionic or electrostatic interactions include, for example, the attraction between a negatively charged bioactive agent, for example, genetic material, and a positively charged polymer, for example, a polymer containing a terminal quaternary ammonium salt.

“Dipole-dipole interaction” refers generally to the attraction which can occur among two or more polar molecules. Thus, “dipole-dipole interaction” refers to the attraction of the uncharged, partial positive end of a first polar molecule, commonly designated as δ⁺, to the uncharged, partial negative end of a second polar molecule, commonly designated as δ⁻. Dipole-dipole interactions are exemplified, for example, by the attraction between an electropositive group, for example, a choline head group of phosphatidylcholine, and an electronegative atom, for example, a heteroatom, such as oxygen, nitrogen or sulphur, which is present in the polymer, such as a polyalkylene oxide. “Dipole-dipole interaction” refers also to intermolecular hydrogen bonding in which a hydrogen atom serves as a bridge between electronegative atoms on separate molecules and in which a hydrogen atom is held to a first molecule by a covalent bond and to a second molecule by electrostatic forces.

“Van der Waal's forces” refers to the attractive forces between non-polar molecules that are accounted for by quantum mechanics. Van der Waal's forces are generally associated with momentary dipole moments which are induced by neighboring molecules and which involve changes in electron distribution.

“Hydrogen bond” refers to an attractive force, or bridge, which may occur between a hydrogen atom which is bonded covalently to an electronegative atom, for example, oxygen, sulfur, nitrogen, and the like, and another electronegative atom. The hydrogen bond may occur between a hydrogen atom in a first molecule and an electronegative atom in a second molecule (intermolecular hydrogen bonding). Also, the hydrogen bond may occur between a hydrogen atom and an electronegative atom which are both contained in a single molecule (intramolecular hydrogen bonding).

“Targeting ligand” refers to any material or substance which may promote targeting of tissues and/or receptors in vivo with the compositions of the present invention. The targeting ligand may be synthetic, semi-synthetic, or naturally-occurring. Materials or substances which may serve as targeting ligands include, for example, proteins, including antibodies, glycoproteins and lectins, peptides, polypeptides, saccharides, including mono- and polysaccharides, vitamins, steroids, steroid analogs, hormones, cofactors, bioactive agents, prostacyclin and prostaglandin analogs, and genetic material, including nucleosides, nucleotides and polynucleotides.

“Peptide” or “polypeptide” refer to nitrogenous polymeric compounds which may contain from about 2 to about 100 amino acid residues. In certain preferred embodiments, the peptides which may be incorporated in the compositions described herein contain no sulfhydryl groups or disulfide linkages.

“Protein” refers to a nitrogenous polymer compound which may contain more than about 100 amino acid residues. In certain preferred embodiments, the proteins which may be incorporated in the compositions described herein contain no sulfhydryl groups or disulfide linkages.

“Tissue” refers generally to specialized cells which may perform a particular function. It should be understood that the term “tissue,” as used herein, may refer to an individual cell or a plurality or aggregate of cells, for example, membranes or organs. The term “tissue” also includes reference to an abnormal cell or a plurality of abnormal cells. Exemplary tissues include, for example, myocardial tissue (also referred to as heart tissue or myocardium), including myocardial cells and cardiomyocites, plaques and atheroma, membranous tissues, including endothelium and epithelium, laminae, connective tissue, including interstitial tissue, lung, skin, pancreas, intestine, uterus, adrenal gland and retinal tissues, as well as tumors.

“Angiogenesis” refers to endothelial cells and to proliferation of same as may accompany neoplasia, infection, arthritis, osteoporosis and other inflammatory conditions.

“Intercellular matrix” refers to the region where may be found integrins and other molecules including but not limited to vitronectin, fibronectin, collagen and laminin. These molecules may serve as targets for in accordance with the methods of the present invention, and in certain embodiments may also serve as targeting ligands to other receptors.

“Receptor” refers to a molecular structure within a cell or on the surface of the cell which is generally characterized by the selective binding of a specific substance. Exemplary receptors include, for example, cell-surface receptors for peptide hormones, neurotransmitters, antigens, complement fragments, and immunoglobulins and cytoplasmic receptors for steroid hormones.

“Tumor cells” or “tumor” refers to an aggregate of abnormal cells and/or tissue which may be associated with diseased states that are characterized by uncontrolled cell proliferation. The disease states may involve a variety of cell types, including, for example, endothelial, epithelial and myocardial cells. Included among the disease states are neoplasms, cancer, leukemia and restenosis injuries.

“Alkyl” refers to an aliphatic hydrocarbon group which may be straight, branched or cyclic having 1 to about 10 carbon atoms in the chain, and all combinations and subcombinations of ranges and specific numbers of carbons therein. “Lower alkyl” refers to an alkyl group having 1 to about 4 carbons. The alkyl group may be optionally substituted with one or more alkyl group substituents which may be the same or different, where “alkyl group substituent” includes halo, aryl, hydroxy, alkoxy, aryloxy, alkyloxy, alkylthio, arylthio, aralkyloxy, aralkylthio, carboxy alkoxycarbonyl, oxo and cycloalkyl. There may be optionally inserted along the alkyl group one or more oxygen, sulphur or substituted or unsubstituted nitrogen atoms, wherein the nitrogen substituent is lower alkyl. “Branched” refers to an alkyl group in which a lower alkyl group, such as methyl, ethyl or propyl, is attached to a linear alkyl chain. Exemplary alkyl groups include methyl, ethyl, i-propyl, n-butyl, t-butyl, n-pentyl, heptyl, octyl, decyl, dodecyl, tridecyl, tetradecyl, pentadecyl and hexadecyl. Preferred alkyl groups include the lower alkyl groups of 1 to about 4 carbons. Exemplary cyclic hydrocarbon groups (that is, cycloalkyl groups) include, for example, cyclopentyl, cyclohexyl and cycloheptyl groups. Exemplary cyclic hydrocarbon groups also include cycloalkenyl groups such as, for example, cyclopentenyl and cyclohexenyl, as well as hydrocarbon groups comprising fused cycloalkyl and/or cycloalkenyl groups including for example, steroid groups, such as cholesterol.

“Alkylene” refers to a straight or branched bivalent aliphatic hydrocarbon group having from 1 to about 10 carbon atoms, and all combinations and subcombinations of ranges and specific numbers of carbons therein. “Lower alkylene” refers to an alkylene group having 1 to about 4 carbon atoms. The alkylene group may be straight, branched or cyclic. The alkylene group may be also optionally unsaturated and/or substituted with one or more “alkyl group substituents.” There may be optionally inserted along the alkylene group one or more oxygen, sulphur or substituted or unsubstituted nitrogen atoms, wherein the nitrogen substituent is alkyl as previously described. Exemplary alkylene groups include methylene (—CH₂—), ethylene (—CH₂CH₂—), propylene (—(CH₂)₃—), cyclohexylene (—C₆H₁₀—), —CH═CH—CH═CH—, —CH═CH—CH₂—, and —(CH₂)ₙ-N(R)-(CH₂)ₘ—, wherein each of m and n is independently an integer from 0 to about 10 and R is hydrogen or alkyl.

The present invention is directed, in part, to novel polymeric compositions. Embodiments are provided in which the polymer compositions are in the form of a polymeric matrix, with targeted polymeric matrices, i.e., polymeric matrices that may target tissues, cells and/or receptors in vivo, being particularly preferred. Polymeric matrices within the scope of the present invention may be particularly suitable for use as delivery vehicles for bioactive agents, especially for bioactive agents that may be characterized by limited water solubility. Accordingly, embodiments are provided herein which comprise pharmaceutical compositions which comprise polymeric matrices, preferably targeted polymeric matrices, in combination with a bioactive agent.

The Polymer

The compositions of the present invention comprise, inter alia, a polymer including, for example, hydrophilic polymers and hydrophobic polymers, with hydrophilic polymers being preferred. The term “hydrophilic”, as used herein, refers to a composition, substance or material, for example, a polymer, which may generally readily associate with water. Thus, although the hydrophilic polymers that may be employed in the present invention may have domains of varying type, for example, domains which are more hydrophilic and domains which are more hydrophobic, the overall nature of the hydrophilic polymers is preferably hydrophilic, it being understood, of course, that this hydrophilicity may vary across a continuum from relatively more hydrophilic to relatively less hydrophilic. The term “hydrophobic”, as used herein, refers to a composition, substance or material, for example, a polymer, which generally does not readily associate with water. Thus, although the hydrophobic polymers that may be employed in the present invention may have domains of varying type, for example, domains which are more hydrophobic and domains which are more hydrophilic, the overall nature of the hydrophobic polymers is preferably hydrophobic, it being understood, of course, that this hydrophobicity may vary across a continuum from relatively more hydrophobic to relatively less hydrophobic.

In preferred embodiments, the present polymers may be in the form of a matrix or three-dimensional structure which may be spatially stabilized. The term “matrix”, as used herein, refers to a three dimensional structure which may comprise, for example, a single molecule of a polymer, such as PEG associated with one or more molecules of a bioactive agent, or a complex comprising a plurality of polymer molecules in association with a therapeutic agent. The morphology of the matrix may be, for example, particulate, where the particles are preferably in the form of nanoparticulate structures, or the morphology of the matrix may be micellar. The term “spatially stabilized”, as used herein, means that the relative orientation of a bioactive agent, when present in the matrices of the present invention, may be fixed or substantially fixed in three-dimensional space, without directional specification. Thus, compositions described herein may facilitate physical entrapment and, preferably, immobilization or substantial immobilization, of one or more bioactive agents. Generally, although not necessarily, the spatially stabilized matrix may be sterically constrained. In preferred form, the matrices are hydrophilic, i.e., the overall nature of the matrices is hydrophilic.

Stability may be evaluated, for example, by placing the present pharmaceutical compositions in water, and monitoring for dissolution and/or release of the bioactive agent. Preferably, the present pharmaceutical compositions may be spatially stable for at least about 5 minutes, more preferably at least about 30 minutes, even more preferably for more than an hour. In certain embodiments, the present pharmaceutical compositions may be spatially stable in solution for days, weeks, and even months.

In certain preferred embodiments, the present matrices may comprise a network of particulate structures. The size and shape of the particulate structures may vary depending, for example, on the particular polymer employed, the desired rate of release of the bioactive agent, and the like. For example, the particulate structures may be spherical in shape, or they may take on a variety of regular or irregular shapes. With regard to the size of the particles, in preferred form, the diameter of the particles may range from about 1 nanometer (nm) to less than about 1000 nm, and all combinations and subcombinations of ranges and specific particle sizes therein. More preferably, the diameter of the particles may range from about 10 nm to about 500 nm, with diameters of from about 20 nm to about 200 nm being even more preferred.

A wide variety of polymers may be employed in the present compositions and formulations. Generally speaking, the polymer is one which has the desired hydrophilicity and/or hydrophobicity, and which may form matrices, as well as covalent attachments with targeting ligands, as described in detail herein. The polymer may be crosslinked or non-crosslinked, with substantially non-crosslinked polymers being preferred. The terms “crosslink”, “crosslinked” and “crosslinking”, as used herein, generally refers to the linking of two or more compounds or materials, for example, polymers, by one or more bridges. The bridges, which may be composed of one or more elements, groups or compounds, generally serve to join an atom from a first compound or material molecule to an atom of a second compound or material molecule. The crosslink bridges may involve covalent and/or non-covalent associations. Any of a variety of elements, groups and/or compounds may form the bridges in the crosslinks, and the compounds or materials may be crosslinked naturally or through synthetic means. For example, crosslinking may occur in nature in materials formulated from peptide chains which are joined by disulfide bonds of cystine residues, as in keratins, insulin, and other proteins. Alternatively, crosslinking may be effected by suitable chemical modification, such as, for example, by combining a compound or material, such as a polymer, and a chemical substance that may serve as a crosslinking agent, which are caused to react, for example, by exposure to heat, high-energy radiation, ultrasonic radiation, and the like. Examples include, for example, crosslinking with sulfur which may be present, for example, as sulfhydryl groups in cysteine residues, to provide disulfide linkages, crosslinking with organic peroxides, crosslinking of unsaturated materials by means of high-energy radiation, crosslinking with dimethylol carbamate, and the like. The term “substantially”, as used in reference to crosslinking, means that greater than about 50% of the involved compounds or materials contain crosslinking bridges. In certain embodiments, preferably greater than about 60% of the compounds or materials contain crosslinking bridges, with greater than about 70% being more preferred. Even more preferably, greater than about 80% of the compounds or materials contain crosslinking bridges, with greater than about 90% being still more preferred. In certain particularly preferred embodiments, greater than about 95% of the compounds or materials contain crosslinking bridges. If desired, the substantially crosslinked compounds or materials may be completely crosslinked (i.e., about 100% of the compounds or materials contain crosslinking bridges). In other preferred embodiments, the compounds or materials may be substantially (including completely) non-crosslinked. The term “substantially”, as used in reference to non-crosslinked compounds or materials, means that greater than about 50% of the compounds or materials are devoid of crosslinking bridges. Preferably, greater than about 60% of the compounds or materials are devoid of crosslinking bridges, with greater than about 70% being more preferred. Even more preferably, greater than about 80% of the compounds or materials are devoid of crosslinking bridges, with greater than about 90% being still more preferred. In particularly preferred embodiments, greater than about 95% of the compounds or materials are devoid of crosslinking bridges. If desired, the substantially non-crosslinked compounds or materials may be completely non-crosslinked (i.e., about 100% of the compounds or materials are devoid of crosslinking bridges).

The compositions of the present invention may be advantageously used as delivery vehicles for bioactive agents, particularly bioactive agents that may have reduced or limited solubility in aqueous media. A particular advantage of the present invention is that controlled, sustained release of bioactive agents may be achieved with the compositions described herein. As discussed in greater detail below, the bioactive agent is preferably substantially homogeneously dispersed throughout the present matrices. The term “substantially homogeneously dispersed”, as used herein, means that the bioactive agent may be at least about 75% continuously dispersed throughout the matrix, with about 80% continuous dispersion being preferred. More preferably, the bioactive agent may be at least about 85% continuously dispersed throughout the matrix, with about 90% continuous dispersion being even more preferred. Still more preferably, the bioactive agent may be at least about 95% continuously dispersed throughout the matrix, with about 100% continuous dispersion (i.e., complete dispersion) being especially preferred.

In preferred form, the polymer comprises repeating alkylene units, wherein each alkylene unit optionally contains from one to three heteroatoms selected from —O—, -N(R)- or —S(O)ₙ—, where R is hydrogen or alkyl and n is 0, 1 or 2. Preferably, the alkylene units are ethylene or propylene units. The polymers may be linear (e.g., the type AB, ABA, ABABA or ABCBA, and the like), star (e.g., the type AₙB or BAₙC, and the like, where B is at least n-valent, and n is an integer ranging from about 3 to about 50, and all combinations and subcombinations of ranges and specific integers therein) or branched (e.g., multiple A's depending from one B), with star and branched polymers being preferred. When a branched polymer is employed, particularly when the branched polymer includes an inner, more hydrophobic core region and an outer, more hydrophilic region, the resulting targeted delivery system may be in the form of a soluble complex. An exemplary illustration of such a soluble complex occurs when a branched block copolymer structure binds a plurality of molecules of a bioactive agent, for example, a drug. In this illustration, the structure of the complex does not preferentially comprise a particle but a soluble bioactive agent/copolymer complex which may exhibit micellar characteristics.

The polymers employed in the present matrices may be selected so as to achieve the desired chemical environment to which the bioactive agent may be exposed. Specifically, in the case, for example, of star polymers, the inner core region may generally be relatively more hydrophobic, and the arms or branches may generally be more hydrophilic. It should be understood, however, that the chemical structures of the core, arms and branches of the polymer may be selected, as desired, so as to modify or alter the generally hydrophobic nature of the core (for example, by increasing or decreasing the core's hydrophobicity) and the generally hydrophilic nature of the arms and/or branches (for example, by increasing or decreasing the hydrophilicity of the arms and/or branches).

As noted above, the number of “branches” or “arms” in star polymers may range from about 3 to about 50, with from about 3 to about 30 being preferred, and from about 3 to about 12 branches or arms being more preferred. Even more preferably, the star polymers contain from about 4 to about 8 branches or arms, with either about 4 arms or about 8 arms being still more preferred, and about 8 arms being particularly preferred. Preferred branched polymers may contain from about 3 to about 1000 branches or arms (and all combinations and subcombinations of ranges and specific numbers of branches or arms therein). More preferably, the branched polymers may have from about 4 to about 40 branches or arms, even more preferably from about 4 to about 10 branches or arms, and still more preferably from about 4 to about 8 branches or arms.

In accordance with preferred embodiments, the polymer, whether linear, star or branched, may be selected from the group consisting of a polyalkylene oxide, polyalkyleneimine, polyalkylene amine, polyalkene sulfide, polyalkylene sulfonate, polyalkylene sulfone, poly(alkylenesulfonylalkyleneimine) and copolymers thereof.

As noted above, depending on the particular polymer employed, the polymers may be relatively more hydrophilic or relatively more hydrophobic. Examples of suitable, relatively more hydrophilic polymers include, but are not limited to, polyethylene glycol, polypropylene glycol, branched polyethylene imine, polyvinyl pyrrolidone, polylactide, poly(lactide-co-glycolide), polysorbate, polyethylene oxide, poly(ethylene oxide-co-propylene oxide), poly(oxyethylated) glycerol, poly(oxyethylated) sorbitol, poly(oxyethylated glucose), polymethyloxazoline, polyethyloxazoline, polyhydroxyethyloxazoline, polyhydroxypropyloxazoline, polyvinyl alcohol, poly(hydroxyalkylcarboxylic acid), polyhydroxyethyl acrylic acid, polyhydroxypropyl methacrylic acid, polyhydroxyvalerate, polyhydroxybutyrate, polyoxazolidine, polyaspartamide, polysialic acid, and derivatives, mixtures and copolymers thereof.

Examples of suitable, relatively more hydrophobic polymers include linear polypropylene imine, polyethylene sulfide, polypropylene sulfide, polyethylenesulfonate, polypropylenesulfonate, polyethylene sulfone, polyethylenesulfonylethyleneimine, polycaprolactone, polypropylene oxide, polyvinylmethylether, polyhydroxyethyl acrylate, polyhydroxypropyl methacrylate, polyphosphazene and derivatives, mixtures and copolymers thereof.

Preferred among the foregoing polymers for use in the present compositions are polyethylene glycol (PEG), polypropylene glycol (PPG), and copolymers of PEG and PPG, or PEG and/or PPG containing some fraction of other monomer units (e.g., other alkylene oxide segments such as propylene oxide). Another particularly preferred copolymer is a branched polymer of PEG and PPG, particularly wherein the PPG units comprise the innermost portion of the structure and the PEG units comprise the outer portions of the arms of the branched structure. Also preferred among the foregoing polymers are polysorbates, particularly polysorbate 80 (commercially available as TWEEN® 80), sorbitan mono-9-octadecanoate poly(oxy-1,2-ethanediyl) derivatives.

In a preferred embodiment of the present invention, the branched polymer comprises a block copolymer. The block copolymer may arise from a central core of, for example, a sugar molecule, a polysaccharide or a frame polymer. In preferred form, the block copolymer preferably includes a central core from which radiate about 3 to about 12 arms, with from about 4 to about 8 arms preferred. Preferably, each arm may comprise a block copolymer with an inner, more hydrophobic block and an outer, more hydrophilic block. In preferred embodiments, the inner block may comprise polypropylene oxide, polylactide or polylactide-coglycolide and the outer block comprises polyethylene glycol. Also in preferred embodiments, the targeting ligands may be attached to the outermost portion of the arms.

In an alternate embodiment of the present invention, the polymers employed in the compositions described herein may be polypeptides, i.e., the polymers may comprise repeating units of amino acids. Certain advantages may be achieved in embodiments employing polypeptides in the compositions of the present invention, particularly in embodiments in which hydrophobic domain(s) of the matrices comprise polypeptides. In this connection, peptides may be biodegradable, for example, via the action of enzymes in the body, such as esterases and amidases. Thus, matrices which include polypeptides may exhibit improved metabolism and/or reduced toxicity in the body. In addition, different amino acids or groups of amino acids may be selected, for example, to optimize the interaction of the bioactive agents with the polymeric matrix. For example, amino acids may be selected such that the polypeptide may form a tertiary structure that facilitates wrapping, folding and/or envelopment of the polymer around the bioactive agent. Polyleucine, for example, may form an α-helical structure, that may wrap around a hydrophobic bioactive agent to basically form a tube or tubule around the bioactive agent. The polypeptides employed in the present compositions may be prepared by modern synthetic methods, such as solid phase synthesis and recombinant techniques.

In the case of hydrophobic bioactive agents, polypeptides comprising hydrophobic amino acids may generally be employed, for example, to form a block within the block copolymer, which may preferably comprise both hydrophobic and hydrophilic domains. The polypeptides may be derived from natural, L and D amino acids, as well as unnatural and modified amino acids. In addition, the polypeptides may be fluorinated, i.e., the polypeptides may be substituted with fluorine atoms or fluorinated groups to provide amino acids and polypeptides having a higher degree of hydrophobicity. For example, naturally occurring hydrophobic amino acids, including leucine, isoleucine, valine, proline, alanine, tyrosine and tryptophan, may be used, for example, to provide a homopolymer or a heteropolymer comprising a fragment of hydrophobic amino acids in a polypeptide. The hydrophobic polypeptide may then be covalently attached to a different polymer, for example, a hydrophilic polymer, including the hydrophilic polymers described herein, which in turn may preferably be attached to a targeting ligand, as discussed in detail below.

The length of the polypeptide as well as the particular amino acids employed may be selected, for example, to optimize the interaction between the polypeptide and the bioactive agent including, for example, the extent and the manner in which the polypeptide may envelop, fold or wrap around the bioactive agent. For example, in the case of polyleucine, other amino acids, such as, for example, glycine or proline, may be incorporated into the polypeptide to modify the way the polypeptide bends which may permit increased and more efficient wrapping of the polypeptide around the bioactive agent. Similarly, domains of amino acids may be selected and incorporated in the polypeptide which may improve the chemical interaction or association with the bioactive agent. For example, the drug irinotecan is a lipophilic cation, and the drug camptothecin is hydrophobic although the pyridine residue may be attached to the 10-hydroxy position of camptothecin to provide a pro-drug. The pyridine moiety may also carry a positive charge at physiological pH from the quaternary amine. Incorporating one or more anionic amino acids, for example, glutamate, into the polyleucine polypeptide, may serve to increase the interaction of the predominantly polyleucine polypeptide with camptothecin. In general, for bioactive agents such as irinotecan, which are lipophilic cations, incorporating an anionic segment into the polypeptide may increase the interaction. Conversely, for bioactive agents that are lipophilic anions, one or more cationic amino acids, for example, lysine, arginine or histidine, may be incorporated into the polypeptide. Without intending to be bound by any theory or theories of operation, it is contemplated that the polypeptide may serve as a hydrophobic block which facilitates hydrogen bonding with a bioactive agent containing a charged domain, thereby enabling the formation of a complex, or some other interaction, for example, ion pairing of the polypeptide with the polar, charged portion of the bioactive agent.

While a hydrophobic polypeptide may form a complex or provide other interaction with a given bioactive agent, this is generally insufficient to solubilize the bioactive agent, unless a segment of hydrophilic amino acids is also incorporated into the polypeptide or the polypeptide is otherwise modified, for example, derivatized, to incorporate hydrophilic groups. Solubilization of the hydrophobic bioactive agent/polypeptide matrix may be accomplished, for example, by creating within the polypeptide, not only a block of hydrophobic amino acids, but also a block of hydrophilic or charged amino acids proximate the hydrophobic block. Preferably, however, the hydrophobic segment of amino acids may be covalently bound to another polymer, preferably a hydrophilic polymer, such as polyethyleneglycol (PEG). For example, a decapeptide of polyleucine may be attached to a hydrophilic polymer, such as PEG, for example, via the free amino end of the polyleucine peptide and the free carboxyl end of α-amino, γ-carboxy PEG. The free end of the PEG, via its amino group, may then be used to attach a targeting ligand, for example, a peptide via its terminal carboxyl group. In such embodiments, the hydrophilic polymer, for example, PEG, may vary in length such that it's molecular weight may range, for example, from about 400 to about 100,000 daltons, with molecular weights of from about 1,000 to about 40,000 being preferred. More preferably, the molecular weight of the hydrophilic polymer in the context of the present embodiment, is about 3,500 daltons. Generally speaking, a hydrophilic polymer, such as PEG, having a higher molecular weight, may afford a longer circulation lifetime, but may decrease the affinity of the targeted matrix as the molecular weight increases. Therefore, the molecular weight of the hydrophilic polymer may be is selected for the particular application. It should be noted that, in embodiments involving linear polypeptides, the polymer may be attached to one or both ends of the polypeptide, i.e., to both α-amino and γ-carboxy end groups. Similarly, in the case of attachment of a polymer to both termini of the polypeptide, then the targeting ligand(s) may be attached to one or both termini of the polypeptide-polymer conjugate.

The length of the segment of amino acids in the polypeptide may vary depending, for example, upon the intended application, and the chemistry of the bioactive agent to be delivered, the size of the bioactive agent to be delivered, and the like. In general, at least one hydrophobic amino acid may preferably be incorporated into the polypeptide, but generally the number of amino acids incorporated into the polypeptide may range from about 3 to about 100 amino acids (and all combinations and subcombinations of ranges and specific numbers of amino acids therein). Preferably, the polypeptide comprises from about 5 to about 20 amino acids, with about 10 amino acids being more preferred.

As with the other polymers, including hydrophilic polymers discussed above, the polypeptides may be linear or branched. To create a branched block polypeptide, amino acids with side chains may be used, for example, to first create a backbone. For example, one may start with a backbone of branching amino acids utilizing, for example, the epsilon amino moiety of polylysine or the side chain carboxyl moiety of polyglutamic acid. The backbone may comprise a homopolymer of amino acids or a copolymer of amino acids. Copolymers may be advantageous, for example, in that one or more amino acids can be used as “spacers” to increase the distance between side chains, and thereby minimize steric hindrance or to otherwise optimize properties of the backbone. For example, the backbone may comprise an alternating sequence of lysine with glycine or another amino acid so as to increase the spacing between the side chain bearing amino acids. Preferably, however, when a backbone of branched amino acids is employed, the polymer is in the form of a homopolymer, for example, polylysine or polyglutamate. When a backbone is prepared from the branched amino acids, using peptide chemistry, hydrophobic blocks in the form of pendant peptides may then be attached to the activated side chains of the backbone. In so doing, a branching structure may be created which comprises a plurality of hydrophobic domains. Hydrophilic polymers, such as PEG, may then in turn be attached to the free ends of the pendant chains of hydrophobic amino acids to create a branched block polymer comprised of amino acids and PEG. When such a structure is created from a backbone and multiple chains, then the structure preferably has from about 3 to about 100 arms, more preferably from about 4 to about 20 arms, and still more preferably from about 4 to about 8 arms.

The molecular weight of the polymer employed in the present compositions may vary depending, for example, upon the particular polymer selected, the particular bioactive agent selected, the desired rate of release, and the like. Broadly speaking, the molecular weight of the polymer may range from about 1,000 to about 1,000,000 (and all combinations and subcombinations of ranges and specific molecular weights therein). More preferably, the polymer may have a molecular weight of from about 8,000 to about 100,000, with molecular weights of from about 10,000 to about 40,000 being even more preferred, and a molecular weight of about 20,000 being particularly preferred. Examples of lower molecular weight polymers include polymers such as TWEEN® 80 (about 1,200 daltons) or small branched PEGs on the order of from about 1000 to about 2000 daltons.

With respect to the branched polymers discussed above, the molecular weight of the entire branched polymer may range from about 2000 to about 1,000,000 daltons, preferably from about 5000 to about 100,000 daltons, more preferably from about 10,000 to about 60,000 daltons, and still more preferably about 40,000 daltons. Preferably, each arm has the same unit size of polymer, such as PEG, e.g, about 5000 daltons each for an 8-armed PEG.

In the case of a branched copolymer, the various percentages of the hydrophobic and hydrophilic monomers or blocks in each arm may vary. For example, with an 8 arm branched copolymer of polypropylene glycol (PPG) and PEG, when 50% is PPG and 50% is PEG, both the PPG segment and the PEG segment will have a molecular weight about 2500 daltons, with the PEG forming the outer portion of the arm.

In certain preferred embodiments, the polymer may have a multivalent core structure from which extend arms comprising linear or branched polymers. The cores may preferably be polyhydroxylated monomers such as sugars, sugar alcohols, polyaliphatic alcohols and the like. Preferred among such core structures are neopentanol and polyerythritol, which contain four hydroxy moieties that may be derivatized to afford the various arms or branches. Sugar alcohols such as glycerol, mannitol and sorbitol may also be similarly derivatized.

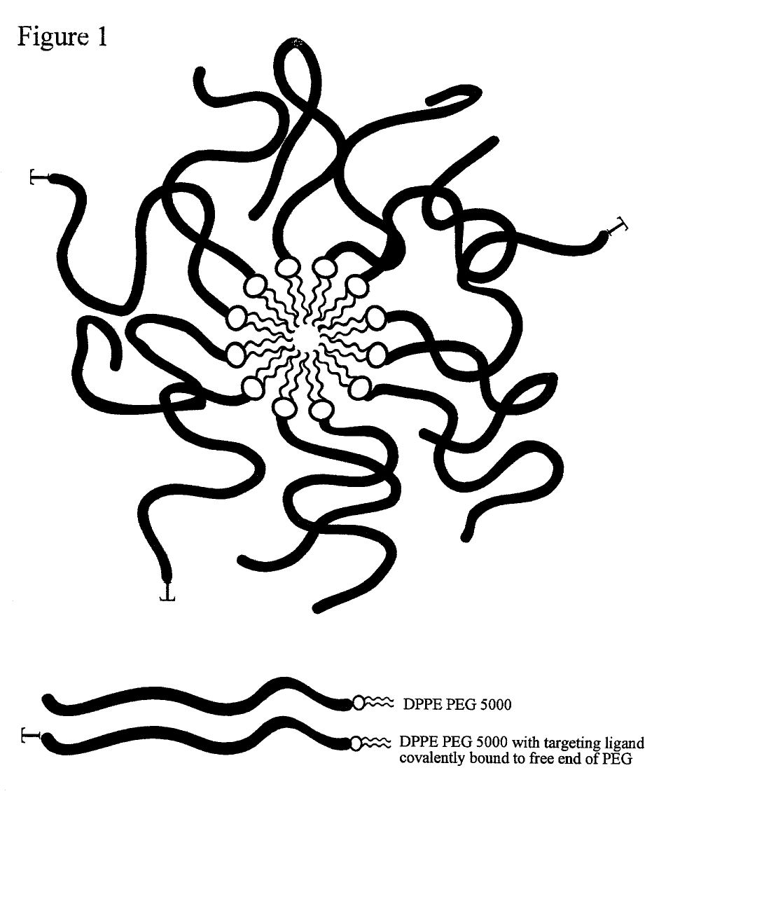

As stated above, a preferred polymer of the present invention is polyethylene glycol which may be either a branched PEG (including “dendrimeric” PEG, i.e., higher molecular weight, highly branched PEG) or star PEG. In certain embodiments, the polymer may be covalently associated with a lipid, such as a phospholipid moiety in which the hydrophobic chains of the phospholipids may tend to associate in an aqueous medium. This is depicted schematically in FIG. 1. Combinations of different types of PEG (e.g., branched PEG and linear PEG, star PEG and linear PEG, branched PEG and phospholipid-conjugated linear PEG, and the like) may also be employed.

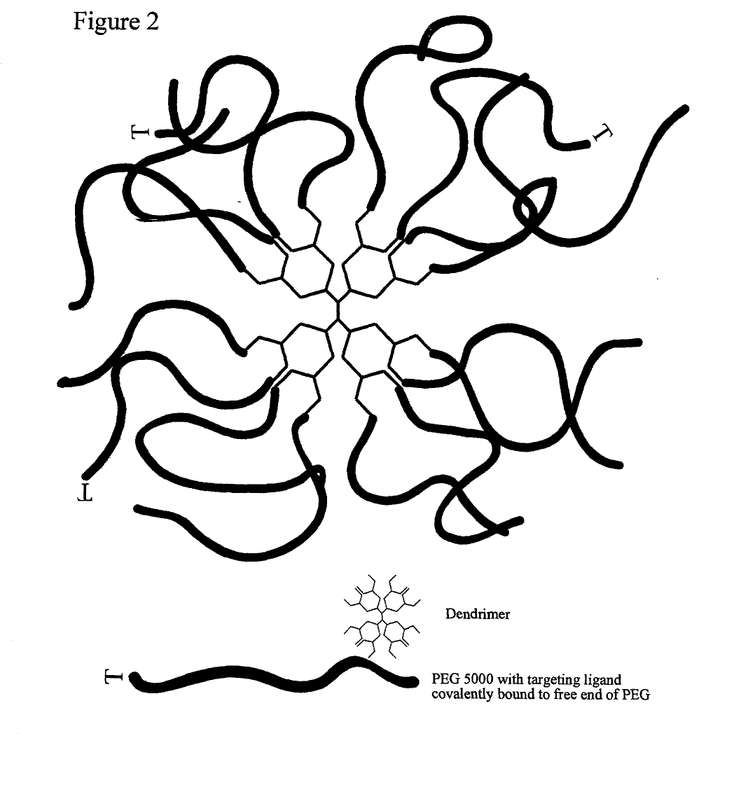

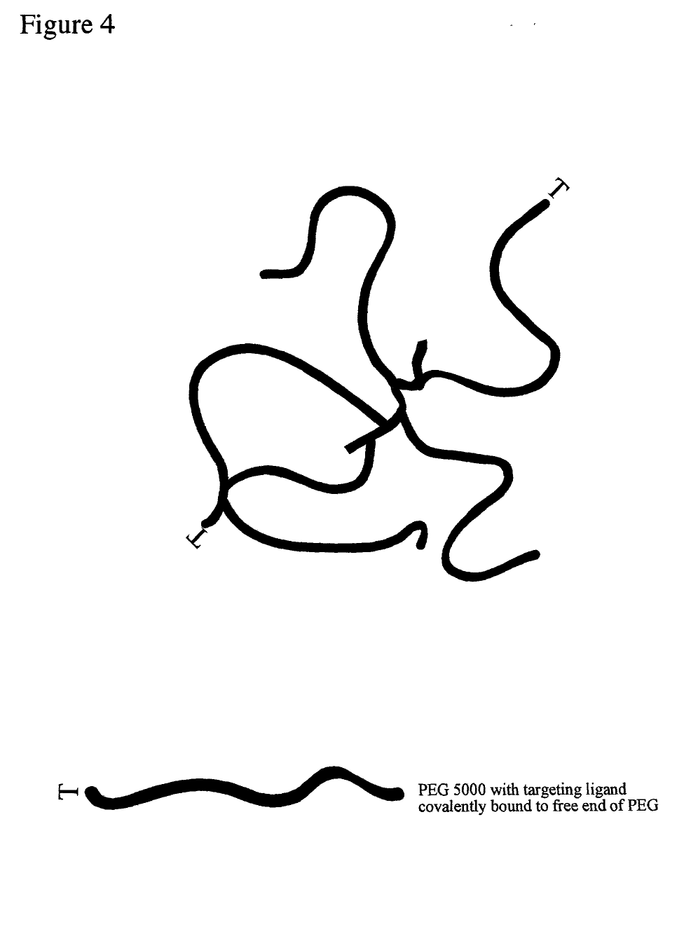

In embodiments involving branched PEG, the branched PEG may have a molecular weight of from about 1000 to about 600,000, preferably from about 2000 to about 100,000, more preferably from about 20,000 to about 40,000. Branched PEG is commercially available, such as from Nippon Oil and Fat (NOF Corporation, Tokyo, Japan) and from Shearwater Polymers (Huntsville, Ala.), or may be readily synthesized by polymerizing lower molecular weight linear PEG molecules (i.e., PEG 2000 or smaller) finctionalized at one or both termini with a reactive group. For example, branched PEG may be synthesized by solution polymerization of lower molecular weight PEG acrylates (i.e., PEG molecules in which a terminal hydroxyl group is replaced by an acrylate functionality, i.e., —O—(CO)—CH═CH₂) in the presence of a free radical polymerization initiator such as 2,2′-azobisisobutyronitrile (AIBN). If desired, mixtures of PEG monoacrylates or monomethacrylates having different molecular weights may be used in order to synthesize a branched polymer having branches or arms of different lengths. Higher molecular weight, highly branched PEG, e.g. branched PEG having a molecular weight of greater than about 10,000 and at least about 1 arm (i.e., one branch point) per 5000 Daltons, may sometimes be referred to herein as dendrimeric PEG. Dendrimeric PEG may preferably be formed by reaction of a hydroxyl-substituted amine, such as triethanolamine, with lower molecular weight PEG that may be linear, branched or star, to form a molecular lattice that may serve as the spatially stabilized matrix for delivery of an entrapped bioactive agent. Dendrimeric structures, including dendrimeric PEG are described, for example, in Liu et al. (1999) PSTT 2(10):393-401, the disclosure of which is hereby incorporated herein by reference, in its entirety. Embodiments involving compositions comprising highly branched, high molecular weight dendrimeric PEG and lower molecular weight branched PEG are schematically illustrated in FIGS. 2 and 4, respectively.

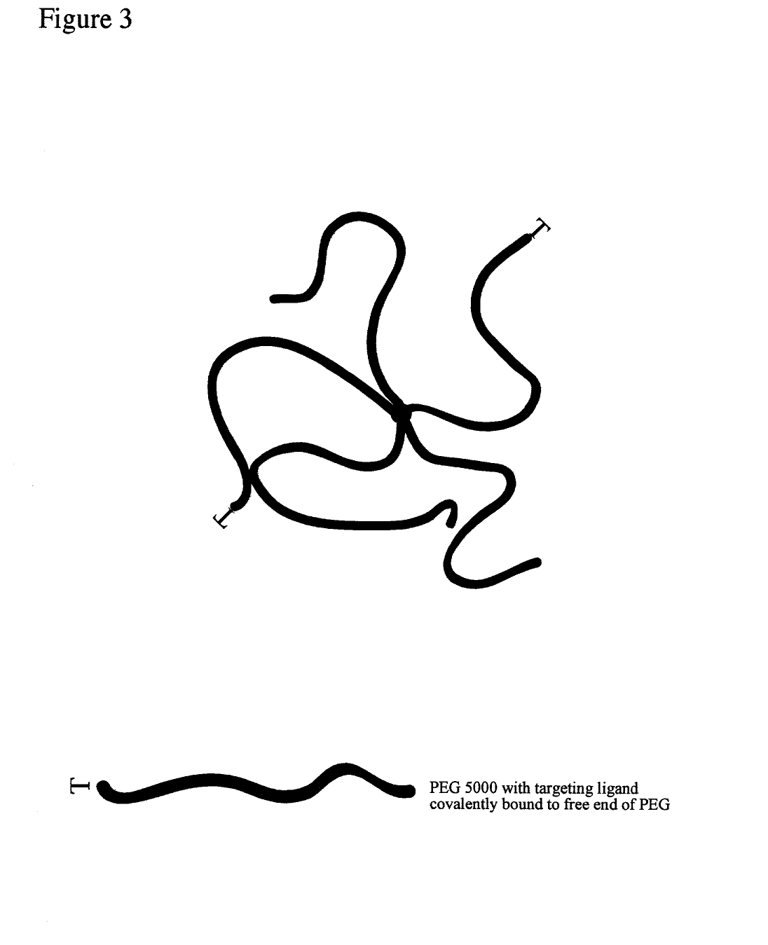

Star molecules of PEG are available commercially (e.g., from Shearwater Polymers, Huntsville, Ala.) or may be readily synthesized using free radical polymerization techniques as described, for example, by Gnanou et al. (1988) Makromol. Chem. 189:2885-2892 and Desai et al., U.S. Pat. No. 5,648,506, the disclosures of which are hereby incorporated herein by reference, in their entireties. Star PEG typically has a central core of divinyl benzene or glycerol. Preferred molecular weights for star molecules of PEG may be from about 1000 to about 500,000 Daltons, with molecular weights of about 10,000 to about 200,000 being preferred. A formulation of the invention which employs star PEG is schematically illustrated in FIG. 3. The bioactive agent may be associated with the branches and/or arms of the matrix, and/or may be associated with the core portions of the matrix structures.

As indicated above, the polymers employed in the present compositions may be linked or conjugated to a lipid, preferably a phospholipid, to provide a polymer-lipid conjugate, as in the case, for example, of PEG-phospholipid conjugates (also referred to as “PEGylated” phospholipids). As with the polymers discussed above, the polymer in the polymer-lipid conjugates, such as polyethylene glycol, may be branched, star or linear. Generally speaking, the molecular weight of the polymer in the polymer-lipid conjugates may be from about 1000 to about 50,000, preferably from about 1000 to about 40,000. It will be appreciated by those skilled in the art that in the case, for example, of polyethylene glycol, the aforementioned molecular weight ranges may correspond to a polymer containing about 20 to about 2000 ethylene oxide units, preferably about 20 to about 1000 ethylene oxide units.

The lipid moiety that may be conjugated to the polymer may be anionic, neutral or cationic, of naturally or synthetic origin, and preferably comprises a phopholipid, preferably a diacyl phosphatidylcholine, a diacyl phosphatidylethanolamine, a diacyl phosphatidylserine, a diacyl phosphatidylinositol, a diacyl phosphatidylglycerol, or a diacyl phosphatidic acid, wherein each acyl moiety can be saturated or unsaturated and will generally be in the range of from about 10 to about 22 carbon atoms in length. Preferred polymer-lipid conjugates are polymer-conjugated diacyl phosphatidyl-ethanolamines having the structure of formula (I):

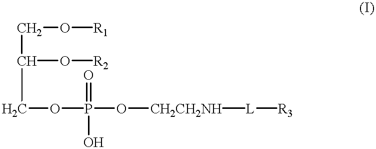

wherein R¹ and R² are the acyl groups, R³ represents the polymer, e.g., a polyalkylene oxide moiety such as poly(ethylene oxide) (i.e., polyethylene glycol), poly(propylene oxide), poly(ethylene oxide-co-propylene oxide) or the like (for linear PEG, R³ is —O—(CH₂CH₂O)ₙ—H), and L is an organic linking moiety such as a carbamate, an ester, or a diketone having the structure of formula (II):

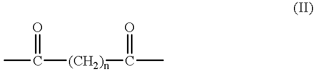

wherein n is 1, 2, 3 or 4. Preferred unsaturated acyl moieties are esters formed from oleic and linoleic acids, and preferred saturated acyl moieties are palmitate, myristate and stearate. Particularly preferred phospholipids for conjugation to linear, branched or star PEG herein are dipalmitoylphosphatidylethanolamine (DPPE) and 1-palmitoyl-2-oleylphosphatidylethanolamine (POPE).

The polymer-lipid conjugates may be synthesized using art-known methods such as those described, for example, in U.S. Pat. No. 4,534,899, the disclosures of which are hereby incorporated herein by reference, in their entirety. For example, preparation of a polymer-lipid conjugate, such as a PEG-phospholipid conjugate, may be carried out by activating the polymer to prepare an activated derivative thereof, having a functional group suitable for reaction with an alcohol, a phosphate group, a carboxylic acid, an amino group or the like. For example, a polyalkylene oxide such as PEG may be activated by the addition of a cyclic polyacid, particularly an anhydride such as succinic or glutaric anhydride (ultimately resulting in the linker of formula (II) wherein n is 2 or 3, respectively). The activated polymer may then be covalently coupled to the selected phosphatidylalkanolamine, such as phosphatidylethanolamine, to give the desired conjugate.

In embodiments in which the polymeric matrix is to be employed as a delivery vehicle for a bioactive agent that may be ionized at physiological pH, charged groups may be inserted into the polymer, for example, to alter or modify the rate at which the bioactive agent may be released from the present compositions. In this connection, the polymer may include charged groups which may have an increased (or decreased) affmity for the bioactive agent. For example, to reduce the rate at which a bioactive agent may be released, and thereby provide sustained delivery over a longer period of time, negatively charged groups, such as phosphates and carboxylates, may be inserted into the polymer for positively charged (e.g., cationic) bioactive agents, while positively charged groups, such as quaternary ammonium groups, may be inserted into the polymer for negatively charged (e.g., anionic) bioactive agents. To insert such groups, a terminal hydroxyl group of a polymer such as PEG may be converted to a carboxylic acid or phosphate moiety by using a mild oxidizing agent such as chromic (VI) acid, nitric acid or potassium permanganate. A preferred oxidizing agent is molecular oxygen used in conjunction with a platinum catalyst. Introduction of phosphate groups may be carried out using a phosphorylating reagent such as phosphorous oxychloride (POCl₃). Terminal quaternary ammonium salts may be synthesized, for example, by reaction with a moiety such as

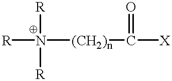

wherein R is H or lower alkyl (e.g., methyl or ethyl), n is typically 1 to 4, and X is an activating group such as Br, Cl, I or an —NHS ester. If desired, such charged polymers may be used to form higher molecular weight aggregates by reaction with a polyvalent counter ion.

Other possible modifications to the polymer include, but are not limited to, the following. A terminal hydroxyl group of a polymer, for example, PEG, may be replaced by a thiol group using conventional means, e.g., by reacting a hydroxyl-containing polymer, such as PEG with a sulfur-containing amino acid such as cysteine, using a protected and activated amino acid. The resulting polymer (“PEG-SH”) is also commercially available, for example from Shearwater Polymers. Alternatively, a mono(lower alkoxy)-substituted polymer, such as monomethoxy polyethylene glycol (MPEG) may be used instead of a non-substituted polymer, e.g., PEG, so that the polymer terminates with a lower alkoxy substituent (such as a methoxy group) rather than with a hydroxyl group. Similarly, an amino substituted polymer, such as PEG amine, may be used in lieu of the corresponding non-substituted polymer, e.g., PEG, so that a terminal amine moiety (—NH₂) may be present rather than a terminal hydroxyl group.

In addition, the polymer may contain two or more types of monomers, as in a copolymer wherein propylene oxide groups (—CH₂CH₂CH₂O—) or polylactide or polylactide-coglycolide have been substituted for some fraction of ethylene oxide groups (—CH₂CH₂O—) in polyethylene glycol. Incorporating propylene oxide, polylactide, polylactide-coglycolide, or polycaprolactone groups may tend to increase the stability of the spatially stabilized matrix, thus decreasing the rate at which the bioactive agent may be released in the body. Generally speaking, increasing the hydrophobicity of the bioactive agent and the fraction of propylene oxide blocks or other hydrophobic blocks such as polylactide or polylactide-coglycolide may result in a slower rate of release of the bioactive agent from the matrix.

The polymer may also contain hydrolyzable linkages to enable hydrolytic degradation within the body and thus facilitate release of the bioactive agent. Suitable hydrolyzable linkages include, for example, any intramolecular bonds that may be cleaved by hydrolysis, typically in the presence of acid or base. Examples of hydrolyzable linkages include, but are not limited to, those disclosed in International Patent Publication No. WO 99/22770, such as carboxylate esters, phosphate esters, acetals, imines, ortho esters and amides. The disclosure of International Patent Publication No. WO 99/22770 is hereby incorporated herein by reference, in its entirety. Other suitable hydrolyzable linkages include, for example, enol ethers, diketene acetals, ketals, anhydrides and cyclic diketenes. Formation of such hydrolyzable linkages within the polymer may be conducted using routine chemistry known to those skilled in the art of organic synthesis and/or described in the pertinent texts and literature. For example, carboxylate linkages may be synthesized by reaction of a carboxylic acid with an alcohol; phosphate ester linkages may be synthesized by reaction of a phosphate group with an alcohol; acetal linkages may be synthesized by reaction of an aldehyde and an alcohol; and the like. Thus a polyethylene glycol matrix containing hydrolyzable linkages “X”

-PEG-X-PEG-

may be synthesized by reaction of -PEG-Y with -PEG-Z wherein Z and Y represent groups located at the terminus of individual PEG molecules and are capable of reacting with each other to form the hydrolyzable linkage X.

Accordingly, it will be appreciated that the rate of release of the bioactive agent from the polymeric matrix may be controlled, for example, by modifying the polymer such as, for example, by adjusting the degree of branching of the polymer, by incorporating different types of monomer units in the polymer structure, by functionalizing the polymer with different terminal groups (which may or may not be charged), and/or by varying the density of hydrolyzable linkages present within the polymeric structure.

In embodiments involving matrices derived, at least in part, from polypeptides, the peptides may be prepared using solid phase or solution chemistry or a combination thereof. For shorter chain polypeptides, such as, for example, less than about 10 or 12 amino acids in length, the peptides may preferably be prepared on a resin using solid phase synthesis techniques. In such embodiments, the peptide, such as, for example, decaleucine, may be prepared and then a hydrophilic polymer, such as PEG, may be coupled to the free end of the homopolymer of amino acids and then, if desired, a targeting ligand may be prepared on the free end of the PEG to thereby create the conjugate polyLeu-PEG-targeting ligand. This conjugate may then be cleaved from the resin and the product isolated, for example, by chromatography. Another block of hydrophilic polymer, for example, PEG, may be coupled to the other terminus of the hydrophobic peptide using solution phase chemistry. Various blocks of the peptides and ligands may be synthesized separately using solid phase chemistry and then stitched together to create larger structures. For example, pentaLeu may be synthesized with solid phase chemistry and four blocks of pentaLeu may then be stitched together to form a 20-mer of polyLeu.

Additionally, specific groups of amino acids may be incorporated into the conjugate to facilitate metabolism by specific enzymes. Enzymes such as the metalloproteinases (e.g. cathepsin-D) are known to hydrolzye specific amino acid sequences. Metalloproteinases, for example, are overexpressed in certain body sites, e.g. in inflammation, angiogenesis and cancer. (Tung, C. H., et al., (1999) Bioconjugate Chem. 10:892-896). Thus, incorporating a cleavable peptide sequence into a conjugate may serve to improve delivery of bioactive agents to the desired tissue. As an example, the octapeptide GPICFRLG or the variant GPIFFRLC is a substrate for cathepsin-D. This peptide may be annealed to the C-terminus of a hydrophobic peptide, such as polyleucine, to generate a site for controlled cleavage. Similarly, endopeptidase sites such as -VLK-, which are sites for plasmin, may be utilized in the construct, for example, to mimic the action of plasmin cleaveage of fibringogen into fibrin during clot formation. Those of skill in the art will readily note that trypsin, chymotrypsin, papain and other endopeptidase-susceptible sites could also be annealed into the construct.

Alternatively, recombinant techniques may be used to prepare polypeptides, including larger chain polypeptides. Yeast or bacteria, for example, may be transfected with a gene encoding the sequence of the polypeptide. This may be particularly advantageous when the polypeptide comprises pure peptidic components. For example, a prototypical polypeptide for use in the present matrices may comprise, for example, a region which binds bioactive agents, and a targeting region. In certain embodiments, the targeting region may serve a two-fold purpose, i.e., not only targeting, but also solubilization of the resulting bioactive agent/matrix. In this regard, complex targeting ligands such as VEG-f may be employed as a bioactive agent-binding region. Recombinant techniques may also be used to produce peptides for isolation and coupling to other materials such as PEG for use in this invention. Variations in the synthetic techniques employed will be apparent to one skilled in the art once armed with the teachings of the present disclosure.

Association of bioactive agents with the polypeptide conjugate may be achieved, for example, according to the particular chemical and physical characteristics of the bioactive agent and the polypeptide conjugate. This may generally be performed, for example, in a solvent in which both the bioactive agent and the polypeptide conjugate are co-miscible. In certain embodiments, this may be an aqueous solution, with appropriate buffers to facilitate interaction, for example, ion pairing between the bioactive agent and the polypeptide. In other embodiments, the solvent employed will be an organic solvent. In still other embodiments, the solvent may be a supercritical fluid such as carbon dioxide. If desired, a mutually immiscible solvent, e.g. water, may be employed, resulting in certain cases in the precipitation of complexes of the bioactive agent and polypeptide. The resulting product may be stored as a lyophilisate, frozen, or as a ready to use aqueous suspension or solution.

The Table below depicts the ability of the amino acids to form turns and tertiary structures and their hydrophobicity. In general, amino acids with preference values greater than about 100 tend to form secondary structures. Amino acids which tend to more hydrophobic, and which may be useful in forming domains for complexing hydrophobic bioactive agents, include amino acids with hydrophobicity values (kcal/mol) of greater than about 0, with hydrophobicity values of greater than about 1 being preferred.

| Amino | | | | | Residue | Residue | Hydrophobicity |
|---|---|---|---|---|---|---|---|
| Acid | ID | P(α) | P(β) | P(turn) | Volume | Area | (kcal/mol) |
| Ala | A | 142 | 83 | 66 | 89 | 115 | 0.42 |
| Arg | R | 98 | 93 | 95 | 173 | 225 | −1.37 |
| Asn | N | 101 | 54 | 146 | 111 | 150 | −0.82 |
| Asp | D | 67 | 89 | 156 | 114 | 160 | −1.05 |
| Cys | C | 70 | 119 | 119 | 109 | 135 | 1.34 |
| Gln | Q | 111 | 110 | 98 | 144 | 180 | −0.3 |
| Glu | E | 151 | 37 | 74 | 138 | 190 | −0.87 |
| Gly | G | 57 | 75 | 156 | 60 | 75 | 0 |
| His | H | 100 | 87 | 95 | 153 | 195 | 0.18 |
| Ile | I | 108 | 160 | 47 | 167 | 175 | 2.46 |
| Leu | L | 121 | 130 | 59 | 167 | 170 | 2.32 |
| Lys | K | 114 | 74 | 101 | 169 | 200 | −1.35 |
| Met | M | 145 | 105 | 60 | 163 | 185 | 1.68 |
| Phe | F | 113 | 138 | 60 | 190 | 210 | 2.44 |
| Pro | P | 57 | 55 | 152 | 113 | 145 | 0.98 |
| Ser | S | 77 | 75 | 143 | 89 | 115 | −0.05 |
| Thr | T | 83 | 119 | 96 | 116 | 140 | 0.35 |
| Trp | W | 108 | 137 | 96 | 228 | 255 | 3.07 |
| Tyr | Y | 69 | 147 | 114 | 194 | 230 | 1.31 |
| Val | V | 106 | 170 | 50 | 140 | 155 | 1.66 |

P(α), P(β), and P(turn) are the Chou-Fasman secondary structure preferences. These preferences were compiled from the distribution of amino acid residues in proteins of known structure. Preferences greater than about 100 are generally considered secondary structure “formers”; the converse is generally true for numbers less than about 100. The residue volumes (Å³) and areas (Å²) are water-accessible values.

From the data above, it is clear that those amino acids with the greater positive hydrophobicity values (i.e., greater than about 1.5) may be preferred for use in the hydrophobic core domains.

Hydrophobicity: These data are ΔΔG values relative to glycine based on the sidechain distribution coefficients (K_{eq}) between 1-octanol and water. Frauchere et al. (1983) Eur. J. Med. Chem. 18, 369-375.

Targeting Ligand

As noted above, the compositions of the present invention further preferably comprise one or more targeting ligands. A wide variety of targeting ligands may be employed in the present compositions depending, for example, on the particular tissue, cell or receptor to be targeted, the particular bioactive agent and/or polymer employed, and the like. Generally speaking, materials which may be employed as targeting ligands include, for example, proteins such as antibodies, peptides, polypeptides, cytokines, growth factors and fragments thereof, vitamins and vitamin analogues such as folate, vitamin-B12, vitamin B6, niacin, nicotinamide, vitamin A and retinoid derivatives, ferritin and vitamin D, sugar molecules and polysaccharides, glycopeptides and glycoproteins, steroids, steroid analogs, hormones, cofactors, bioactive agents, and genetic material, including nucleosides, nucleotides and polynucleotides, drug molecules such as cyclosporin-A, prostaglandin and prostacyclin, and antagonists of the GPIIBIIIA receptor of platelets.

In preferred form, the targeting ligands employed in the present compositions may be covalently associated with the polymer. When multiple targeting ligands are attached to the polymer, the targeting ligands may comprise the same or different ligands. The number of targeting ligands attached to each polymer may vary, depending, for example, on the particular tissue, cells or receptors to be targeted, the targeting ligand and/or polymer selected, and the like. Generally speaking, the number of targeting ligands employed may range from less than about one targeting ligand per polymer molecule to a plurality of targeting ligands per polymer molecule including, for example, up to about several hundred targeting ligands per polymer molecule (and all combinations and subcombinations of ranges and specific numbers of targeting ligands therein). For example, in embodiments in which the matrices comprise nanoparticles, there may be as few as about 1 targeting ligand molecule per every 10 polymer molecules. Generally, the targeting ligands may be covalently attached to any portion of the polymer which may be available to form a covalent bond with a portion of the targeting ligand. For example, the targeting ligands may be covalently attached to the free ends of the polymer molecules, the free ends of the arms of branched polymer molecules, and/or the free ends of arms of star polymer molecules. In the case of branched polymers, the number of targeting ligands attached to the free ends of the branched polymer molecules may vary from less than about one to up to about one hundred targeting ligands per polymer molecule. Preferably, the number of targeting ligands may be about the same as the number of free arms in the branched polymer molecule. For example, in the compositions of the present invention, a branched PEG molecule containing 4 arms may also preferably contain 4 covalently associated targeting ligands, preferably to provide one targeting molecule per arm of PEG. As the branching of the polymer employed increases, the number of targeting ligands associated with the polymer may increase also. Although not preferred, the targeting ligands may also be bound to the backbone portion of the polymer molecules, rather than the free ends.

In preferred embodiments, the targeting ligands employed in the compositions of the present invention may be peptides ranging from about 4 amino acids to about 100 amino acids in length (and all combinations and subcombinations of ranges and specific numbers of amino acids therein). More preferably, the targeting ligands may comprise peptides ranging from about 4 to about 20 amino acids in length, with from about 5 to about 10 amino acids being even more preferred. Still more preferred are peptides containing about 6 or 7 amino acids, i.e., hexapeptides and heptapeptides. The peptides may comprise D and L amino acids and mixtures of D and L amino acids, and may be comprised of all natural amino acids, all synthetic amino acids, and mixtures of natural and synthetic amino acids. The peptides may be synthesized on resins using solid phase synthetic chemistry techniques as are well known in the art, using solution phase chemistry or via recombinant techniques in which organisms such as yeast or bacteria are used to produce the peptide.

Preferred classes of targeting ligands include those which may have specificity for receptors that are associated with cells or tissues, preferably diseased cells or tissue. As used herein, the term “associated with” refers to receptors that are expressed by or present on cells in the tissue. Illustrative of the foregoing types of targeting ligands is the “homing” peptide library, developed from high throughput screening techniques utilizing affinity binding studies. The following exemplary groups of peptides have been shown to exhibit affinity to neural receptors or renal receptors, and may be used to target the present compositions to brain tissue or kidney tissue, respectively:

Brain Homing Peptides: CNSRLHLRC, CENWWGDVC, WRCVLREGPAGGCAWFNRHRL, and CLSSRLDAC.

Kidney Homing Peptides: CLPVASC, and CGAREMC.

Cyclized disulfides of the foregoing brain and kidney homing peptides are particularly preferred.

Peptides recognized by fibronectin- and vitronectin-binding integrins may also be useful as targeting agents in accordance with the present invention. These motifs include the amino acid sequences DGR, NGR, and CRGDC. These peptides are generally characterized by their ability to inhibit integrin-expressing cells from binding to extracellular matrix proteins, and in particular the binding of fibronectin to α5-β1 integrin. Embodiments of these types of peptides include the linear or cyclic peptide motifs CRGDCL, NGR(AHA) and DGR(AHA). The CRGDCL peptide has a high binding affinity, which may make it useful as a general inhibitor and mediator of RGD-dependent cell attachment. Another preferred targeting ligand is the peptide CRGDCA. Both the NGR(AHA) and DGR(AHA) peptides contain the AHA sequence, which is not believed to be essential for binding, as indicated by the parentheses surrounding this sequence. The NGR sequence shows some selectivity toward the α-v-β5 integrin.

Additional peptides which may be useful to bind α5-β1 integrin are those which include the peptide motifs RCDVVV, SLIDIP, and TIRSVD. Peptides which may preferentially bind α5-β1 integrin include the following motifs: KRGD, RRGD, and RGDL.

Peptide sequences which may also be useful as targeting ligands in the present compositions include those which may form -RGD- type binding determinants of antibodies and include the following: CSFGRGDIRNC, CSFGRTDQRIC, CSFGKGDNRIC, CSFGRNDSRNC, CSFGRVDDRNC, CSFGRADRRNC, CSFGRSVDRNC, CSFGKRDMRNC, CSFGRWDARNC, CSFGRQDVRNC, and CSFGRDDGRNC.

To target angiogenic endothelium of solid tumors, suitable targeting ligands include the following peptides: CDCRGDCFC and CNGRCVSGCAGRC.

Other peptide sequences chosen for tissue specificity and which may be useful as targeting ligands in the present invention include the following:

Lung: CGFECVRQCPERC, CGFELETC, CTLRDRNC and CIGEVEVC

Skin: CVALCREACGEGC

Pancreas: SWCEPGWCR

Intestine: YSGKWGW

Uterus: GLSGGRS

Adrenal Gland: LMLPRAD

Retina: CRDVVSVIC and CSCFRDVCC

See, e.g., Rajotte, et. al., (1998) J. Clin. Invest., 102:430-437, the disclosures of which are hereby incorporated herein by reference, in their entirety.

Cationic peptides, including, but not limited to those set out in Table 1 below, are also preferred for use as targeting ligands, particularly due to their specificity for various cancers:

TABLE 1

| GROUP | | | |
|---|---|---|---|
| NAME | PEPTIDE | SEQUENCE | REFERENCE* |
| Abaecins | Abaecin | YVPLPNVPQPGRRPFPTF | Casteels et al. |
| | | PGQGPFNPKIKWPQGY | (1990) |
| Andropins | Andropin | VFIDILDKVENAIHNAAQ | Samakovlis et |
| | | VGIGFAKPFEKLINPK | al.(1991) |
| Apidaecins | Apidaecin 1A | GNNRPVYIPQPRPPHPRI | Casteels et al. |
| | | | (1989) |
| | Apidaecin 1B | GNNRPVYIPQPRPPHPRL | Casteels et al. |
| | | | (1989) |
| | Apidaecin II | GNNRPIYIPQPRPPHPRL | Casteels et al. |
| | | | (1989) |
| AS | AS-48 | 7.4 kDa | Galvez et al. |
| | | | (1989) |
| Bactenecins | Bactenecin | RLCRIVVIRVCR | Romeo et al. |
| | | | (1988) |
| Bac | Bac5 | RFRPPIRRPPIRPPFYPPFR | Frank et al.(1990) |
| | | PPIRPPIFPPIRPPFRPPLRF | |
| | | P | |
| | Bac7 | RRIRPRPPRLPRPRPRPLP | Frank et al.(1990) |
| | | FPRPGPRPIPRPLPFPRPG | |
| | | PRPIPRPLPFFRPGPRPIPR | |
| | | P | |
| Bactericidins | Bactericidin B2 | WNPFKELERAGQRVRDA | Dickinson et al |
| | | VISAAPAVATVGQAALA | (1988) |
| | | RG* | |
| | Bactericidin B3 | WNPFKELERAGQRVRDA | Dickinson et al |
| | | IISAGPAVATVGQAAAIA | (1988) |
| | Bactericidin B4 | WNPFKELERAGQRVRDA | Dickinson et al |
| | | IISAAPAVATVGQAAAIA | (1988) |
| | | RG* | |
| | Bactericidin B-5P | WNPFKELERAGQRVRDA | Dickinson et al. |
| | | VISAAPAVATVGQAAAI | (1988) |
| | | ARGG* | |
| Bacteriocins | Bacteriocin | 4.8 kDa | Takada et al. |
| | C3603 | | (1984) |
| | Bacteriocin | 5 kDa | Nakamura et al. |
| | IY52 | | (1983) |
| Bombinins | Bombinin | GIGALSAKGALKGLAKG | Csordas and Michi |
| | | LAZHFAN* | (1970) |
| | BLP-1 | GIGASILSAGKSALKGLA | Gibson et al. |
| | | KGLAEHFAN* | (1991) |
| | BLP-2 | GIGSAILSAGKSALKGLA | Gibson et al. |
| | | KGLAEHFAN* | (1991) |
| Bombolitins | Bombolitin BI | JKITTMLAKLGKVLAHV* | Argiolas and |
| | | | Pisano (1985) |
| | Bombolitin BII | SKITDILAKLGKVLAIIV* | Argiolas and |
| | | | Pisano(1985) |
| BPTI | Bovine | RPDFCLEPPYTGPCKARII | Creighton and |
| | Pancreatic | RYFYNAKAGLCQTFVYG | Charles(1987) |
| | Trypsin Inhibitor | GCRAKRINNFKSAEDCMR | |
| | (BPTI) | TCGGA | |
| Brevinins | Brevinin-lE | FLPLLAGLAANFLPKIFC | Simmaco et al. |
| | | KITRKC | (1993) |
| | Brevinin-2E | GIMDTLKNLAKTAGKGA | Simmaco et al. |
| | | LQSLLNKASCKLSGQC | (1993) |
| Cecropins | Cecropin A | KWKLFKKIEKVGQNIRD | Gudmundsson et |
| | | GIIKAGPAVAVVGQATQI | al. (1991) |
| | | AK* | |
| | Cecropin B | KWKVFKKIEKMGRNIRN | Xanthopoulas et |
| | | GIVKAGPAIAVLGEAKAL | al. (1988) |
| | | * | |
| | Cecropin C | GWILKKLGKRIERIGQHT | Tryselius et al. |
| | | RDATIQGLGIAQQAANV | (1992) |
| | | AATARG* | |
| | Cecropin D | WNPFKELEKVGQRVRDA | Hultmark et al. |
| | | VISAGPAVATVAQATAL | (1982) |
| | | AK* | |
| | Cecropin P | SWLSKTAKKLENSAKKR | Lee et al. (1989) |
| | | ISEGIAIAIQGGPR | |
| Charybdtoxins | Charybdtoxin | ZFTNVSCTTSKECWSVC | Schweitz et al. |
| | | QRLHNTSRGKCMNKKC | (1989) |
| | | RCYS | |
| Coleoptericins | Coleoptericin | 8.1 kDa | Bulet et al. (1991) |
| Crabolins | Crabolin | FLPLILRKIVTAL* | Argiolas and |
| | | | Pisano (1984) |
| α-Defensins | Cryptbin 1 | LRDLVCYCRSRGCKGRE | Selsted et al. |
| | | RMNGTCRKGHLLYTLCC | (1992) |
| | | R | |
| | Cryptbin 2 | LRDLVCYCRTRGCKRRE | Selsted et al. |
| | | RMNGTCRKGHLMYTLC | (1992) |
| | | CR | |
| | MCP1 | VVCACRRALCLPRERRA | Selsted et al. |
| | | GFCRIRGRIHTPLCCRR | (1983) |
| | MCP2 | VVCACRRALCLPLERRA | Ganz et al. (1989) |
| | | GFCRIRGRIHPLCCRR | |
| | GNCP-1 | RRCICTTRTCRFPYRRLG | Yamashita and |
| | | TCIFQNRVYTFCC | Saito (1989) |
| | GNCP-2 | RRCICTTRTCRFPYRRLG | Yamashita and |
| | | TCLFQNRVYTFCC | Saito (1989) |
| | HNP-1 | ACYCRIPACIAGERRYGT | Lehrer et al. |
| | | CIYQGRLWAFCC | (1991) |
| | HNP-2 | CYCRIPACIAGERRYGTC | Lehrer et al. |
| | | IYQGRLWAFCC | (1991) |
| | NP-1 | VVCACRRALCLPRERRA | Ganz et al. 1989 |
| | | GFCRIRGRIHPLCCRR | |
| | NP-2 | VVCACRRALCLPLERRA | Ganz et al. 1989 |
| | | GFCRIRGRIHPLCCRR | |
| | RatNP-1 | VTCYCRRTRCGFRERLS | Eisenhauer et al. |
| | | GACGYRGRIYRLCCR | (1989) |
| | RatNP-2 | VTCYCRSTRCGFRERLSG | Eisenhauer et al. |
| | | ACGYRGRIYRLCCR | (1989) |
| β-Defensins | BNBD-1 | DFASCHTNGGICLPNRCP | Selsted et al. |
| | | GHMIQIGICFRPRVKCCR | (1993) |
| | | SW | |
| | BNBD-2 | VRNHVTCRINRGFCVPIR | Selsted et al. |
| | | CPGRTRQIGTCFGPRIKC | (1993) |
| | | CRSW | |
| | TAP | NPVSCVRNKGICVPIRCP | Diamond et al. |
| | | GSMKQIGTCVGRAVKCC | (1991) |
| | | RKK | |
| Defensins- | Sapecin | ATCDLLSGTGINHSACAA | Hanzawa et al. |
| insect | | HCLLRGNRGGYCNGKA | (1990) |
| | | VCVCRN | |
| | Insect defensin | GFGCPLDQMQCHRHCQT | Bulet et al. (1992) |
| | | ITGRSGGYCSGPLKLTCT | |
| | | CYR | |
| Defensins- | Scorpion | GFGCPLNQGACHRHCRSI | Cociancich et al. |
| scorpion | defensin | RRRGGYCAGFFKQTCTC | (1993) |
| | | YRN | |
| Dermaseptins | Dermaseptin | ALWKTMLKKLGTMALH | Mor et al. (1991) |
| | | AGKAALGAADTISQTQ | |
| Diptericins | Diptericin | 9 kDa | Reichhardt et al. |
| | | | (1989) |
| Drosocins | Drosocin | GKPRPYSPRPTSHPRPIRV | Bulet et al. (1993) |
| Esculentins | Esculentin | GIFSKLGRKKIKNLLISGL | Simmaco et al. |
| | | KNVGKEVGMDVVRTGJ | (1993) |
| | | DIAGCKIKGEC | |
| Indolicidins | Indolicidn | ILPWKWPWWPWRR* | Selsted et al. |
| | | | (1992) |
| Lactoferricins | Lactoferricin B | FKCRRWQWRMKKLGAP | Bellamy et al. |
| | | SITCVRRAP | (1992b) |
| Lantibiotics | Nisin | ITSISLCTPGCKTGALMG | Hurst (1981) |
| | | CNMKTATCHCSIHVSK | |
| | Pep 5 | TAGPAIRASVKQCQKTL | Keletta et al. |
| | | KATRLFTVSCKGKNGCK | (1989) |
| | Subtilin | MSKFDDFDLDVVKVSKQ | Banerjee and |
| | | DSKITPQWKSESLCTPGC | Hansen |
| | | VTGALQTCFLQTLTCNC | (1988) |
| | | KISK | |
| Leukocons | Leukocin | KYYGNGVHCTKSGCSVN | Hastings et al. |
| | A-val 187 | WGEAFSAGVHRLANGG | (1991) |
| | | NGFW | |
| Magainins | Magainin I | GIGKFLHSAGKFGKAFV | Zasloff (1987) |
| | | GEIMKS* | |
| | Magainin II | GIGKFLHSAKKFGKAFV | Zasloff (1987) |
| | | GEIMNS* | |
| | PGLa | GMASKAGAIAGKIAKVA | Kuchler et al. |
| | | LKAL* | (1989) |
| | PGQ | GVLSNVIGYLKKLGTGA | Moore et al. |
| | | LNAVLKG | (1989) |
| | XPF | GWASKIGQTLGKIAKVG | Sures and Crippa |
| | | LKELIQPK | (1984) |
| Mastoparans | Mastoparan | INLKALAALAKKIL* | Bernheimer and |
| | | | Rudy (1986) |
| Melittins | Melittin | GIGAVLKVLTTGLPALIS | Tosteson and |
| | | WIKRKRQQ | Tosteson (1984) |
| Phormicins | Phormicin A | ATCDLLSGTGINHSACAA | Lambert et al. |
| | | HCLLRGNRGGYCNGKG | (1989) |
| | | VCVCRN | |
| | Phormicin B | ATCDLLSGTGINHSACAA | Lambert et al. |
| | | HCLLRGNRGGYCNRKG | (1989) |
| | | VCVRN | |
| Polyphemusins | Polyphemusin I | RRWCFRVCYRGFCYRKC | Miyata et al. |
| | | R* | (1989) |
| | Polyphemusin II | RRWCFRVCYKGFCYRK | Miyata et al. |
| | | CR* | (1989) |
| Protegrins | Protegrin I | RGGRLCYCRRRFCVCVG | Kokryakov et al. |
| | | R | (1993) |
| | Protegrin II | RGGRLCYCRRRFCICV | Kokryakov et al. |
| | | | (1993) |
| | Protegrin III | RGGGLCYCRRRFCVCVG | Kokryakov et al. |
| | | R | (1993) |
| Royalisins | Royalism | VTCDLLSFKGQVNDSAC | Fujiwara et al. |
| | | AANCLSLGKAGGHCEKG | (1990) |
| | | VCICRKTSFKDLWDKYF | |
| Sarcotoxins | Sarcotoxin 1A | GWLKKIGKKIERVGQHT | Okada and Natori |
| | | RDATIQGLGIAQQAANV | (1985b) |
| | | AATAR* | |
| | Sarcotoxin 1B | GWLKKIGKKIERVGQHT | Okada and Natori |
| | | RDATIQVIGVAQQAANV | (1985b) |
| | | AATAR* | |
| Seminal | Seminalplasmin | SDEKASPDKHHRFSLSRY | Reddy and |
| Plasmins | | AKLANRLANPKLLETFLS | Bhargava (1979) |
| | | KWIGDRGNRSV | |
| Tachyplesins | Tachyplesin I | KWCFRVCYRGICYRRCR | Nakamura et al. |
| | | * | (1988) |
| | Tachyplesin II | RWCFRVCYRGICYRKCR | Muta et al. (1990) |
| | | * | |
| Thionins | Thionin BTH6 | KSCCKDTLARNCYNTCR | Bohimann et al. |
| | | FAGGSRPVCAGACRCKII | (1988) |
| | | SGPKCPSDYPK | |
| Toxins | Toxin 1 | GGKPDLRPCIIPPCHYIPR | Schmidt et al. |
| | | PKPR | (1992) |
| | Toxin 2 | VKDGYIVDDVNCTYFCG | Bontems et al. |
| | | RNAYCNEECTKLKGESG | (1991) |
| | | YCQWASPYGNACYCKLP | |
| | | DHVRTKGPGRCH | |

*Argolas and Pisano, JBC 259:10106 (1984); Argiolas and Pisano, JBC 260:1437 (1985); Banerjec and Hansen, JBC 263:950B (1988); Bellamy et al., J. Appl. Bacter. 73:472 (1992); Bernhelmer and Rudy, BBA 864:123 (1956); Bohimann et al., EMBO J. 7:1559 (1988); Bontems et al., Science 254:1521 (1991);
Bulet ET AL., JBC 266:24520 (1991); Bulet et al., Eur. J. Biochem.209;977 (1992); Bulet et al., JBC 268; 4893 (1993); Casteels et al., EMBO J. 8:2387 (1989); Casteels et al., Eur J. Biochem.187:381 (1990); C Letters 327:231 (1993); Kuchler et al., Eur. J. Biochem. 179:281 (1989); Lambert et al., PNAS 86:262 (1989); Lee et al., PNAS 86:9159 (1989); Lehrer et al.,
Cell 64:229 (1991); Mlyata et al., J. Biochem. 106:663 (1989); Moore et al., JBC 266:1985 (1991); Mor et al., Biochemistry 30:8824 (1991); M Reighhart et al., Eur. J. Biochem. 182:423 (1989); Romeo et al., JRC 263:9573 (1988); Samakovlis et al., EMBO J. 10:163 (1991); Schmidt et al., Schmidt et al., Texican 30:1027 (1992); Schweltz et al., Biochem 28:9708 (1989); Seisied et al., JBC 258:14485 (1983); Selsted et al., JBC 267:4292 (1992); Simmaco et al., FEBS Leit. 324:159 (1993);
Surex and Crippa, PNAS Takada et al., Infact. and imm. 44:370 (1984); Tosteson and Tosteson, Biophysical J. 45:112 (1984); Tryselius et al., Eur. J. Biochem. 204:395 (1992); Xanthopoulos et si., Eur. J. Biochem. 172:371 (1988); Yamashita and Saito, Infect. and Imm. 57:2405 (1989); Zasloff, PNAS 34:5449 (1987). The disclosures of each of the foregoing documents are hereby incorporated herein by refernce, in their entireties.

If desired, the peptides may be cyclized, for example, by (1) sidechain-to-sidechain covalent linkages, including, for example, by the formation of a disulfide linkage via the oxidation of two thiol containing amino acids or analogs thereof, including, for example, cysteine or penicillamine; (2) end-to-sidechain covalent linkages, including, for example, by the use of the amino terminus of the amino acid sequence and a sidechain carboxylate group, such as, for example, a non-critical glutamic acid or aspartic acid group. Alternatively, the end-to-sidechain covalent linkage may involve the carboxylate terminus of the amino acid sequence and a sidechain amino, amidine, guanidine, or other group in the sidechain which contains a nucleophilic nitrogen atom, such sidechain groups including, for example, lysine, arginine, homoarginine, homolysine, or the like; (3) end-to-end covalent linkages that are covalent amide linkages, or the like. Such processes are well known to those skilled in the art. The peptides may also be cyclized via the addition of flanking amino acids. For example, in the case of targeting ligands comprising the tripeptide RGD, flanking amino acids may be added to form (X)n-RGD-(Y)n where n is an integer of from about 1 to about 100 and X and Y may be any natural or synthetic amino acid and, with the proviso that at least one of the involved amino acids is cysteine or an analog such as penicillamine. These targeting ligands may be cyclized via cysteine sidechains with the cyclization occurring through disulfide bonds. Other modes of cyclization may involve end-to-end covalent linkages involving amino to carboxylate peptide bonds. In addition, X may be lysine and/or arginine and Y may be aspartate or glutamate with condensation of the side chain moieties to form a cyclic amide. Additional permutations include side chain group reactions with terminal amino or carboxyl groups.

In addition, “pseudocyclization” may be employed, in which cyclization occurs via non-covalent interactions, such as electrostatic interactions, which induces a folding of the secondary structure to form a type of cyclic moiety. It is contemplated that metal ions may aid the induction of a “pseudocyclic” formation. This type of pseudocyclic formation may be analogous to “zinc fingers.” As known to one of ordinary skill in the art, zinc fingers involve the formation due to electrostatic interactions between a zinc ion (Zn₂₊) and cysteine, penicillamine and/or homocysteine, of a region in the shape of a loop (the finger). In the case of homocysteine, the RGD sequence would reside at the tip of the finger. Of course, it is recognized that, in the context of the present invention, any type of stabilizing cyclization would be suitable as long the recognition and binding peptide ligand, such as, for example, RGD, maintains the proper conformation and/or topography to bind to the appropriate receptor in clots with a reasonable Michaelis-Menten constant (kₘ) or binding constant. As used herein, the term “conformation” refers to the three-dimensional organization of the backbone of the peptide, peptoid, or pseudopeptide, and the term “topography”, as used herein, refers to the three-dimensional organization of the sidechain of the peptide, peptoid, or pseudopeptide.

The targeting ligands may also comprise prostaglandins and prostacyclins, for example, iloprost or prostaglandin D2. For example, the free carboxylic acid group in iloprost may be covalently linked with a polymer, such as PEG, via an ester linkage. Modified PEGs may also react similarly with iloprost to form a thioester, carbamate, amide or ether linkage, depending on the modification of the PEG moiety, as will be appreciated by those of skill in the art, once armed with the teachings of the present disclosure.

In addition to the foregoing exemplary peptide targeting ligands, the targeting ligand may comprise non-peptide, discrete molecules. In preferred form, the discrete molecules comprise compounds which target the vitronectin receptor αvβ3. Discrete molecules which target the vitronectin receptor and which may be suitable for use as targeting ligands in the present methods and compositions include, for example, the following compounds.

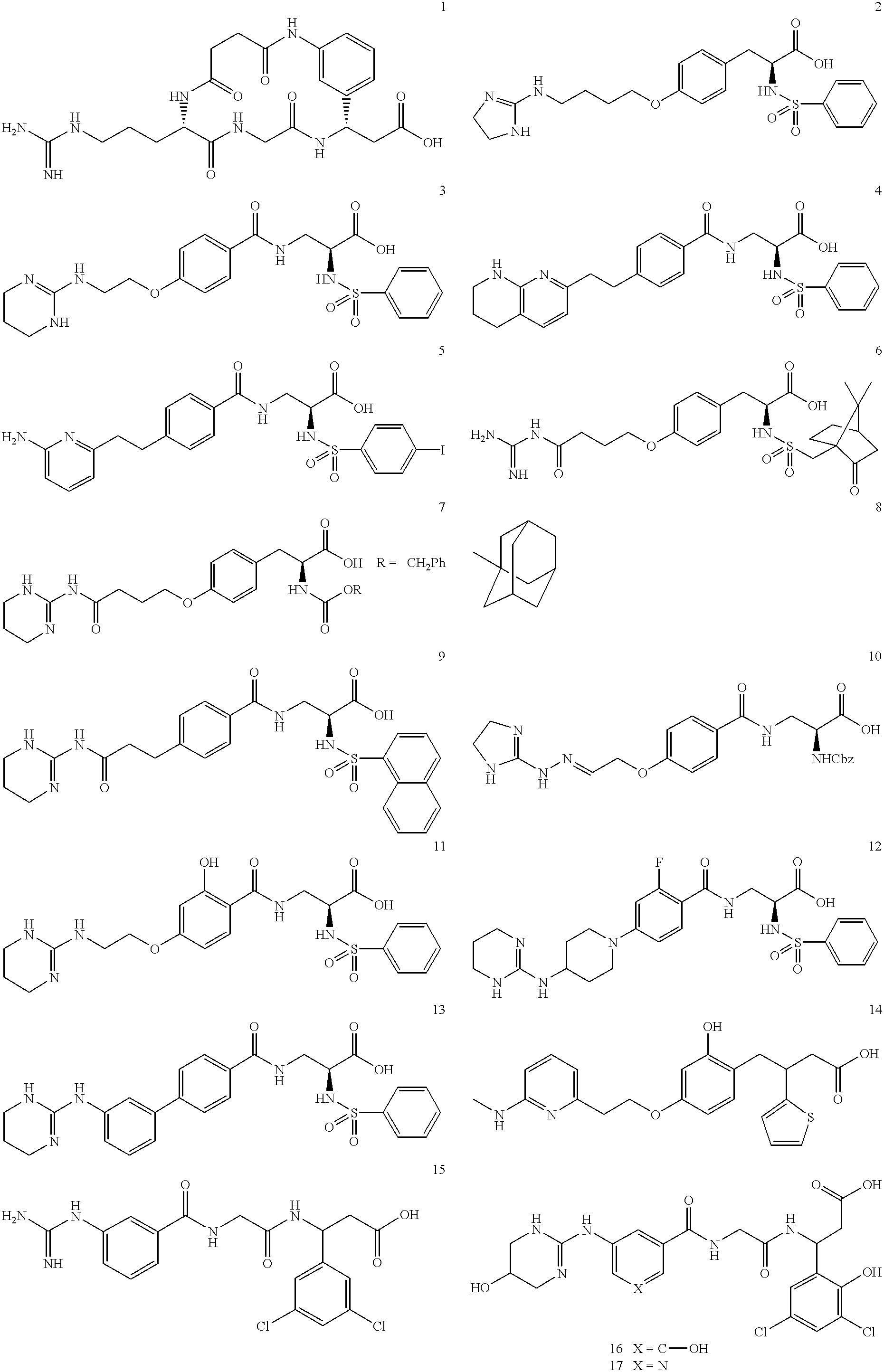

The targeting ligands may be incorporated in the present compositions in a variety of ways which would be apparent to the skilled artisan, once armed with the teachings of the present application. In preferred embodiments, the targeting ligands may be associated with other components of the present compositions, preferably the polymer, covalently. Peptides may be attached to the polymer molecules via their C-terminal or N-terminal groups or via side chains. Solid phase chemistry may be used to attach the peptides to the polymers, for example forming reactions on peptides preformed on a solid matrix, e.g. a resin. Alternatively, solution phase chemistry may be used to attach the peptides to the polymer molecules.

The binding methods used depend on the structure of the targeting moiety. Carbohydrates, hormones and antibodies (or their fragments) are frequently used to direct polymer conjugates to specific cell subsets. Thus, the targeting ligands may preferably include a functional group which may be useful, for example, in forming such covalent bonds. Examples of such functional groups include, for example, amino (—NH₂), hydroxy (—OH), carboxyl (—COOH), thiol (—SH), phosphate, phosphinate, sulfate and sulfinate groups. In the case of cyclized targeting ligands, the ligand preferably includes a functional group, such as amino, hydroxy, carboxyl, thiol, phosphate, phosphinate, sulfate or sulfinate, through which the covalent linkage may be established and which is generally not critical for binding to the desired receptor. Also in the case of cyclized targeting ligands, the cyclization preferably exposes the backbone conformation and sidechain topography of the targeting ligand such as, for example, the sequence RGD, to enable binding of the ligand to the target receptor.

Exemplary covalent bonds by which the targeting ligands may be associated with the polymers include, for example, amide (—CONH—); thioamide (—CSNH—); ether (ROR′, where R and R′ may be the same or different and are other than hydrogen); ester (—COO—); thioester (—COS—); —O—; —S—; —Sₙ—, where n is greater than 1, preferably about 2 to about 8, and more preferably about 2; carbamates; —NH—; -NR-, where R is alkyl, for example, alkyl of from 1 to about 4 carbons; urethane; and substituted imidate; and combinations of two or more of these. Covalent bonds between targeting ligands and polymers may be achieved through the use of molecules that may act, for example, as spacers to increase the conformational and topographical flexibility of the ligand. Examples of such spacers include, for example, succinic acid, 1,6-hexanedioic acid, 1,8-octanedioic acid, and the like, as well as modified amino acids, such as, for example, 6-aminohexanoic acid, 4-aminobutanoic acid, and the like. In addition, in the case of targeting ligands which comprise peptide moieties, sidechain-to-sidechain crosslinking may be complemented with sidechain-to-end crosslinking and/or end-to-end crosslinking. Also, small spacer molecules, such as dimethylsuberimidate, may be used to accomplish similar objectives. The use of agents, including those used in Schiff's base-type reactions, such as gluteraldehyde, may also be employed. The Schiff's base linkages, which may be reversible linkages, can be rendered more permanent covalent linkages via the use of reductive amination procedures. This may involve, for example, chemical reducing agents, such as lithium aluminum hydride reducing agents or their milder analogs, including lithium aluminum diisobutyl hydride (DIBAL), sodium borohydride (NaBH₄) or sodium cyanoborohydride (NaBH₃CN).

The covalent linking of targeting ligands to other components of the present compositions, including the polymers, may be accomplished using synthetic organic techniques which would be readily apparent to one of ordinary skill in the art, based on the present disclosure. For example, the targeting ligands may be linked to the polymers via the use of well known coupling or activation agents. As known to the skilled artisan, activating agents are generally electrophilic. This electrophilicity can be employed to elicit the formation of a covalent bond. Exemplary activating agents which may be used include, for example, carbonyldiimidazole (CDI), dicyclohexylcarbodiimide (DCC), diisopropylcarbodiimide (DIC), methyl sulfonyl chloride, Castro's Reagent, and diphenyl phosphoryl chloride.

The covalent bonds may involve crosslinking and/or polymerization. Crosslinking preferably refers to the attachment of two chains of polymer molecules by bridges, composed of either an element, a group, or a compound, which join certain carbon atoms of the chains by covalent chemical bonds. For example, crosslinking may occur in polypeptides which are joined by the disulfide bonds of the cystine residue. Crosslinking may be achieved, for example, by (1) adding a chemical substance (cross-linking agent) and exposing the mixture to heat, or (2) subjecting a polymer to high energy radiation. A variety of crosslinking agents, or “tethers”, of different lengths and/or functionalities are described, for example, in R. L. Lunbland, Techniques in Protein Modification, CRC Press, Inc., Ann Arbor, Mich., pp. 249-68 (1995), the disclosures of which are hereby incorporated herein by reference, in their entirety. Exemplary crosslinkers include, for example, 3,3′-dithiobis(succinimidylpropionate), dimethyl suberimidate, and its variations thereof, based on hydrocarbon length, and bis-N-maleimido-1,8-octane.

Standard peptide methodology may be used to link the targeting ligand to the polymer when utilizing linker groups having two unique terminal functional groups. As discussed above, bifunctional polymers, and especially bifunctional PEGs, may be synthesized using standard organic synthetic methodologies, and many of these materials are available commercially. More specifically, the polymers employed in the present invention may contain various functional groups, such as, for example, hydroxy, thio and amine groups, which can react with a carboxylic acid or carboxylic acid derivative of the polymeric linker using suitable coupling conditions which would be apparent to one of ordinary skill in the art, once armed with the present disclosure. After the carboxylic acid group (or derivative thereof) reacts with the functional group, for example, hydroxy, thio or amine group to form an ester, thioester or amide group, any protected functional group may be deprotected utilizing procedures which would be well known to those skilled in the art. The term protecting group, as used herein, refers to any moiety which may be used to block reaction of a functional group and which may be removed, as desired, to afford the unprotected functional group. Any of a variety of protecting groups may be employed and these will vary depending, for example, as to whether the group to be protected is an amine, hydroxyl or carboxyl moiety. If the functional group is a hydroxyl group, suitable protecting groups include, for example, certain ethers, esters and carbonates. Such protecting groups are described, for example, in in Greene, TW and Wuts, PGM “Protective Groups in Organic Synthesis” John Wiley, New York, 2nd Edition (1991), the disclosures of which are hereby incorporated herein by reference, in their entirety. Exemplary protecting groups for amine groups include, for example, t-butyloxycarbonyl (Boc), benzyloxycarbonyl(Cbz), o-nitrobenzyloxycarbonyl and and trifluoroacetate (TFA).

Amine groups which may be present, for example, on a polymer may be coupled to amine groups on a peptide by forming a Schiff's base, for example, by using coupling agents, such as glutaraldehyde. An example of this coupling is described by Allcock et al., Macromolecules Vol. 19(6), pp. 1502-1508 (1986), the disclosures of which are hereby incorporated herein by reference, in their entirety. Thus, amino groups in polymers containing same may be activated as described above. The activated amine groups can be used, in turn, to couple to a functionalized polymer, such as, for example, α-amino-ω-hydroxy-PEG in which the ω-hydroxy group has been protected with a carbonate group. After the reaction is completed, the carbonate group can be cleaved, thereby enabling the terminal hydroxy group to be activated for reaction to a suitable targeting ligand. In certain embodiments, a material may be activated, for example, by displacing chlorine atoms in chlorine-containing phosphazene residues, such as polydichlorophosphazine. Subsequent addition of a targeting ligand and quenching of the remaining chloride groups with water or aqueous methanol will yield the coupled product.

In addition, poly(diphenoxyphosphazene) can be synthesized (Allcock et al., Macromolecules Vol. (1986) 19(6), pp. 1502-1508) and immobilized, for example, on DPPE, followed by nitration of the phenoxy moieties by the addition of a mixture of nitric acid and acetic anhydride. The subsequent nitro groups may then be activated, for example, by (1) treatment with cyanogen bromide in 0.1 M phosphate buffer (pH 11), followed by addition of a targeting ligand containing a free amino moiety to generate a coupled urea analog, (2) formation of a diazonium salt using sodium nitrite/HCl, followed by addition of the targeting ligand to form a coupled ligand, and/or (3) the use of a dialdehyde, for example, glutaraldehyde as described above, to form a Schiff's base.

Aldehyde groups on polymers can be coupled with amines as described above by forming a Schiff's base. An example of this coupling procedure is described in Allcock and Austin Macromolecules vol 14. p1616 (1981), the disclosures of which are hereby incorporated herein by reference, in their entirety.

Certain polymers, for example, polysorbates, including TWEEN® polymers, may also be activated for reaction with a targeting ligand by exposure to UV light with free exchange of air, by chemical treatment with anunonium persulfate, or a combination of these methods. Photoactivation may be achieved using a lamp that irradiates at 254 nm or 302 nm, with an output centered at 254 nm being preferred. Longer wave lengths may require longer activation time. While fluorescent room light may also be used for activation, experiments have shown that use of UV light at 254 nm yields maximal activation before room light yields a detectable level of activation.

The atmosphere involved in the photoactivation may also be important. For example, carrying out the activation in an atmosphere of air may double the rate of activation relative to activations performed in an inert atmosphere, or in a sealed environment. A shallow reaction chamber with a large surface area may facilitate oxygen exchange. While it is not yet clear which specific gas is responsible for the increased rates, it is believed that an oxygen derivative is likely. UV exposure of compounds with ether linkages may generate peroxides, which may be detected and quantified using peroxide test strips.

To carry out the photoactivation, the polymer may be placed in a suitable vessel for irradiation. Studies with 2% polysorbate 80 indicate that 254 nm light at about 1800 μW/cm² may be completely absorbed by the solution at a depth of about 3 to about 4 cm. Thus, the activation rate may be maximized by irradiating a relatively thin layer.

As such, a consideration for the vessel is the ability to achieve uniform irradiation. As noted above, a large shallow reaction chamber may be desirable, although this may be difficult to achieve on a large scale. To address this, simple stirring that may facilitate the replenishment of air in the solution may achieve a substantially equivalent result. Thus, if the path length is long or the reaction chamber is not shallow, the reagent may be mixed or agitated. The reagent may be activated in any aqueous solution and buffering may not be required.

An exemplary activation may take place in a cuvette with a 1 cm liquid thickness. The reagent may be irradiated at a distance of less than about 9 cm at about 1500 μW/cm² (initial source output) for about 24 hours.

As noted above, the polyoxyalkylenes may also be activated via chemical oxidation with ammonium persulfate. The activation is typically rapid, and the extent of activation may increase as the concentration of ammonium persulfate is increased. Ammonium persulfate may be used in a range from about 0.01% to about 0.5% (and all combinations and subcombinations of ranges and specific concentrations therein), with from about 0.025 to about 0.1% being preferred. If the levels of ammonium persulfate are too high, the peroxide byproducts may have an adverse effect on the compounds being modified. This adverse effect may be diminished, for example, by treatment of activated polyoxyalkylenes with mercaptoethanol, or another mild reducing agent, which may not inhibit the formation of the product. Peroxides generated from UV treatment may also be reduced by treatment with mercaptoethanol. Furthermore, as noted above, the UV procedure may be performed in conjunction with chemical activation.

The covalent attachment of the polymer to the targeting ligand may be carried out in a liquid or solid phase. Methods that may attach groups via acylation may result in the loss of positive charge via conversion of amino to amido groups.

Some cell receptors recognize both carbohydrates and N-acylated amino-sugars. For example, the asialoglycoprotein receptor on hepatocytes recognizes both galactose and N-acetylgalactosamine. To incorporate galactose into HPMA copolymers, a monomer with protected OH groups, namely 1,2,3,4-O-isopropylidene-6-O-methacryloyl-α-D-galactopyranose may be synthesized, copolymerized with HPMAm and the protecting (isopropylidene) groups may be removed by formic acid. To synthesize polymer conjugates containing N-acylated galactosamine is an easier task. Reactive HPMA coploymer precursors, containing side chains terminated in p-nitrophenyl esters, may be aminolyzed with galactosamine, a reaction which can be performed in DMSO at room temperature.

When using amino groups on the polymeric carrier (or drug) for attachment to aldehyde groups in oxidized saccharide residues of antibodies, oligomers of the latter may be formed by the reaction of amino groups of lysine residues of one antibody molecule with the aldehyde groups of the other. To avoid this side-reaction, hydrazides may be used and the coupling reaction performed at a lower pH where the reactivity of amino groups is minimal.

Larger polypeptides and proteins may also be linked to reactive terminal groups of PEG by methods well-established in the art. Generally, the monomethoxy derivative of PEG is first activated by one of several methods using cyanuric chloride, carbonyl diimidazoles, phenylchloroformate or succinimidyl esters (Mehvar, R., J. Pharm. Pharmac. Sci. (2000) 3:125-136). Included among the proteins or protein fragments that have been derivatized and subsequently reported to retain native activity are monoclonal antibodies or F(ab′)2 fragments, enzymes including arginase, aspariginase, adenosine daminase, uricase, catalase, superoxide dismutase and streptokinase, and growth factors and metabolic potentiators including hG-CSF and recombinant hG-CSF, interleukin 2 and 6, batroxobin, billirubin oxidase, interferon alpha, interferon gamma, trypsin and tissue plasminogen activator.

Those of skill in the art will note that the particular coupling method used to derivatize a particular PEG and a particular protein may depend on the relative sizes of the polymer and protein being used, with the ideal coupling ratio approximating a 1:1 molecular size between the PEG and the protein.

Other methods for covalently linking targeting ligands to other components of the present compositions, including the polymer, in addition to those exemplified above, would be readily apparent to one of ordinary skill in the art, once armed with the teachings of the present disclosure.

Bioactive Agent

As discussed above, the polymeric matrices of the present invention may be advantageously used as a delivery vehicle for one or more bioactive agents. A wide variety of bioactive agents may be included in the compositions of the present invention, including pharmacueticals, such as, for example, anti-neoplastic agents, antibiotics, anti-fungal compounds, cardiac glycosides, immunosuppressive agents, anti-viral agents, steroids, anabolic agents, hormones, anesthetics, neuroleptics, enzyme inhibitors, receptor agonists, antagonists, and/or mixed function agonist/antagonists. Generally speaking, preferred bioactive agents are relatively insoluble in water, and preferably have a greater affinity for the polymer than for aqueous media. For example, preferred bioactive agents include materials that have substantially greater solubility in PEG 400 than in water.

The bioactive agent that may be employed in the present methods and compositions may be any active agent, preferably a bioactive agent whose systemic bioavailability may be enhanced by increasing the solubility of the bioactive agent in water. Generally speaking, the bioactive agent may have a limited water solubility. The term “limited water solubility”, as used herein, means the bioactive agents may be sparingly soluble in aqueous systems, and may exhibit a degree of solubility in systems having increased hydrophobicity, such as polymers, including the polymers described herein. In preferred form, the ratio of the solubility of the bioactive agent in the polymer to the solubility of said bioactive agent in water is greater than about 1:1. More preferably, the ratio of the solubility of the bioactive agent in the polymer to the solubility of said bioactive agent in water is at least about 10:1.

A wide variety of bioactive agents may be incorporated into the compositions of the present invention, and are preferably any compound that has the desired solubility characteristics and which may induce a desired biological effect. Such materials include, for example, the broad classes of compounds normally administered systemically. In general, this includes: analgesic agents; antiarthritic agents; respiratory drugs, including antiasthmatic agents and drugs for preventing reactive airway disease; antibiotics; anticancer agents, including antineoplastic drugs; anticholinergics; anticonvulsants; antidepressants; antidiabetic agents; antidiarrheals; antihelminthics; antihistamines; antihyperlipidemic agents; antihypertensive agents; antiinflammatory agents; antimetabolic agents; antimigraine preparations; antinauseants; antiparkinsonism drugs; antipruritics; antipsychotics; antipyretics; antispasmodics; antiviral agents; anxiolytics; attention deficit disorder (ADD) and attention deficit hyperactivity disorder (ADHD) drugs; cardiovascular preparations including cardioprotective agents; central nervous system stimulants; cough and cold preparations, including decongestants; diuretics; genetic materials; gonadotropin releasing hormone (GnRH) inhibitors; herbal remedies; hormonolytics; hypnotics; immunosuppressive agents; leukotriene inhibitors; mitotic inhibitors; muscle relaxants; parasympatholytics; peptide drugs; psychostimulants; sedatives; steroids; sympathomimetics; tranquilizers; vasodilators, including peripheral vascular dilators; and vitamins.

The methods and compositions of the present invention may also be used to treat bone metabolic disorders. For example, matrices containing the polymers, preferably branched polymers bearing targeting ligands, for example, to αvβIII, may be used to deliver cytostatic and metabolic agents in patients suffering from osteoporosis. Chelating groups may also be incorporated into the polymeric matrix to deliver metal ions for treatment and radiotherapy.

It will be appreciated that the invention may be particularly useful for delivering bioactive agents for which chronic administration may be required, as the present formulations desirably provide for sustained release. The invention is thus advantageous insofar as patient compliance with regard to forgotten or mistimed dosages may be substantially improved. Thus, any biologically active agent that is typically incorporated, for example, into a capsule, tablet, troche, liquid, suspension or emulsion, wherein administration is on a regular (i.e., daily, more than once daily, every other day, or any other regular schedule) can be advantageously delivered using the polymeric matrices of the present invention.

Examples of bioactive agents for which a sustained release formulation is particularly desirable include, but are not limited to, the following:

analgesic agents—hydrocodone, hydromorphone, levorphanol, oxycodone, oxymorphone, codeine, morphine, alfentanil, fentanyl, meperidine and sufentanil, diphenylheptanes such as levomethadyl, methadone and propoxyphene, and anilidopiperidines such as remifentanil;

antiandrogens—bicalutamide, flutamide, hydroxyflutamide, zanoterine and nilutamide;

anxiolytic agents and tranquilizers—diazepam, alprazolam, chlordiazepoxide, clonazepam, halazepam, lorazepam, oxazepam and clorazepate;

antiarthritic agents—hydroxychloroquine, gold-based compounds such as auranofin, aurothioglucose and gold thiomalate, and COX-2 inhibitors such as celecoxib and rofecoxib;

antibiotics (including antineoplastic antibiotics)—vancomycin, bleomycin, pentostatin, mitoxantrone, mitomycin, dactinomycin, plicamycin and amikacin;

anticancer agents, including antineoplastic agents—paclitaxel, docetaxel, camptothecin and its analogues and derivatives (e.g., 9-aminocamptothecin, 9-nitrocamptothecin, 10-hydroxy-camptothecin, irinotecan, topotecan, 20-O-glucopyranosyl camptothecin), taxanes (baccatins, cephalomannine and their derivatives), carboplatin, cisplatin, interferon-2A, interferon-2B, interferon-N3 and other agents of the interferon family, levamisole, altretamine, cladribine, bovine-calmette-guerin (BCG), aldesleukin, tretinoin, procarbazine, dacarbazine, gemcitabine, mitotane, asparaginase, porfimer, mesna, amifostine, mitotic inhibitors including podophyllotoxin derivatives such as teniposide and etoposide and vinca alkaloids such as vinorelbine, vincristine and vinblastine;

antidepressant drugs—selective serotonin reuptake inhibitors such as sertraline, paroxetine, fluoxetine, fluvoxamine, citalopram, venlafaxine and nefazodone; tricyclic anti-depressants such as amitriptyline, doxepin, nortriptyline, imipramine, trimipramine, amoxapine, desipramine, protriptyline, clomipramine, mirtazapine and maprotiline; other anti-depressants such as trazodone, buspirone and bupropion;

antiestrogens—tamoxifen, clomiphene and raloxifene;

antifungals—amphotericin B, imidazoles, triazoles, and griesofulvin;

antihyperlipidemic agents—HMG-CoA reductase inhibitors such as atorastatin, simvastatin, pravastatin, lovastatin and cerivastatin sodium, and other lipid-lowering agents such as clofibrate, fenofibrate, gemfibrozil and tacrine;

antimetabolic agents—methotrexate, fluorouracil, floxuridine, cytarabine, mercaptopurine and fludarabine phosphate;

antimigraine preparations—zolmitriptan, naratriptan, sumatriptan, rizatriptan, methysergide, ergot alkaloids and isometheptene;

antipsychotic agents—chlorpromazine, prochlorperazine, trifluoperazine, promethazine, promazine, thioridazine, mesoridazine, perphenazine, acetophenazine, clozapine, fluphenazine, chlorprothixene, thiothixene, haloperidol, droperidol, molindone, loxapine, risperidone, pimozide and domepezil;

aromatase inhibitors—anastrozole and letrozole;

attention deficit disorder and attention deficit hyperactivity disorder drugs—methylphenidate and pemoline;

cardiovascular preparations—angiotensin converting enzyme (ACE) inhibitors; diuretics; pre- and afterload reducers; iloprost; cardiac glycosides such as digoxin and digitoxin; inotropes such as arninone and milrinone; calcium channel blockers such as verapamil, nifedipine, nicardipene, felodipine, isradipine, nimodipine, bepridil, amlodipine and diltiazem; beta-blockers such as pindolol, propafenone, propranolol, esmolol, sotalol and acebutolol; antiarrhythmics such as moricizine, ibutilide, procainamide, quinidine, disopyramide, lidocaine, phenytoin, tocainide, mexiletine, flecainide, encainide, bretylium and amiodarone; cardioprotective agents such as dexrazoxane and leucovorin;

GnRH inhibitors and other hormonolytics and hormones—leuprolide, goserelin, chlorotrianisene, dinestrol and diethylstilbestrol;

herbal remedies—melatonin;

immunosuppressive agents—6-thioguanine, 6-aza-guanine, azathiopurine, cyclosporin and methotrexate;

lipid-soluble vitamins—tocopherols and retinols;

leukotriene inhibitors—zafirlukast, zileuton and montelukast sodium;

nonsteroidal anti-inflammatory drugs (NSAIDs)—diclofenac, flurbiprofen, ibuprofen, ketoprofen, piroxicam, naproxen, indomethacin, sulindac, tolmetin, meclofenamate, mefenamic acid, etodolac, ketorolac and bromfenac;

peptide drugs—leuprolide, somatostatin, oxytocin, calcitonin and insulin;

peripheral vascular dilator—cyclandelate, isoxsuprine and papaverine;

respiratory drugs—such as theophylline, oxytriphylline, aminophylline and other xanthine derivatives;

steroids—progestogens such as flurogestone acetate, hydroxyprogesterone, hydroxyprogesterone acetate, hydroxyprogesterone caproate, medroxyprogesterone acetate, megestrol, norethindrone, norethindrone acetate, norethisterone, norethynodrel, desogestrel, 3-keto desogestrel, gestadene and levonorgestrel; estrogens such as estradiol and its esters (e.g., estradiol benzoate, valerate, cyprionate, decanoate and acetate), ethynyl estradiol, estriol, estrone, mestranol and polyestradiol phosphate; corticosteroids such as betamethasone, betamethasone acetate, cortisone, hydrocortisone, hydrocortisone acetate, corticosterone, fluocinolone acetonide, flunisolide, fluticasone, prednisolone, prednisone and triamcinolone; androgens and anabolic agents such as aldosterone, androsterone, testosterone and methyl testosterone;

topoimerase inhibitors—camptothecin, anthraquinones, anthracyclines, temiposide, etoposide, topotecan and irinotecan.

immunosuppressive agents such as cycophosphamides as exemplified by cyclosporin-A, mycophenolic acid, rapamycin, 6-mercaptopurine, azothioprine, prednisone, prednisolone, cortisone, azidothymide and OKT-3.

In addition to the foregoing bioactive agents, the present compositions may be useful as delivery vehicles for genetic material, e.g., a nucleic acid, RNA, DNA, recombinant RNA, recombinant DNA, antisense RNA, antisense DNA, hammerhead RNA, a ribozyme, a hammerhead ribozyme, an antigene nucleic acid, a ribo-oligonucleotide, a deoxyribonucleotide, an antisense ribo-oligonucleotide, and an antisense deoxyribo-oligonucleotide. Representative genes include, for example, those which code growth factors and other proteins such as vascular endothelial growth factor, fibroblast growth factor, BCl-2, cystic fibrosis transmembrane regulator, nerve growth factor, human growth factor, erythropoeitin, tumor necrosis factor, and interleukin-2, histocompatibility genes such as HLA-B7, genes coding for enzymes regulating metabolism such as glycolytic enzymes, enzymes of the citric acid cycles and oxidative phosphorylation, genes for hormones such as insulin, glucagon and vasopressin, oncogenes and protooncogenes such as c-fos and c-jun, tumor supression factors such as p53 and telomeres. The genes employed in the compositions may be in the form of gene therapy vectors including, for example, virus-based vectors derived from Adenovirus, adeno-associated virus (AAV), lentiviruses (i.e., retroviruses, such as HIV), herpes simplex virus and, to some extent, vaccinia virus.

The amount of bioactive agent employed in the present compositions may vary and depends, for example, on the particular bioactive agent selected, the polymers employed in the matrix, and the like. Generally speaking, the amount of bioactive agent employed in the present compositions is such that the weight ratio of bioactive agent to all other components of the present compositions is in the range of from about 1:1 to 1:50 (and all combinations and subcombinations of ranges and specific ratios therein). Preferably the weight ratio of bioactive agent to all other components may be from about 1:1 to about 1:20, with a weight ratio of about 1:2.5 to about 1:10 being more preferred, and about 1:5 being particularly preferred.

It may also be desirable to include one or more P-glycoprotein inhibitors in the present compositions. In this connection, it has been shown that P-glycoprotein (P-gp) may be involved in the intestinal absorption of certain drugs including, for example, paclitaxel. Thus, it may be desirable, especially in connection with such bioactive agents, to include in the present compositions a P-gp inhibitor for oral administration, so as to increase its intestinal absorption and thus oral bioavailability. A particularly preferred P-gp inhibitor is cyclosporin A. Other P-gp inhibitors which may be employed in the present compositions would be apparent to one of ordinary skill in the art, once armed with the teachings of the present disclosure.

When employed, the amount of a P-gp inhibitor included in the present compositions may vary depending, for example, on the particular P-gp inhibitor selected, the bioactive agent to be delivered, and the like. Generally speaking, the weight ratio of bioactive agent to P-gp inhibitor may range from about 1:5 to about 5:1 (and all combinations and subcombinations of ranges and specific ratios therein). Preferably the weight ratio of bioactive agent to P-gp inhibitor may be from about 1:2 to about 2:1, with a ratio of about 1:1.5 to about 1.5:1 being more preferred, and a ratio of about 1:1 being particularly preferred. With paclitaxel, it may also be desirable to co-administer a folate (i.e., a salt or ester of folic acid), which may increase paclitaxel absorption.

Manufacture And Storage

The compositions of the present invention may be prepared using any of a variety of suitable methods. Useful methods include, for example, dissolving the bioactive agent and polymer together into a mutually compatible solvent and drying or lyophilizing the material to produce a powder. The resultant powder may be used as is, rehydrated and subjected to a shearing or energy process, e.g. microemulsification or blending. Surpercritical fluids, e.g. carbon dioxide may also be employed as the solvent. The resulting preparation may be spray dried. The polymeric material may also be dissolved or suspended in aqueous media or other solvent and injected in a liquid, e.g. an organic solvent containing the bioactive agent.

Standard techniques and reagents known to those skilled in the art of pharmaceutical formulation and drug delivery may be employed in connection with the preparation of the present compositions. Techniques that may be suitable are described, for example, in Remington: The Science and Practice of Pharmacy, 19^{th} Ed. (Easton, Pa.: Mack Publishing Co., 1995), the disclosure of which is hereby incorporated herein by reference, in their entirety. Remington's discloses, inter alia, conventional methods of preparing pharmaceutical compositions that may be used as described or modified to prepare compositions as described herein. Generally speaking, the polymer, bioactive agent in the case of pharmaceutical compositions, and other optional components, may be combined, for example, by mixing together in an organic solvent or solvent system such as t-butanol, benzene/methanol, ethanol, or an alternative suitable solvent, as will be apparent to those of skill in the art, following by lyophilization of the resulting mixture. The solvent may also be removed by subjecting the mixture to rotary evaporation to yield a powder or a solid matrix. When a solid matrix is obtained, the material may be ground via ball milling or subjected to other mechanical shear stress to achieve a finely ground powder. The resulting powder may be stabilized with surfactants, phospholipids, stabilizing polymers including albumin, and other stabilizing materials. Alternatively, the present compositions may be prepared by spray drying. Spray drying preferably involves the use of a suitable organic solvent, ideally having a flash point sufficiently above the drying temperature. Compositions made using this method are typically in the form of a fluffy, dry powder.

In another preparatory method, the components of the composition may be dissolved in a supercritical fluid, such as compressed carbon dioxide, and then ejected under pressure and shearing force to form the present compositions in the form of dried particles. The resulting composition may be preferably stored in lyophilized form, in which case the lyophilized composition may be rehydrated prior to use. Rehydration may be carried out by mixing the lyophilized composition with an aqueous liquid (e.g., water, isotonic saline solution, phosphate buffer, etc.) to provide a total solute concentration in the range of from about 50 to about 100 mg/ml (and all combinations and subcombinations of ranges and specific solute concentrations therein) and, in the case of pharmaceutical compositions, a bioactive agent concentration in the range of about 1 to about 20 mg/ml (and all combinations and subcombinations of ranges and specific bioactive agent concentrations therein), with a concentration of about 5 to about 15 mg/ml being preferred. The compostions may, however, be stored in the aqueous state, e.g., in pre-filled syringes or vials, and may also be stored in a physiologically acceptable organic solvent such as ethanol, propylene glycol or glycerol, to be diluted with aqueous media prior to administration to a patient. The lyophilized and rehydrated formulations may be stored at various temperatures such as freezing conditions (below about 0° C. and as low as about −40° to about −100° C.), refrigerated conditions generally from about 0° C. to about 15° C., room temperature conditions generally from about 15° C. to about and 28° C., or at elevated temperatures as high as about 40° C.

The particle size of individual particles within the formulation will vary and may depend, for example, upon the molecular weight and concentration of the selected polymer, the concentration of bioactive agent, as well as its solubility profile (i.e., its solubility in water and the polymer), the use of additional stabilizing polymers, such as albumin, and the conditions used in manufacturing. For example, stabilizing polymers and various excipients well known to those skilled in the art may be used to facilitate rehydration and provide a substantially homogeneous dispersion. Additionally, mechanical processing techniques can be used to adjust particle size to the appropriate diameter for the intended application; for example, after rehydration, the compositions may be subjected to shear forces with microfluidization, sonication, extrusion, or the like.

As noted above, the diameter of the nanoparticles may range from about 1 nm to less than about 1000 nm, and all combinations and subcombinations of ranges and specific particle sizes therein. With regard to compositions employed using a stabilizing polymer, the particulates may be sized on the order of about 20 nm to about 100 nm. These smaller particles, by virtue of their larger accessible surface-to-volume ratio, tend to release bioactive agent quite rapidly, while larger particles, e.g., for example, particles greater than about 10 μm in diameter, may provide for a more gradual, sustained release of bioactive agent. For intramuscular and subcutaneous injection, a preferred particle size may range from about 1 nm to about 500 μm, more preferably from about 10 nm to about 300 μm, and even more preferably from about 20 μm to about 200 μm. For intravenous administration, a preferred particle size may range from about 30 nm to about 250 nm. For interstitial administration and fracture or wound packing, preferred particle sizes may be up to about 1000 μm, while for embolization, particle sizes may generally range from about 100 μm to about 250 μm.

The compositions may can be sterilized using either heat, ionizing radiation or filtration. For bioactive agents that are thermally stable, heat sterilization may be preferable. Lower viscosity compositions may be filter sterilized, in which case the particle size may preferably be under about 200 nm. Aseptic manufacturing conditions may be employed as well, and lyophilization is also helpful to maintain sterility and ensure long shelf-life. In addition, anti-bacterial agents may be included in aqueous compositions to prevent or reduce bacterial contamination.

Utility

The pharmaceutical compositions of the present invention may be administered by any means that results in the contact of the bioactive agent with the agent's site or site(s) of action in the body of a patient. The compositions may be administered by any conventional means available for use in conjunction with pharmaceuticals, either as individual therapeutic agents or in a combination of therapeutic agents. For example, the present pharmaceutical compositions may be administered alone, or they may be used in combination with other therapeutically active ingredients.

The compounds are preferably combined with a pharmaceutical carrier selected on the basis of the chosen route of administration and standard pharmaceutical practice as described, for example, in Remington's Pharmaceutical Sciences (Mack Pub. Co., Easton, Pa., 1980), the disclosures of which are hereby incorporated herein by reference, in their entirety.

Pharmaceutical compositions of the present invention can be administered to a mammalian host in a variety of forms adapted to the chosen route of administration, e.g., orally or parenterally. Parenteral administration in this respect includes administration by the following routes: intravenous, intramuscular, subcutaneous, intraocular, intrasynovial, transepithelial including transdermal, ophthalmic, sublingual and buccal; topically including ophthalmic, dermal, ocular, rectal and nasal inhalation via insufflation, aerosol and rectal systemic.

The pharmaceutical compositions may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the compositions may be used in the form of ingestible tablets, buccal tablets, troches, capsules, elixirs, suspensions, syrups, wafers, and the like. The amount of bioactive agent(s) in such therapeutically useful compositions is preferably such that a suitable dosage will be obtained. Preferred compositions according to the present invention may be prepared so that an oral dosage unit form contains from about 0.1 to about 1000 mg of bioactive agent.

The tablets, troches, pills, capsules and the like may also contain one or more of the following: a binder, such as gum tragacanth, acacia, corn starch or gelatin; an excipient, such as dicalcium phosphate; a disintegrating agent, such as corn starch, potato starch, alginic acid and the like; a lubricant, such as magnesium stearate; a sweetening agent such as sucrose, lactose or saccharin; or a flavoring agent, such as peppermint, oil of wintergreen or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavoring, such as cherry or orange flavor. Of course, any material used in preparing any dosage unit form is preferably pharmaceutically pure and substantially non-toxic in the amounts employed. In addition, the active compound may be incorporated into sustained-release preparations and formulations.

The pharmaceutical compositions may also be administered parenterally or intraperitoneally. Suitable compositions may be prepared in water suitably mixed with a surfactant, such as hydroxypropylcellulose. A dispersion can also be prepared in glycerol, liquid polyethylene glycols and mixtures thereof and in oils. Under ordinary conditions of storage and use, these preparations may contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. In all cases, the form is preferably sterile and fluid to provide easy syringability. It is preferably stable under the conditions of manufacture and storage and is preferably preserved against the contaminating action of microorganisms such as bacteria and fungi. The carrier may be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating, such as lecithin, by the maintenance of the required particle size in the case of a dispersion, and by the use of surfactants. The prevention of the action of microorganisms may be achieved by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions may be achieved by the use of agents delaying absorption, for example, aluminum monostearate and gelatin.

Sterile injectable solutions may be prepared by incorporating the pharmaceutical compositions in the required amounts, in the appropriate solvent, with various of the other ingredients enumerated above, as required, followed by filtered sterilization. Generally, dispersions may be prepared by incorporating the compositions into a sterile vehicle which contains the basic dispersion medium and the required other ingredients from those enumerated above. In the case of sterile powders for the preparation of sterile injectable solutions, the preferred methods of preparation may include vacuum drying and the freeze drying technique which yield a powder of the active ingredient, plus any additional desired ingredient from the previously sterile-filtered solution thereof.

The dosage of the pharmaceutical compositions of the present invention that will be most suitable for prophylaxis or treatment will vary with the form of administration, the particular bioactive agent chosen and the physiological characteristics of the particular patient under treatment. Generally, small dosages may be used initially and, if necessary, increased by small increments until the desired effect under the circumstances is reached. Generally speaking, oral administration may require higher dosages.

The present compositions may also be useful as packing materials for wounds and fractures, and as coating materials for endoprostheses such as stents, grafts and joint prostheses. For example, the present compositions may be employed as coating materials for endoprostheses to provide local delivery of a bioactive agent to provide local delivery following coronary intervention.

### EXAMPLES

The invention is further demonstrated in the following examples. Examples 1, 2, 3, 12, 13 and 18 are actual examples and Examples 4 to 11, 14 to 17, 19 20 and 21 are prophetic examples. The examples are for purposes of illustration and are not intended to limit the scope of the present invention.

Example 1

This example is directed to the preparation of the peptide CRGDC.

A. Preparation of Cyst(trt)-Wang resin.

Into a 250 mL round bottom flask were added 3.33 g of Fmoc-Cyst(trt)-OH (5.7 mmoles, 2.0 equiv.) (Advanced Chemtech, Louisville, Ky.) and 872 mg (5.7 mmoles, 2.0 equiv) of hydroxybenzotriazole (Chem-hnpex) dissolved in a minimal amount of dimethyformamide (DMF). Into a separate vessel were added 719 mg (5.7 mmoles, 2 equiv.) of diisopropylcarbodiimide (DIC) and approx. 50 mg (0.28 mmoles, 0.1 equiv.) dimethylaminopyridine (DMAP) (Aldrich, Milwaukee, Wis.) This was dissolved in a 10:1 v:v mixture of methylene chloride (DCM) : DMF. Finally, 3.0 g (2.85 mmoles, 1 equiv.) of Wang resin (Advanced Chemtech, Louisville, Ky.) were added to the mixture of DIC, DMAP and DMF. The two vessels were then combined and heated on an oil bath to approximately 55° C. and allowed to mildly reflux for 24 hours with occasionally swirling (no stirring). The resin was then separated by filtration and consecutive washings (3×) with DMF, DCM, MeOH, and finally DCM again. The resin was dried to yield 5.17 g of product with calculated substitution of 0.72 mmoles G-1. The resin was then reacted with 0.5 mL of acetic anhydride and 0.5 mL of triethylamine in DCM to cap remaining free hydroxyl groups.

The resulting Fmoc-Cys (trt)-Wang resin was deprotected using Fmoc strategy by addition in the following order: (1) deprotect with 23% v:v diisopropylethylamine (DIEA) in N-methylpyrrolidinone (NMP); (2) wash with DCM (3×), MeOH (3×), DCM (3×); and (3) addition of 3 equivalents of DIC, HOBT, and Fmoc-Asp (tBu)-OH. The resin was then reacted for approximately 3 to 24 hours and monitored for completeness using the method of Kaiser.

The resin-bound peptide was cleaved from the resin by stirring in an ice-cold solution of 0.82 mL trifluoroacetic acid (TFA), 0.25 mL ethanedithiol, 0.25 mL water, and 0.5 g phenol for every 1.0 g of resin. The resin was stirred for 90 minutes. The filtrate was separated and was then added dropwise to an ice-cold solution of ether (Mallinckrodt, St. Louis, Mo.). The white precipitate was then filtered from the ether phase and dried in vacuo. The white powder was then diluted with distilled-deionized water followed by adjustment of the pH to approx. 8.0. To this was added in a dropwise fashion, 0.01 M potassium ferricyanide (K₃FeCN₆). Addition was continued with intermittent adjustment of the pH to appxox. 8.0. Addition was discontinued when the yellow color, indicative of K₃FeCN₆, no longer disappeared. The resulting cyclized peptide was then stirred with Amberlite AG-78 (Aldrich) until the yellow tint was no longer visible. The exchange resin was then filtered off through a coarse scintered-glass finnel followed by concentration of the product in vacuo.

The peptide was then purified by HPLC using a linear greadient of 0.1% TFA followed by enrichment with acetonitrile. The purified peptide was isolated and dried by lyophilization to yield cyclic CRGDC in good yield.

Example 2

This example is directed to the preparation of phosphorylated PEG.

Branched PEG (4-arms, 20 kD, Shearwater Polymers, Huntsville, Ala.) (0.529 g) was dissolved in 10 mL acetonitrile (EM Science, HPLC grade) in a 25 mL round bottomed flask. Twenty microliters of triethylamine (Sigma Chemical; 1.43×10⁻⁴ mol) was added into the PEG/acetonitrile solution. Five microliters of phosphorous oxychloride (POCl₃) (Aldrich Chemical) was then added to 7 mL of acetonitrile in a side arm addition funnel and slowly allowed to drip into the stirred PEG/acetonitrile solution over 15 minutes. After 12-14 hrs of stirring at ambient temperature, the reaction mixture was quenched with 25 mL H₂O. The contents were then dialyzed against H₂O for 12 hours with 2 changes of dialysis bath. The dialysate was then quick frozen and lyophilized. Elemental microanalysis for C, H, and P in the resulting white flaky powder indicated that one or two ends of the branches were phosphorylated. The phophorylated PEG 2000 was reacted with 1.5 equivalent of carbonyldiimidazole to from the mixed anhydride in the DCM. The precipitated carbonylimidazole was removed by filtration.

This example was repeated using twice the amount of POCl₃. In the subsequent analysis, approximately 30% of the PEG showed phosphorylation on all four arms. The resulting compound was separated from the incompletely phosphorylated PEG adducts via ion-exchange chromatography.

Example 3

This example is directed to the preparation of FMOC-PPG-NHS.

Step 1: Polypropyleneglycol (PPG), MW 3500 (Aldrich Chemical) was reacted with 1 equivalent of FMOC Glycine (American Peptide Company, Inc., Sunnyvale, Calif.), 1 equivalent of DIC and HOBT in DCM at room temperature for 4 hours. The product, HO-PPG-Glycine-FMOC, was purified by standard chromatographic techniques.

Step 2: The product from Step 1 was reacted with 1 equivalent of PBr₃ (Aldrich Chemical) in THF with a trace of HCl at RT for 8 hours. The product, Br-PPG-Glycine-FMOC, was isolated and purified.

Step 3: Br-PPG-Glycine-FMOC from Step 2 was next reacted with one equivalent of chloroacetic acid and 2 equivalents of sodium hydroxide for 90-120 minutes at room temperature. The reaction was quenched by addition of sodium dihydrigephosphate and adjusting the pH to 7.0. The product was then purified by dialysis.

Step 4: The end carboxylate was activated by reacting the unprotected end group (carboxlate group) with 1 equivalent of N-hydroxysuccinimide in the presence of DIC in DCM for 4 hours. The product was then purified by dialysis.

Example 4

This example is directed to the preparation of CRGDC-branched PEG.

The preparation of CRGDC described in Example 1 is repeated followed by deprotection of the terminal Fmoc on the cysteine. After washing with DCM, MeOH, and DCM, the resin is then treated with three equivalents of DIC and one equivalent of phosphorylated branched PEG 2000 mixed anhydride from Example 2. The resin is reacted for four hours and coupling is tested for completion using the method of Kaiser.

The resulting product is cleaved from the resin using the same TFA, EDT, phenol, water cocktail as described in Example 1, followed by dilution of the solution and adjustment of the pH to 8.0. The peptide portion is then cyclized using the potassium ferricyanide cyclization procedure described in Example 1. The aqueous mixture is then dialyzed through a 1000 MWCO membrane bag followed by concentration in vacuo. The product is purified by HPLC using a C-18 reverse phase HPLC column (Vydac TP-1010 C-18 preparatory column) and a water-methanol eluting system, and isolated by fraction collection and concentration in vacuo.

Example 5

This example is directed to the preparation of CRGDC-Branched PEG-amine.

Branched PEG (4 Arm, 20 K, Shearwater Corporation) is reacted with 4 equivalents of FMOC Glycine (American Peptide Company, Inc, Calif.), 1 equivalent of DIC and HOBT in DCM at room temperature for 4 hours. After deprotection, the product, HO-PEG-Glycine-NH₂, is purified by standard chromatographic techniques, and is then reacted with the peptide CRGDC combining one equivalent of each reactant using the methodology of Example 4.

Example 6

This example is directed to the preparation of CRGDC-percarboxylated branched PEG.

Branched PEG (4 Arm, 20 K, Shearwater Corporation) is reacted with 4 equivalents of chloroacetic acid and 8 equivalents of sodium hydroxide for 90-120 minutes at room temperature. The reaction is quenched by addition of sodium dihydrigephosphate and adjusting the pH to 7.0, and the resulting product, percarboxylated branched PEG, is purified by dialysis. The percarboxylated branched PEG is then coupled with the CRGDC peptide using the same coupling, cyclization, and isolation procedures as described in Examples 1 and 3.

Example 7

This example is directed to the preparation of PEG-PPG copolymers with pentaerythritol cores.

A. Branched block PEG-PPG copolymer with a pentaerythritol core.

Pentaerythritol (1 equiv.; Aldrich, 99+%, FW 136.15) is reacted with 4 equivalents of FMOC-PEG-NHS (Shearwater Corporation, MW 3400) in the presence of DIC in DCM. The reaction is allowed to proceed for 4 hours at room temperature, and the resulting precipitated dicyclohexyl urea is removed by filtration. The product is further purified by dialysis against distilled water to remove other unreacted reagents. The homogeneity is checked using reverse phase HPLC, and MS and IR are used to further characterize the product. The FMOC group is removed as described in Example 1, and the resulting material is then reacted with an excess of FMOC-PPG-NHS, as prepared in Example 3 (MW 3000), in the presence of DIC/HOBT to form the amide linkages. The reaction is carried out at room temperature for 4 to 8 hours. After deprotection, the product is first purified by dialysis using a membrane with a molecular weight cut-off of 5000. The product is then further purified by HPLC, and characterized by IR and MALDI Mass spectroscopy.

B. Branched PPG-PEG copolymer with a pentaerythritol core.

Pentaerythritol, (1 equiv.; Aldrich, 99+%, FW 136.15) is reacted with 4 equivalents of FMOC-PPG-NHS in the presence of DIC in DCM. The reaction is allowed to proceed for 4 hours at room temperature. The precipitated dicyclohexyl urea is removed by filtration, and the product is further purified by dialysis against distilled water to remove other unreacted reagents. The homogeneity is checked using reverse phase HPLC, and MS and IR is used to further characterize the product. The FMOC group is removed as described in Example 1, and the resulting material is then reacted with an excess of FMOC-PEG-NHS (Shearwater Corporation) (MW 3000) in the presence of DIC/HOBT to form the amide linkages. The reaction is carried out at room temperature for 4 to 8 hours, and the resulting product is first purified by dialysis using a membrane with a molecular weight cut off of 5000. The product is then further purified by HPLC, and characterized by IR and MALDI Mass spectroscopy.

Example 8

This example is directed to the preparation of PEG core with polylactide or polyglycolide arms.

In preparation for synthesis, polyglycolide (DuPont) and DL-polylactide (Aldrich) are freshly recrystallized from ethyl acetate. PEG oligomers of various molecular weights (Fluka or Polysciences) are dried under vacuum at 110° C. prior to use. Acryloyl chloride (Aldrich) is used as received. All other chemicals are of reagent grade and are used without further purification.

A. PEG with polyglycolide arms.

A 250 ml round bottom flask is flame dried under repeated cycles of vacuum and dry argon. PEG (20 g; molecular weight 10,000), 150 mL of xylene and 10 micrograms of stannous octoate are charged into the flask. The flask is heated to 60° C. under argon to dissolve the PEG, and cooled to room temperature. Polyglycolide (1.16 g) is added to the flask and the reaction mixture is refluxed for 16 hr. The resulting copolymer (10 K PEG-polyglycolide) is separated on cooling, recovered by filtration, and used directly as is in subsequent reactions.

B. PEG with polylactide arms.

PEG (MW 20,000) is dried by dissolving in benzene and distilling off the water as benzene azeotrope. In a glove bag, 32.43 g of PEG 20 k, 2.335 g of DL-polylactide and 15 mg of stannous octoate are charged into a 100 mL round bottom flask. The flask is capped with a vacuum stopcock, placed into a silicone oil bath and connected to a vacuum line. The temperature of the bath is raised to 200° C. The reaction is carried out for 4 hours at 200° C., after which the reaction mixture is cooled, dissolved in dichloromethane, and the copolymer is precipitated by pouring into an excess of dry ethyl ether. The copolymer is redissolved in 200 mL of dichloromethane in a 500 mL round bottom flask cooled to 0° C. To this flask are added 0.854 g of triethylamine and 0.514 mL of acryloyl chloride under a nitrogen atmosphere, and the reaction mixture is stirred for 12 hours at 0° C. The resulting triethylamine hydrochloride is separated by filtration and the copolymer is recovered from the filtrate by precipitating in diethyl ether. The polymer is dried at 50° C. under vacuum for 1 day.

Branched PEG may also be used to synthesize the corresponding polylactide and polyglcolide adducts. In these cases, the 4.64 g of polyglycolide and 8.34 g of DL-polylactide are used as reactants, respectively, to the molar equivalent of branched PEG from the procedures described above.

Example 9

This example is directed to the preparation of a pentaerythritol core with polylactide or polyglycolide arms.

Pentarythritol, (Aldrich, 99+%, FW 136.15) (1 equivalent) is reacted with 4 equivalents of polyglycolide in the presence of DIC in DCM. After the reaction proceeded for 4 hours at room temperature, the precipitated dicylohexyl urea is removed by filtration, and the resulting product is further purified by dialysis against distilled water to remove other unreacted reagents. Homogeneity is analyzed using reverse phase HPLC, and MS and IR are used to further characterize the product. The product has four equivalents of polyglycolide which are available for further derivatization, for example, with phosphorylated or percarboxylated branched PEG.

The above reaction is repeated using DL-polylactide to generate the corresponding polylactide derivative which may also be further derivatized with branched PEG. The resulting complexes contain a central core of penterythritol, 4 arms of polyglycolide or polylactide and terminal units of 10 Kd branched PEG.

Example 10

This example is directed to the preparation of an oligopeptide by recombinant methods.

The peptide GGGRGDS is produced by recombinant methods by intially synthesizing the DNA sequence GGC GGT GGG AGA GGA GAT AGT. This is cloned into a Cre recombinase based expression vector. Cre recombinase facilitates site-specific recombination at loxP sites, and recognizes and binds to inverted repeats that flank the spacer region where recombination occurs. The enzyme uses a reactive tyrosine within its active site to cleave the DNA in the spacer region, creating a staggered cut with sticky ends. Cre then reattaches the 5′ end of one loxP site to the 3′ end of the other loxP at the site of the staggered cut, thus recombining the DNA from two different vectors. Multiple reactions between the loxP site in pDNR and the two loxP sites in the acceptor vector occur simultaneously to transfer the gene and the chloramphenicol resistance gene into the acceptor vector. The plasmid is the Creator system available from Clontech (Palo Alto, Calif.). The acceptor vector in this case is an expression vector. The pTET-On (Clontech) vector expresses the exogenous gene in the presence of doxycycline. The vector is transferred into BL21-CodonPlus-RIL competent cells (Stratagene, La Jolla, Calif.). The genotype of these cells is strain^{a}: E. coli B F-ompT hsdS(rB-mB-) dcm+ Tet^{r} gal endA Hte [argU ileY leuW Cam^{r}]. These cells are protease deficient and designed for high-level protein expression from T7 RNA polymerase-based expression systems. Derived from E. coil B, these strains naturally lack the Lon protease and are engineered to be deficient for the OmpT protease. The Lon and OmpT proteases found in other E. coli expression hosts may interfere with the isolation of intact recombinant proteins.

The transformed cells are then grown in cell reactors to produce large quantities of GGGRGDS. The protein is extracted using the one-step bacterial protein extraction reagent B-PER (Pierce, Rockford, Ill.). After a complete protein extraction, the extract is run through an Ultralink Biosupport Medium affinity column with a bound peptide that binds GGGRGDS with high specificity (Pierce, Rockford, Ill.). After washing the column, the detergent concentration in the buffer is changed so that the GGGRGDS is released and collected.

B. Producing a Growth Factor by Recombinant Methods with Incorporation of Terminal Cys Residues into Mutagenized Growth Factor

The sequence for the basic fibroblast growth factor in humans is as follows:

1 aagcttcccc aaatctcctg cctccccacg ctgagttatc cgatgtctga aatgtcacag

61 cacttagtct tactcttcta tggcctactt tctactgcta tttgtgttac tcatgctacc

| | |
|---|---|
| 121 | catcttatct ccctcagtgt gtgagacgct ggcatcagat ttggcatctc ccacacactc |
| 181 | aacattatgt gttgcacaca gtaggtactc aatacatgca agttttctga atagatattt |
| 241 | tcctagtcat ctgtggcacc tgctatatcc tactgaaaat taccaaaatg caattaactt |
| 301 | caattttaca tttgggattt acagaaaata actctctctc caagaaatgc ataacaattt |
| 361 | agctagggca aatgccaggt ccgagttaag acattaatgc gcttcgatcg cgataaggat |
| 421 | ttatccttat ccccatcctc atctttctgc gtcgtctaat tcaagttagg tcagtaaagg |
| 481 | aaaccttttc gttttagcaa cccaatctgc tccccttctc tggcctcttt ctctcctttt |
| 541 | gttggtagac gacttcagcc tctgtccttt aattttaaag tttatgcccc acttgtaccc |
| 601 | ctcgtctttt ggtgatttag agattttcaa agcctgctct gacacagact cttccttgga |
| 661 | ttgcaacttc tctactttgg ggtggaaacg gcttctccgt tttgaaacgc tagcggggaa |
| 721 | aaaatggggg agaaagttga gtttaaactt ttaaaagttg agtcacggct ggttgcgcag |
| 781 | caaaagcccc gcagtgtgga gaaagcctaa acgtggtttg ggtggtgcgg gggttgggcg |
| 841 | ggggtgactt ttgggggata aggggcggtg gagcccaggg aatgccaaag ccctgccgcg |
| 901 | gcctccgacg cgcgcccccc gcccctcgcc tctcccccgc ccccgactga ggccgggctc |
| 961 | cccgccggac tgatgtcgcg cgcttgcgtg ttgtggccga accgccgaac tcagaggccg |
| 1021 | gccccagaaa acccgagcga gtagggggcg gcgcgcagga gggaggagaa ctgggggcgc |
| 1081 | gggaggctgg tgggtgtggg gggtggagat gtagaagatg tgacgccgcg gcccggcggg |
| 1141 | tgccagatta gcggacggtg cccgcggttg caacgggatc ccgggcgctg cagcttggga |
| 1201 | ggcggctctc cccaggcggc gtccgcggag acacccatcc gtgaacccca ggtcccgggc |
| 1261 | cgccggctcg ccgcgcacca ggggccggcg gacagaagag cggccgagcg gctcgaggct |
| 1321 | gggggaccgc gggcgcggcc gcgcgctgcc gggcgggagg ctggggggcc ggggccgggg |
| 1381 | ccgtgccccg gagcgggtcg gaggccgggg ccggggccgg gggacggcgg ctccccgcgc |
| 1441 | ggctccagcg gctcggggat cccggccggg ccccgcaggg accatggcag ccgggagcat |
| 1501 | caccacgctg cccgccttgc ccgaggatgg cggcagcggc gccttcccgc ccggccactt |
| 1561 | caaggacccc aagcggctgt actgcaaaaa cgggggcttc ttcctgcgca tccaccccga |
| 1621 | cggccgagtt gacggggtcc gggagaagag cgaccctcac agtgagtgcc gacccgctct |
| 1681 | ctccgcctca tttccatttc g |

The bFGF material is extracted from human cells in culture. The purified bFGF is then blunt end ligated to a linker peptide consisting of a repeat sequence of ACA (cysteine). The polymerase chain reaction method (PCR) is used to collect sufficient material. Two primers are designed with a melting temperature over 60° C., permitting the use of a higher annealing temperature in the PCR. The forward primer used is AGACATTAATGCGCTTCGATCG and the reverse primer is GGCGGAGTAAAGGTAAAGCTGA. The forward primer did not amplify the blunt end ligated section of ACA whereas the reverse primer did make that amplification. The PCR is carried out for 30 cycles with a 2 minute denaturation step at 95° C., a 30 second annealing step at 60° C. and a 3 minute extension step at 72° C. The Taq Polymerase enzyme used in the PCR is most efficient at polymerizing DNA at 72° C. This amplification program provides more than a million fold amplification of the DNA with a terminal cysteine added at the 3′ end. Sets of linkers and primers to add any of the amino acids at the 3′ terminus of this sequence are also prepared.

The bFGF sequence is cloned into the Creator system as described above. The cells are grown in a bacterial reactor, extracted using the B-PER procedure and then collected using an affinity column. In this case bFGF has a high affinity for Heparin sulfate. Heparin sulfate is immobilized using SulfoLink Coupling Gel columns (Pierce, Rockford, Ill.). The extraction column uses this affinity to bind the bFGF, and the buffer is changed after binding to release the bFGF protein for collection.

The mutagenized FGF containing a terminal cysteine is useful for preparing targeted polymers of the present invention. The terminal cysteine allows use of a maleimide linker to bind the protein to branched PEG. By first activating branched PEG to contain maleimide groups, the FGF is linked to the branched PEG as a bioconjugate. The maleimide reacts specifically with the sulfhydryl group of the cysteine when the pH is kept between 6.5 and 7.5. The modified bFGF is mixed with the maleimide substituted branched PEG at pH 7. The mixture is incubated overnight at room temperature to allow the binding to occur. The bound material is separated from the unbound material by fractionating in a size exclusion column packed with Sephadex G-75 (Sigma-Aldrich, St. Louis, Mo.).

Example 11

This example is directed to the preparation of a targeted polymeric composition of the present invention.

100 mg of a PEGylated phospholipid or branched PEG, 40 kD, Shearwater Polymers, Huntsville, Ala.) is dissolved in t-butanol (10 mL), and the resulting solution is heated over a 45-60° C. hot water bath and subjected to sonication until the solution clarifies. Tween 80 is added in a ratio from at least 1:5 to as much as 5:1 Tween 80:PEG component and sonication is applied again until the mixture clarifies. 10 mg of paclitaxel (Hauser Laboratories) is then added, followed by heating and sonication as above. The mixture is flash frozen over liquid nitrogen and lyophilized on an ice-water bath for 4 hours followed by room temperature overnight to remove t-butanol. The final lyophilisate may be optionally microfluidized at about 15,000 psi and then lyophilized again for storage. The dry powder so obtained may be rehydrated in 1.0 mL saline.

Example 12

The following example is directed to the preparation of nanoparticles comprising paclitaxel and a polymeric matrix comprising Tween (polysorbate).

955.6 mg of polyoyethylene-sorbitan monooleate (Tween 80) (Sigma Chemical Co. St. Louis Mo.) was dissolved in 30 mL of t-butanol in a round bottom flask at approximately 55° C. in a water bath with a rotor stirrer for approximately 20 min. This resulted in a clear solution to which 317.4 mg of paclitaxel (Natland International Corporation, N.C.) was added and dissolved under the same conditions. The flask was then immersed in liquid nitrogen (−78° C.) to flash-freeze the sample before it was lyophilized overnight (solvent trap temperature −45° C., pressure 7.0×10⁻³ mbar) to remove the residual solvent. Lyophilization yielded a yellow viscous liquid that was then hydrated with 20 mL of water. The hydrated material was dispersed using a microfluidizer, Model 110S, Microfluidics International Corp. (Newton, Mass.). The dispersion was translucent (<1 μm), had a pale-yellow tint, and showed no presence of crystals when inspected using a polarized light microscope. Sizing analysis revealed an average particle size of 63.0 nm.

Example 13

The following example is directed to the preparation of a targeted composition comprising camptothecin and a polymeric matrix comprising Tween (polysorbate).

A. 1.68 g of branched polyethylene glycol (bPEG), MW 20,000, 4 branches (Shearwater Polymers, Huntsville, Ala.) was dissolved in 30 mL of t-butanol in a round bottom flask at approximately 55° C. in a water bath with a rotor stirrer for approximately 20 min until the bPEG dissolved. This resulted in a clear solution to which 8.90 mg of camptothecin (Natland International Corporation, NC) and 10 mL of dichloromethane was added and dissolved with slight heating and exposure to ultrasound. The solution acquired a slight yellow tint after the camptothecin dissolved. Another 20 mL of t-butanol was added to the solution. The flask was then immersed in liquid nitrogen (−78° C.) to flash-freeze the sample prior to overnight lyophilization (solvent trap temperature −45° C., pressure 7.0×10⁻³ mbar) to remove the residual solvent. Lyophilization yielded a pale yellow flaky powder that was then hydrated with 20 mL of water. Water for hydration contained 303.8 mg (1% wt/vl) of polyoxyethylene-sorbitan monooleate (Tween 80). The hydrated material was dispersed using a microfluidizer, Model 110, Microfluidics International Corp. (Newton, Mass.). The dispersion was translucent (<1 μm), had a pale-yellow tint, and showed no presence of crystals when inspected using a polarized light microscope. The final concentration of the camptothecin in this particular formulation was 0.3 mg/mL. The same technique could be employed to increase the concentration up to 5.0 mg/mL.

Example 14

A. Pentaerythritol (Aldrich, 99+%, FW 136.15; 1 equivalent) is reacted with 3 equivalents of FMOC-PEG-NHS (Shearwater Corporation, MW 3400) in the presence of dicyclohexylcarbdiimide in DCM. The reaction is allowed to proceed for 4 hours at room temperature. The precipitated dicylohexyl urea is removed by filtration, and the resulting product is further purified by dialysis against distilled water to remove other unreacted reagents. The homogeneity is checked using reverse phase HPLC, and the resulting product, with three PEG arms, is reacted with stearic acid succinimide in the presence of DIC and HOBT for 4 hours in DCM. The resulting product is purified by dialysis and characterized by MS and IR spectroscopy.

B. The procedure from Step A may be modified to include a central PEG with two fatty acid arms or peptide arms, which may also include further units of PEG-amine for additional derivatization. A method derived from that of Clochard, et al., Macromol. Rapid Comm. (2000) 21:853-859 may also be used, in which bifunctional PEG-amine (NH-PEG-NH) is flanked in two hydrolytically labile amide linkages by groups which can be either peptides or proteins. The reaction starts with aminoethyl-terminated PEG and cis-aconitic hydride.

Example 15

The branched polymer of Example 9 is further derivatized with tissue plasminogen activator (t-PA) as described in Delgado C.,et al., Crit.Rev Ther Drug Carrier Sys,(1992) 9:249-304. The terminal —OH groups of the PEG are first activated with 1,1′-carbonyldiimidazole before addition of the t-PA.

Similarly, the reaction mPEG-OH+carbonylimidazole→mPEG-O—C(═O)-imidazole+R-NH₂→mPEG-O—C(═O)-NHR, where R is a protein with protected side chain amino groups, is an example of one of several means for coupling proteins to PEG. Harris, J. M., ed., “Polyethylene Glycol Chemistry. Biotechnical and Biomedical Applications,” Plenum Press, 1992.

Example 16

This example is directed to the preparation of biodegradable branched PEG (3 Arm).

PEG-2 Succinmide, MW 10,000 (Shearwater Corporation) is reacted with FMOC-aminoethyl ester of stearic acid in the presence of DIC and HOBT for 4 hours in DCM.

Example 17

Example 16 is repeated except methoxy PEG arms are substituted by FMOC-PEG by reacting FMOC-PEG-NHS ester with carboxy-protected lysine using techniques used for the synthesis of PEG-2 Succinimde.

Example 18

This example is directed to the preparation of N,N′-distearyldiaminobutryl-PEG3400-CRGDC (cyclic) using standard solid-phase techniques with Fmoc protecting groups.

A. Reagents

The reagents employed in this example are as follows:

20% piperidine in NMP (v/v) for removal of the Fmoc protecting groups.

Coupling agents: 1 M 1-hydroxybenzotriazole (HOBT) in NMP

1 M N,N′-diisopropylcarbodiimide (DIC) in NMP

Washing solvents: dichloromethane

methanol

Resin: Wang

Kaiser Reagents: Dilute 2 ml 1 mM aqueous KCN up to 100 ml with pyridine

500 mg ninhydrin in 10 ml absolute ethanol

80 g phenol in 20 ml absolute ethanol

A small amount of the peptide-resin was placed in a small test tube, and 2 drops of each solution above were added and placed in an oil bath for 2 minutes. Formation of a clear yellow solution indicated a strong negative reaction for primary amines, whereas a dark blue solution indicated a strong positive reaction for primary amines.

B. Procedure

The following procedure was employed, starting with the last amino acid in the peptide sequence attached to the resin.

The Fmoc protecting group was removed from the amino acid-resin using 20% piperidine/NMP solution. After waiting 20 minutes, the solution was tested for free amine groups using Kaiser (ninhydrin) reagents.

The resin was washed using alternating washes of dichloromethane and methanol (2× CH₂Cl₂, 2× CH₃OH, 2× CH₂Cl₂). To the washed resin was added 3 equivalents of the next amino acid in the sequence was added as a solid and 3 equivalents each of 1 M HOBT/NMP and 1 M DIC/NMP solutions. Sufficient NMP was added to cover the resin, and N₂ was bubbled up from the bottom of reaction vessel to stir. After stirring for approximately one hour, a small amount of the resin was removed from the reaction vessel using a disposable pipette and placed on a paper filter. After washing with methanol and dichloromethane as described above, a portion of the washed resin was used to perform the Kaiser test. Excess of the washed resin was returned to the reaction vessel. If the test was negative (i.e., yellow solution), excess reagents were washed from the resin using alternating dichloromethane and methanol washes. If the test was positive (i.e., blue solution), the reaction was allowed to continue. These steps were repeated with the next amino acid residue until the peptide sequence was complete.

After completion of the peptide sequence, the terminal Fmoc group from the last amino acid was removed with the piperidine solution. Solid Fmoc-NH-PEG3400-CO₂NHS (1 equivalent) with sufficient NMP to cover, followed by addition of 3 equivalents of HOBT/NMP and 1 M DIC/NMP. The reaction was allowed to proceed for 24 to 72 hours. Additional HOBT (solid) and DIC (neat) was added at approximately 24 hrs. After draining the reaction mixture, the resin was washed and dried over N₂. As 100% complete coupling is not achieved, the extent of coupling was determined by weight gain. This was capped with acetic anhydride and triethylamine before proceeding.

The Fmoc group was removed with piperidine solution. Analysis with Kaiser reagent revealed a positive Ninhydrin result. 3 equivalents of N-bis-Fmoc-L-2,4-diaminobutyric acid (Fmoc-Dab(Fmoc)-OH) and 3 equivalents of HOBT/NMP and DIC/NMP solutions were added, and the reaction was allowed to proceed for for 2 to 4 hours. When analysis with Kaiser reagent as described above provided a negative result, the reaction solution was filtered, and the resin was washed. The Fmoc group was removed with piperidine solution, and analysis with Kaiser reagent revealed a positive ninhydrin result.

Stearic acid (6 equivalents) was dissolved, with mild heating, in DMF, and the resulting solution was added to the reaction vessel. 6 eqs of solutions of HOBT/NMP and DIC/NMP solutions were added, and the reaction was allowed to proceed for several hours. Excess stearic acid was washed off, and analysis with Kaiser reagent indicated a positive ninhydrin result.

Resin was added with stirring to a solution of trifluoroacetic acid (TFA), ethanedithiol, phenol, thioanisol and water (8.3:0.25:0.5:0.5:0.5) ( v:v). The mixture was allowed to stir for 20 minutes, and the mixture was filtered through a coarse fritted funnel. The resin was washed with TFA and water, and the filtrate and washings were combined and the pH was adjusted to approx. pH 4.5 with aqueous 1 N NaOH. The solution was placed in dialysis tubing (MW 1000 cutoff) for initial purification in 20 L water.

The solution from the dialysis tubing was transferred to a beaker and the pH was adjusted to approximately pH 8 using 1 N NaOH and 30% (v) acetic acid, as necessary. 0.01 M aqueous K₃Fe(CN)₆ solution was added dropwise, with stirring, until a slight yellow color persisted. The pH was monitored and adjusted to near 8 using NaOH solution. It was observed that the rapidity of the pH change decreased when reaching the maximum amount of K₃Fe(CN)₆ solution. When the yellow color persisted, the pH was adjusted to 4.5-5 using 30% (v) acetic acid. Excess K₃Fe(CN)₆ was removed with AG-3 anion-exchange resin. The anion exchange resin was removed by filtration, and the filterate was placed in dialysis tubing (MW 1000 cutoff) for initial purification in 20 L water. The solution was transferred from the tubing to round bottom flasks and placed on a lyophilizer.

The lyophilized product was then dissolved in solvent and purified with a Vydac, TP-1010 C-18 reverse-phase column using an aqueous trifluoroacetic acid (TFA): methanol gradient. The purified product was characterized by MALDI mass spectrometry, NMR, and amino acid analysis.

Example 19

The final product from Example 18 is added to DPPE-PEG-5000 (Avanti Polar Lipids, Alabaster, Ala.) in a ratio of 9:1 mol/mol in t-butyl alcohol. Paclitaxel (10 mg) (Natural Pharmaceuticals, Boston, Mass.) is then added, and the resulting mixture is flash frozen and lyophilized to remove t-butanol. The dry powder is rehydrated in 1.0 ml saline.

Example 20

This example is directed to the preparation of Methoxy-PEG-decaleucine or Methoxy-PEG-decaisoleucine using standard solid-phase techniques with Fmoc protecting groups.

A. Reagents

The reagents described in Example 18 are also used in this example.

B. Procedure

Fmoc-Leu-OH or Fmoc-Ile-OH is coupled to the resin using methods described in commercial literature. The resin is swelled using alternating washes of dichloromethane and methanol (2× CH₂Cl₂, 2× CH₃OH, 2× CH₂C₂).

The Fmoc protecting group is removed from the amino acid-resin using 20% piperidine/NMP solution. After waiting 20 minutes, the solution is tested for free amine groups using Kaiser (ninhydrin) reagents.

The resin is washed using alternating washes of dichloromethane and methanol (2× CH₂Cl₂, 2× CH₃OH, 2× CH₂Cl₂). To the washed resin is added 3 equivalents of the next amino acid as a solid and 3 equivalents each of 1 M HOBT/NMP and 1 M DIC/NMP solutions. Sufficient NMP is added to cover the resin, and N₂ is bubbled up from the bottom of the reaction vessel to agitate, or by using a vortex mixer at 800 rpm. The mixture is stirred for approximately one hour and, if prepared by hand, a small amount of the resin is removed from the reaction vessel using a disposable pipette and placed on a paper filter. After washing with methanol and dichloromethane as described above, a portion of the washed resin is used to perform the Kaiser test. Excess of the washed resin is returned to the reaction vessel. If the test is negative (i.e., yellow solution), excess reagents are washed from the resin using alternating dichloromethane and methanol washes. If the test is positive (i.e., blue solution), the reaction is allowed to continue. If prepared on an automated synthesizer, the resin is washed after approximately 1 hour, without performing a Kaiser test. Any unreacted amine groups are capped using 5 drops each acetic anhydride and 5 drops triethylamine in DMF. This is allowed to react for 5 minutes, after which the solution is removed and the resin washed as previously described. These steps are repeated with the next amino acid residue until the peptide sequence is complete.

After completion of the peptide sequence, the terminal Fmoc group from the last amino acid is removed with the piperidine solution. The resin is dried to obtain a starting weight, and methoxy-PEG-succinimidyl propionate (mPEG-SPA; 1 equiv.), having a molecular weight of either 2000 or 5000, is added as a solid using sufficient NMP to cover, followed by addition of 3 equivalents of HOBT/NMP and 1 M DIC/NMP. The reaction is allowed to proceed for 24 to 72 hours. Additional HOBT (solid) and DIC (neat) is added at approximately 24 hrs. After draining the reaction mixture, while saving the PEG solution, the resin is washed and dried over N₂. As 100% complete coupling is not achieved, the extent of coupling is determined by weight gain. This is capped with acetic anhydride and triethylamine before proceeding.

Resin is added with stirring to a solution of 95% trifluoroacetic acid (TFA) in water (v/v). The mixture is allowed to stir for 20 minutes, and the mixture is filtered through a coarse fritted finnel. The resin is washed with TFA and water, and the filtrate and washings are combined and the pH adjusted to approx. pH 7 with aqueous 1 N NaOH. The solution is placed in dialysis tub|ing (MW 1000 cutoff) for initial purification in 20 L.

Example 21

This example is directed to the preparation of the following branched analog.

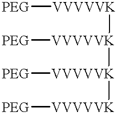

A. Reagents

The same reagents are used as in Examples 18 and 20.

B. Procedure (1) Preparation of Fmoc-PEG₃₄₀₀-VVVVV

Fmoc-Val-OH is coupled to the resin using methods described in commercial literature. The resin is swelled using alternating washes of dichloromethane and methanol (2× CH₂Cl₂, 2× CH₃OH, 2× CH₂Cl₂).

The Fmoc protecting group is removed from the amino acid-resin using 20% piperidine/NMP solution. After waiting 20 minutes, the solution is tested for free amine groups using Kaiser (ninhydrin) reagents.

The resin is washed using alternating washes of dichloromethane and methanol (2× CH₂Cl₂, 2× CH₃OH, 2× CH₂Cl₂). To the washed resin is added 3 equivalents of Fmoc-Val-OH as a solid and 3 equivalents each of 1 M HOBT/NMP and 1 M DIC/NMP solutions. Sufficient NMP is added to cover the resin, and N₂ is bubbled up from the bottom of the reaction vessel to agitate, or by using a vortex mixer at 800 rpm. The mixture is stirred for approximately one hour and, if prepared by hand, a small amount of the resin is removed from the reaction vessel using a disposable pipette and placed on a paper filter. After washing with methanol and dichloromethane as described above, a portion of the washed resin is used to perform the Kaiser test. Excess of the washed resin is returned to the reaction vessel. If the test is negative (i.e., yellow solution), excess reagents are washed from the resin using alternating dichloromethane and methanol washes. If the test is positive (i.e., blue solution), the reaction is allowed to continue. If prepared on an automated synthesizer, the resin is washed after approximately 1 hour, without performing a Kaiser test. Any unreacted amine groups are capped using 5 drops each acetic anhydride and 5 drops triethylamine in DMF. This is allowed to react for 5 minutes, after which the solution is removed and the resin washed as previously described. These steps are repeated with Fmoc-Val-OH until completion of a five amino acid peptide sequence.

After completion of the peptide sequence, the terminal Fmoc group from the last amino acid is removed with the piperidine solution. The resin is dried to obtain a starting weight, and Fmoc-PEG3400-CO₂NHS (1 equiv.), is added as a solid using sufficient NMP to cover, followed by addition of 3 equivalents of HOBT/NMP and 1 M DIC/NMP. The reaction is allowed to proceed for 24 to 72 hours. Additional HOBT (solid) and DIC (neat) is added at approximately 24 hrs. After draining the reaction mixture, while saving the PEG solution, the resin is washed and dried over N₂. As 100% complete coupling is not achieved, the extent of coupling is determined by weight gain. This is capped with acetic anhydride and triethylamine before proceeding.

Without removing the Fmoc group, resin is added with stirring to a solution of 95% trifluoroacetic acid (TFA) in water (v/v). The mixture is allowed to stir for 20 minutes, and the mixture is filtered through a coarse fritted funnel. The resin is washed with TFA and water, and the filtrate and washings are combined and the pH adjusted to approx. pH 7 with aqueous 1 N NaOH. The solution is placed in dialysis tubing (MW 1000 cutoff) for initial purification in 20 L.

(2) Preparation of Fmoc-KKK-Wang resin

Fmoc-Lys(Dde)-OH is coupled to the resin using methods described in commercial literature. The resin is swelled using alternating washes of dichloromethane and methanol (2× CH₂Cl₂, 2× CH₃OH, 2× CH₂Cl₂).

The Fmoc protecting group is removed from the amino acid-resin using 20% piperidine/NMP solution. After waiting 20 minutes, the solution is tested for free amine groups using Kaiser (ninhydrin) reagents.

The resin is washed using alternating washes of dichloromethane and methanol (2× CH₂Cl₂, 2× CH₃OH, 2× CH₂Cl₂). To the washed resin is added 3 equivalents of Fmoc-Lys(Dde)-OH as a solid and 3 equivalents each of 1 M HOBT/NMP and 1 M DIC/NMP solutions. Sufficient NMP is added to cover the resin, and N₂ is bubbled up from the bottom of the reaction vessel to agitate, or by using a vortex mixer at 800 rpm. The mixture is stirred for approximately one hour and, if prepared by hand, a small amount of the resin is removed from the reaction vessel using a disposable pipette and placed on a paper filter. After washing with methanol and dichloromethane as described above, a portion of the washed resin is used to perform the Kaiser test. Excess of the washed resin is returned to the reaction vessel. If the test is negative (i.e., yellow solution), excess reagents are washed from the resin using alternating dichloromethane and methanol washes. If the test is positive (i.e., blue solution), the reaction is allowed to continue. If prepared on an automated synthesizer, the resin is washed after approximately 1 hour, without performing a Kaiser test. Any unreacted amine groups are capped using 5 drops each acetic anhydride and 5 drops triethylamine in DMF. This is allowed to react for 5 minutes, after which the solution is removed and the resin washed as previously described. These steps are repeated with Fmoc-Lys(Dde)-OH until completion of a four amino acid peptide sequence (i.e., Fmoc-(K(Dde))₄-Wang).

After completion of the peptide sequence, the Dde protecting groups are removed from the Lysines using 2% hydzine in DMF. The reaction mixture is stirred at room temperature for 3 minutes, after which the resin is filtered and the hydrazine treatment is repeated two more times. The resin is washed with DMF and alternating washes of dichloromethane and methanol. The presence of free amines is checked using the Kaiser test, and the number of free amines is quantified using the Kaiser test.

(3) Preparation of Final Branched Analog

Fmoc-PEG-VVVVV-CO₂NHS is coupled to Fmoc-KKKK-Wang using 12 equivalents with 12 equivalents each of 1 M HOBT/NMP and 1 M DIC/NMP. The reaction is stirred under N₂, and the Kaiser test is used to monitor the reaction for completeness. Once the Kaiser test is negative, the resin is washed using dichloromethane and methanol, and the the Fmoc protecting group is removed from the amino acid-resin using 20% piperidine/NMP solution. After waiting 20 minutes, the solution is tested for free amine groups using Kaiser (ninhydrin) reagents. The resin is washed using alternating washes of dichloromethane and methanol.

Resin is added with stirring to a solution of 95% trifluoro acetic acid (TFA) in water (v/v). The mixture is allowed to stir for 20 minutes, and the mixture is filtered through a coarse fritted funnel. The resin is washed with TFA and water, and the filtrate and washings are combined and the pH adjusted to approx. pH 7 with aqueous 1 N NaOH. The solution is placed in dialysis tubing (MW 1000 cutoff) for initial purification in 20 L.

B′. Alternate Procedure

The following is an alternate procedure for preparing the branched analog set forth above.

Dde-Lys(Fmoc)-OH is coupled to the resin using methods described in commercial literature. The resin is swelled using alternating washes of dichloromethane and methanol (2× CH₂Cl₂, 2× CH₃OH, 2× CH₂Cl₂).

The Fmoc protecting group is removed from the amino acid-resin using 20% piperidine/NMP solution. After waiting 20 minutes, the solution is tested for free amine groups using Kaiser (ninhydrin) reagents.

To the resin are added 3 equivalents of Fmoc-Val-OH and 3 equivalents each of 1 M HOBT/NMP and 1 M DIC/NMP solutions. Sufficient NMP is added to cover the resin, and N₂ is bubbled up from the bottom of the reaction vessel to agitate, or by using a vortex mixer at 800 rpm. The mixture is stirred for approximately one hour and, if prepared by hand, a small amount of the resin is removed from the reaction vessel using a disposable pipette and placed on a paper filter. After washing with methanol and dichloromethane as described above, a portion of the washed resin is used to perform the Kaiser test. Excess of the washed resin is returned to the reaction vessel. If the test is negative (i.e., yellow solution), excess reagents are washed from the resin using alternating dichloromethane and methanol washes. If the test is positive (i.e., blue solution), the reaction is allowed to continue. These steps are repeated with Fmoc-Val-OH until completion of a six amino acid peptide sequence (i.e., Dde-K(Fmoc-VVVVV)-Wang).

After completion of the peptide sequence, the terminal Fmoc group is removed from the last valine with the piperidine solution. The resin is dried to obtain a starting weight, and methoxy-PEG-succinimidyl propionate (mPEG-SPA) (1 equiv.), having a molecular weight of 2000 or 5000, is added as a solid using sufficient NMP to cover, followed by addition of 3 equivalents of HOBT/NMP and 1 M DIC/NMP. The reaction is allowed to proceed for 24 to 72 hours. Additional HOBT (solid) and DIC (neat) is added at approximately 24 hrs. After draining the reaction mixture, while saving the PEG solution, the resin is washed and dried over N₂. As 100% complete coupling is not achieved, the extent of coupling is determined by weight gain. This is capped with acetic anhydride and triethylamine before proceeding.

The resin is divided and a portion of which is set aside for later use. To cleave the Dde-K(methoxy-PEG-VVVVV) from the resin, resin is added with stirring to a solution of 95% trifluoroacetic acid (TFA) in water (v/v). The mixture is allowed to stir for 20 minutes, and the mixture is filtered through a coarse fritted funnel. The resin is washed with TFA and water, and the filtrate and washings are combined and the pH adjusted to approx. pH 7 with aqueous 1 N NaOH. The solution is placed in dialysis tubing (MW 1000 cutoff) for initial purification in 20 L. The volume of the resulting mixture is reduced, and the mixture is placed on a lyophilizer until a dry powder is obtained, which is subsequently purified using HPLC.

The Dde protecting groups are removed from the retained Dde-K(methoxy-PEG-VVVVV) using 2% hydzine in DMF. The reaction mixture is stirred at room temperature for 3 minutes, after which the resin is filtered and the hydrazine treatment is repeated two more times. The resin is washed with DMF and alternating washes of dichloromethane and methanol. The presence of free amines is checked using the Kaiser test, and the number of free amines is quantified using the Kaiser test.

Dde-K(methoxy-PEG-VVVVV) is coupled to the deprotected K(methoxy-PEG-VVVVV) using 3 equivalents with 3 equivalents each of 1 M HOBT/NMP and 1 M DIC/NMP. Sufficient NMP is added to cover the resin, and N₂ is bubbled up from the bottom of the reaction vessel to agitate, or by using a vortex mixer at 800 rpm. The mixture is stirred for approximately one hour and, if prepared by hand, a small amount of the resin is removed from the reaction vessel using a disposable pipette and placed on a paper filter. After washing with methanol and dichloromethane as described above, a portion of the washed resin is used to perform the Kaiser test. Excess of the washed resin is returned to the reaction vessel. If the test is negative (i.e., yellow solution), excess reagents are washed from the resin using alternating dichloromethane and methanol washes. If the test is positive (i.e., blue solution), the reaction is allowed to continue. These steps are repeated to form the final compound.

The fmal compound is cleaved from the resin using a solution of 95% trifluoroacetic acid (TFA) in water (v/v). The mixture is allowed to stir for 20 minutes, and the mixture is filtered through a coarse fritted funnel. The resin is washed with TFA and water, and the filtrate and washings are combined and the pH adjusted to approx. pH 7 with aqueous 1 N NaOH. The solution is placed in dialysis tubing (MW 1000 cutoff) for initial purification in 20 L. The final product is then purified using HPLC.

Example 22

This example is directed to the preparation of CRGDS-PEG-LLLLLLLLLL using standard solid-phase techniques with Fmoc protecting groups.

A. Reagents

The same reagents are used as in Examples 18, 20 and 21.

B. Procedure

Fmoc-Leu-OH is coupled to the resin using methods described in the commercial literature. The resin is swelled using alternating washes of dichloromethane and methanol (2× CH₂Cl₂, 2× CH₃OH, 2× CH₂Cl₂).

The Fmoc protecting group is removed from the amino acid-resin using 20% piperidine/NMP solution. After waiting 20 minutes, the solution is tested for free amine groups using Kaiser (ninhydrin) reagents.

The resin is washed using alternating washes of dichloromethane and methanol (2× CH₂Cl₂, 2× CH₃OH, 2× CH₂Cl₂). To the washed resin is added 3 equivalents of Fmoc-Lys(Dde)-OH as a solid and 3 equivalents each of 1 M HOBT/NMP and 1 M DIC/NMP solutions. Sufficient NMP is added to cover the resin, and N₂ is bubbled up from the bottom of the reaction vessel to agitate, or by using a vortex mixer at 800 rpm. The mixture is stirred for approximately one hour and, if prepared by hand, a small amount of the resin is removed from the reaction vessel using a disposable pipette and placed on a paper filter. After washing with methanol and dichloromethane as described above, a portion of the washed resin is used to perform the Kaiser test. Excess of the washed resin is returned to the reaction vessel. If the test is negative (i.e., yellow solution), excess reagents are washed from the resin using alternating dichloromethane and methanol washes. If the test is positive (i.e., blue solution), the reaction is allowed to continue. If prepared on an automated synthesizer, the resin is washed after approximately 1 hour, without performing a Kaiser test. Any unreacted amine groups are capped using 5 drops each acetic anhydride and 5 drops triethylamine in DMF. This is allowed to react for 5 minutes, after which the solution is removed and the resin washed as previously described. These steps are repeated with the next amino acid residue until completion of the decaleucine peptide sequence (i.e., Fmoc-(L)₁₀-OH).

After completion of the peptide sequence, the terminal Fmoc group from the last amino acid is removed with the piperidine solution. Solid Fmoc-NH-PEG3400-CO₂NHS (1 equivalent) is added with sufficient NMP to cover, followed by addition of 3 equivalents of HOBT/NMP and 1 M DIC/NMP. The reaction is allowed to proceed for 24 to 72 hours. Additional HOBT (solid) and DIC (neat) is added at approximately 24 hrs. After draining the reaction mixture, the resin is washed and dried over N₂. As 100% complete coupling is not achieved, the extent of coupling is determined by weight gain. This is capped with acetic anhydride and triethylamine before proceeding.

The Fmoc protecting group is removed from the amino acid-resin using 20% piperidine/NMP solution. After waiting 20 minutes, the solution is tested for free amine groups using Kaiser (ninhydrin) reagents. 3 equivalents of Fmoc-Cys(trt)-OH as a solid and 3 equivalents each of 1 M HOBT/NMP and 1 M DIC/NMP solutions are added. Sufficient NMP is added to cover the resin, and N₂ is bubbled up from the bottom of the reaction vessel to agitate, or by using a vortex mixer at 800 rpm. The mixture is stirred for approximately one hour and, if prepared by hand, a small amount of the resin is removed from the reaction vessel using a disposable pipette and placed on a paper filter. After washing with methanol and dichloromethane as described above, a portion of the washed resin is used to perform the Kaiser test. Excess of the washed resin is returned to the reaction vessel. If the test is negative (i.e., yellow solution), excess reagents are washed from the resin using alternating dichloromethane and methanol washes. If the test is positive (i.e., blue solution), the reaction is allowed to continue. If prepared on an automated synthesizer, the resin is washed after approximately 1 hour, without performing a Kaiser test. Any unreacted amine groups are capped using 5 drops each acetic anhydride and 5 drops triethylamine in DMF. This is allowed to react for 5 minutes, after which the solution is removed and the resin washed as previously described.

The Fmoc protecting group is removed with 20% piperidine solution and the previous steps are repeated with Fmoc-Asp(OtBu)-OH, Fmoc-Gly-OH, Fmoc-Arg(pbf)-OH and finally with Fmoc-Cys(trt)-OH to complete the series. The Fmoc group from the terminal Cys is removed using 20% piperidine in NMP solution, and the resulting material is washed with alternating aliquots of dichloromethane and methanol.

The resin is added with stirring to a solution of trifluoroacetic acid (TFA), ethanedithiol, phenol, thioanisol and water (8.3:0.25:0.5:0.5:0.5) (v:v). The mixture is allowed to stir for 20 minutes, and the mixture is filtered through a coarse fritted finnel. The resin is washed with TFA and water, and the filtrate and washings are combined and the pH adjusted to approx. pH 4.5 with aqueous 1 N NaOH. The solution is placed in dialysis tubing (MW 1000 cutoff) for initial purification in 20 L.

For cyclization, the solution from the dialysis tubing is transferred to a beaker and the pH is adjusted to approximately pH 8 using 1 N NaOH and 30% (v) acetic acid if necessary. While stirring, aqueous K₃Fe(CN)₆ solution (0.01 M) is added dropwise until a slight yellow color persists. The pH is monitored to maintain near pH 8, using a NaOH solution to adjust, as needed. The rapidity of the pH change diminishes when nearing the maximum amount of K₃Fe(CN)₆ solution. When the yellow color persists, the pH is adjusted to pH 4.5 to 5 using 30% (v/v) acetic acid. Excess K₃Fe(CN)₆ is removed with AG-3 anion-exchange resin, and the filtrate is filtered to remove the anion exchange resin. The filtrate is placed in dialysis tubing (MW 1000 cutoff) for initial purification in 20 L water, and the solution is transferred from the tubing to round bottomed flasks and placed on the lyophilizer. The lyophilized product is then dissolved in a suitable solvent and purified with a Vydac, TP-1010 C-18 reverse-phase column using an aqueous trifluoroacetic acid (TFA): methanol gradient. The purified product is characterized by MALDI mass spectrometry, NMR, and amino acid analysis.

The disclosures of each patent, patent application and publication cited or described in this document are hereby incorporated by reference, in their entirety.

Various modifications of the invention, in addition to those described herein, will be apparent to those skilled in the art from the foregoing description. Such modifications are also intended to fall within the scope of the appended claims.

What is claimed is:

1. A pharmaceutical composition comprising, in combination with an effective amount of a bioactive agent, a targeted matrix which comprises a polymer and a targeting ligand, wherein said targeting ligand is covalently associated with said polymer and said bioactive agent is associated non-covalently with said polymer, and wherein said bioactive agent is substantially homogeneously dispersed throughout said matrix.

2. A pharmaceutical composition according to claim 1 wherein said polymer comprises repeating alkylene groups, wherein each alkylene group optionally contains from one to three heteroatoms selected from —O—, -N(R)- or —S(O)ₙ—, where R is hydrogen or alkyl and n is 0 to about 1000.

3. A pharmaceutical composition according to claim 2 wherein said polymer is selected from the group consisting of a polyalkylene oxide, polyalkyleneimine, polyalkylene amine, polyalkene sulfide, polyalkylene sulfonate, polyalkylene sulfone, poly(alkylenesulfonylalkyleneimine) and copolymers thereof.

4. A pharmaceutical composition according to claim 3 wherein said polymer is selected from the group consisting of a polyethylene glycol, polypropylene glycol, branched polyethylene imine, polyvinyl pyrrolidone, polylactide, poly(lactide-co-glycolide), polysorbate, polyethylene oxide, poly(ethylene oxide-co-propylene oxide), poly(oxyethylated) glycerol, poly(oxyethylated) sorbitol, poly(oxyethylated glucose), polymethyloxazoline, polyethyloxazoline, polyhydroxyethyloxazoline, polyhydroxypropyloxazoline, polyvinyl alcohol, poly(hydroxyalkylcarboxylic acid), polyhydroxyethyl acrylic acid, polyhydroxypropyl methacrylic acid, polyhydroxyvalerate, polyhydroxybutyrate, polyoxazolidine, polyaspartamide, polysialic acid, linear polypropylene imine, polyethylene sulfide, polypropylene sulfide, polyethylenesulfonate, polypropylenesulfonate, polyethylene sulfone, polyethylenesulfonylethyleneimine, polycaprolactone, polypropylene oxide, polyvinylmethylether, polyhydroxyethyl acrylate, polyhydroxypropyl methacrylate, polyphosphazene and derivatives, mixtures and copolymers thereof.

5. A pharmaceutical composition according to claim 4 wherein said polymer is selected from the group consisting of a polyethylene glycol and polypropylene glycol and copolymers thereof.

6. A pharmaceutical composition according to claim 5 wherein said polymer is polyethylene glycol.

7. A pharmaceutical composition according to claim 1 wherein said polymer comprises a polypeptide.

8. A pharmaceutical composition according to claim 1 wherein said bioactive agent is an anti-cancer agent.

9. A pharmaceutical composition according to claim 8 wherein said anti-cancer agent is selected from the group consisting of paclitaxel, docetaxel, camptothecin, and derivatives and analogs thereof.

10. A pharmaceutical composition according to claim 9 wherein said anti-cancer agent is paclitaxel.

11. A pharmaceutical composition according to claim 9 wherein said anti-cancer agent is docetaxel.

12. A pharmaceutical composition according to claim 9 wherein said anti-cancer agent is camptothecin.

13. A pharmaceutical composition according to claim 1 wherein said bioactive agent has limited water solubility.

14. A pharmaceutical composition according to claim 13 wherein the ratio of the solubility of said bioactive agent in said polymer to the solubility of said bioactive agent in water is greater than about 1:1.

15. A pharmaceutical composition according to claim 14 wherein said ratio is at least about 10:1.

16. A pharmaceutical composition according to claim 1 wherein said targeting ligand targets cells or receptors associated with diseased tissue.

17. A pharmaceutical composition according to claim 1 wherein said targeting ligand is selected from the group consisting of proteins, peptides, cytokines, growth factors, vitamins, vitamin analogues, polysaccharides, glycopeptides, glycoproteins, steroids, steroid analogs, hormones, cofactors, bioactive agents, genetic material, drug molecules, and antagonists of the GPIIBIIIA receptor of platelets.

18. A pharmaceutical composition according to claim 17 wherein said targeting ligand is selected from the group consisting of proteins and peptides.

19. A pharmaceutical composition according to claim 18 wherein said targeting ligand comprises a peptide.

20. A pharmaceutical composition according to claim 19 wherein said peptide is selected from the group consisting of linear peptides and cyclized peptides.

21. A pharmaceutical composition according to claim 19 wherein said peptide targets cells or receptors associated with tissue selected from the group consisting of brain, kidney, lung, skin, pancreas, intestine, uterus, adrenal gland and retina tissue.

22. A pharmaceutical composition according to claim 21 wherein said peptide targets cells or receptors associated with brain tissue.

23. A pharmaceutical composition according to claim 22 wherein said peptide comprises a sequence selected from the group consisting of CNSRLHLRC, CENWWGDVC, WRCVLREGPAGGCAWFNRHRL, and CLSSRLDAC.

24. A pharmaceutical composition according to claim 21 wherein said peptide targets cells or receptors associated with kidney tissue.

25. A pharmaceutical composition according to claim 24 wherein said peptide comprises a sequence selected from the group consisting of CLPVASC and CGAREMC.

26. A pharmaceutical composition according to claim 21 wherein said peptide targets cells or receptors associated with lung tissue.

27. A pharmaceutical composition according to claim 26 wherein said peptide comprises a sequence selected from the group consisting of CGFECVRQCPERC, CGFELETC, CTLRDRNC and CIGEVEVC.

28. A pharmaceutical composition according to claim 21 wherein said peptide targets cells or receptors associated with skin tissue.

29. A pharmaceutical composition according to claim 28 wherein said peptide comprises the sequence CVALCREACGEGC.

30. A pharmaceutical composition according to claim 21 wherein said peptide targets cells or receptors associated with pancreas tissue.

31. A pharmaceutical composition according to claim 30 wherein said peptide comprises the sequence SWCEPGWCR.

32. A pharmaceutical composition according to claim 21 wherein said peptide targets cells or receptors associated with intestinal tissue.

33. A pharmaceutical composition according to claim 32 wherein said peptide comprises the sequence YSGKWGW.

34. A pharmaceutical composition according to claim 21 wherein said peptide targets cells or receptors associated with uterine tissue.

35. A pharmaceutical composition according to claim 34 wherein said peptide comprises the sequence GLSGGRS.

36. A pharmaceutical composition according to claim 21 wherein said peptide targets cells or receptors associated with adrenal gland tissue.

37. A pharmaceutical composition according to claim 36 wherein said peptide comprises the sequence LMLPRAD.

38. A pharmaceutical composition according to claim 21 wherein said peptide targets cells or receptors associated with retinal tissue.

39. A pharmaceutical composition according to claim 38 wherein said peptide comprises a sequence selected from the group consisting of CRDVVSVIC and CSCFRDVCC.

40. A pharmaceutical composition according to claim 19 wherein said peptide inhibits integrin-expressing cells from binding to extracellular matrix proteins.

41. A pharmaceutical composition according to claim 40 wherein said peptide inhibits the binding of fibronectin to α5-β1 integrin.

42. A pharmaceutical composition according to claim 41 wherein said peptide comprises a sequence selected from the group consisting of CRGDC, CRGDCL, NGR(AHA), DGR(AHA), CRGDCA, RCDVVV, SLIDIP, TIRSVD, KRGD, RRGP and RGDL.

43. A pharmaceutical composition according to claim 19 wherein said peptide foms RGD-type binding determinants of antibodies.

44. A pharmaceutical composition according to claim 43 wherein said peptide is selected from the group consisting of CSFGRGDIRNC, CSFGRTDQRIC, CSFGKGDNRIC, CSFGRNDSRNC, CSFGRVDDRNC, CSFGRADRRNC, CSFGRSVDRNC, CSFGKRDMRNC, CSFGRWDARNC, CSFGRQDVRNC and CSFGRDDGRN

45. A pharmaceutical composition according to claim 19 wherein said peptide targets angiogenic endothelium associated with solid tumors.

46. A pharmaceutical composition according to claim 45 wherein said peptide comprises a sequence selected from the group consisting of CDCRGDCFC and CNGRCVSGCAGRC.

47. A pharmaceutical composition according to claim 19 wherein said peptide targets receptors associated with cancer cells.

48. A pharmaceutical composition according to claim 47 wherein said peptide is selected from the group consisting of Abaecins, Andropins, Apidaecins, AS, Bactenecins, Bac, Bactericidins, Bacteriocins, Bombinins, Bombolitins, BPTI, Brevinins, Cecropins, Charybdtoxins, Coleoptericins, Crabolins, α-Defensins, β-Defensins, Defensins-insect, Defensins-scorpion, Dermaseptins, Diptericins, Drosocins, Esculentins, Indolicidins, Lactoferricins, Lantibiotics, Leukocons, Magainins, Mastoparans, Melittins, Phormicins, Polyphemusins, Protegrins, Royalisins, Sarcotoxins, Seminal Plasmins, Tachyplesins, Thionins and Toxins.

49. A targeted matrix for use as a delivery vehicle for a bioactive agent, wherein the matrix comprises a polymer that is covalently associated with a targeting ligand.

50. A targeted matrix according to claim 49 which has a morphology selected from the group consisting of particulate and micellar.

51. A targeted matrix according to claim 50 which has a particulate morphology.

52. A targeted matrix according to claim 51 wherein said particles have a diameter of from about 1 nm to about 1000 nm.

53. A targeted matrix according to claim 52 wherein said particles have a diameter of from about 10 nm to about 500 nm.

54. A targeted matrix according to claim 53 wherein said particles have a diameter of from about 20 nm to about 200 nm.

55. A targeted matrix according to claim 49 wherein said polymer comprises repeating alkylene groups, wherein each alkylene group optionally contains from one to three heteroatoms selected from —O—, -N(R)- or —S(O)ₙ—, where R is hydrogen or alkyl and n is 0 to about 1000.

56. A targeted matrix according to claim 55 wherein said polymer is selected from the group consisting of a polyalkylene oxide, polyalkyleneimine, polyalkylene amine, polyalkene sulfide, polyalkylene sulfonate, polyalkylene sulfone, poly(alkylenesulfonylalkyleneimine) and copolymers thereof.

57. A pharmaceutical composition according to claim 56 wherein said polymer is selected from the group consisting of a polyethylene glycol, polypropylene glycol, branched polyethylene imine, polyvinyl pyrrolidone, polylactide, poly(lactide-co-glycolide), polysorbate, polyethylene oxide, poly(ethylene oxide-co-propylene oxide), poly(oxyethylated) glycerol, poly(oxyethylated) sorbitol, poly(oxyethylated glucose), polymethyloxazoline, polyethyloxazoline, polyhydroxyethyloxazoline, polyhydroxypropyloxazoline, polyvinyl alcohol, poly(hydroxyalkylcarboxylic acid), polyhydroxyethyl acrylic acid, polyhydroxypropyl methacrylic acid, polyhydroxyvalerate, polyhydroxybutyrate, polyoxazolidine, polyaspartamide, polysialic acid, linear polypropylene imine, polyethylene sulfide, polypropylene sulfide, polyethylenesulfonate, polypropylenesulfonate, polyethylene sulfone, polyethylenesulfonylethyleneimine, polycaprolactone, polypropylene oxide, polyvinylmethylether, polyhydroxyethyl acrylate, polyhydroxypropyl methacrylate, polyphosphazene and derivatives, mixtures and copolymers thereof.

58. A targeted matrix according to claim 57 wherein said polymer is selected from the group consisting of a polyethylene glycol and polypropylene glycol and copolymers thereof.

59. A targeted matrix according to claim 49 wherein said polymer is selected from the group consisting of linear, branched and star structures.

60. A targeted matrix according to claim 59 wherein said polymer is a branched structure.

61. A targeted matrix according to claim 60 wherein said branched structure comprises from about 4 to about 10 arms.

62. A targeted matrix according to claim 61 wherein said branched structure comprises from about 4 to about 8 arms.

63. A targeted matrix according to claim 59 wherein said polymer has a star structure.

64. A targeted matrix according to claim 63 wherein said star structure comprises from about 3 to about 12 arms.

65. A targeted matrix according to claim 64 wherein said star structure comprises from about 4 to about 8 arms.7.

66. A targeted matrix according to claim 49 wherein said polymer comprises a polypeptide.

67. A targeted matrix according to claim 49 wherein said targeting ligand is selected from the group consisting of proteins, peptides, cytokines, growth factors, vitamins, vitamin analogues, polysaccharides, glycopeptides, glycoproteins, steroids, steroid analogs, hormones, cofactors, bioactive agents, genetic material, drug molecules, and antagonists of the GPIIBIIIA receptor of platelets.

68. A targeted matrix according to claim 67 wherein said targeting ligand is selected from the group consisting of proteins and peptides.

69. A targeted matrix according to claim 68 wherein said targeting ligand comprises a peptide.

70. A targeted matrix according to claim 69 wherein said peptide is selected from the group consisting of linear peptides and cyclized peptides.

71. A targeted matrix according to claim 69 wherein said peptide targets cells or receptors associated with tissue selected from the group consisting of brain, kidney, lung, skin, pancreas, intestine, uterus, adrenal gland and retina tissue.

72. A targeted matrix according to claim 71 wherein said peptide targets cells or receptors associated with brain tissue.

73. A targeted matrix according to claim 72 wherein said peptide comprises a sequence selected from the group consisting of CNSRLHLRC, CENWWGDVC, WRCVLREGPAGGCAWFNRHRL, and CLSSRLDAC.

74. A targeted matrix according to claim 71 wherein said peptide targets cells or receptors associated with kidney tissue.

75. A targeted matrix according to claim 74 wherein said peptide comprises a sequence selected from the group consisting of CLPVASC and CGAREMC.

76. A targeted matrix according to claim 71 wherein said peptide targets cells or receptors associated with lung tissue.

77. A targeted matrix according to claim 76 wherein said peptide comprises a sequence selected from the group consisting of CGFECVRQCPERC, CGFELETC, CTLRDRNC and CIGEVEVC.

78. A targeted matrix according to claim 71 wherein said peptide targets cells or receptors associated with skin tissue.

79. A targeted matrix according to claim 78 wherein said peptide comprises the sequence CVALCREACGEGC.

80. A targeted matrix according to claim 71 wherein said peptide targets cells or receptors associated with pancreas tissue.

81. A targeted matrix according to claim 80 wherein said peptide comprises the sequence SWCEPGWCR.

82. A targeted matrix according to claim 71 wherein said peptide targets cells or receptors associated with intestinal tissue.

83. A targeted matrix according to claim 82 wherein said peptide comprises the sequence YSGKWGW.

84. A targeted matrix according to claim 71 wherein said peptide targets cells or receptors associated with uterine tissue.

85. A targeted matrix according to claim 84 wherein said peptide comprises the sequence GLSGGRS.

86. A targeted matrix according to claim 71 wherein said peptide targets cells or receptors associated with adrenal gland tissue.

87. A targeted matrix according to claim 86 wherein said peptide comprises the sequence LMLPRAD.

88. A targeted matrix according to claim 71 wherein said peptide targets cells or receptors associated with retinal tissue.

89. A targeted matrix according to claim 88 wherein said peptide comprises a sequence selected from the group consisting of CRDVVSVIC and CSCFRDVCC.

90. A targeted matrix according to claim 71 wherein said peptide inhibits integrin-expressing cells from binding to extracellular matrix proteins.

91. A targeted matrix according to claim 90 wherein said peptide inhibits the binding of fibronectin to α5-β1 integrin.

92. A targeted matrix according to claim 91 wherein said peptide is selected from the group consisting of CRGDC, CRGDCL, NGR(AHA), DGR(AHA), CRGDCA, RCDVVV, SLIDIP, TIRSVD, KRGD, RRGP and RGDL.

93. A targeted matrix according to claim 69 wherein said peptide forms RGD-type binding determinants of antibodies.

94. A targeted matrix according to claim 93 wherein said peptide is selected from the group consisting of CSFGRGDIRNC, CSFGRTDQRIC, CSFGKGDNRIC, CSFGRNDSRNC, CSFGRVDDRNC, CSFGRADRRNC, CSFGRSVDRNC, CSFGKRDMRNC, CSFGRWDARNC, CSFGRQDVRNC and CSFGRDDGRNC.

95. A targeted matrix according to claim 49 wherein said peptide targets angiogenic endothelium associated with solid tumors.

96. A targeted matrix according to claim 95 wherein said peptide comprises a sequence selected from the group consisting of CDCRGDCFC and CNGRCVSGCAGR

97. A targeted matrix according to claim 49 wherein said peptide targets receptors associated with cancer cells.

98. A targeted matrix according to claim 97 wherein said peptide is selected from the group consisting of Abaecins, Andropins, Apidaecins, AS, Bactenecins, Bac, Bactericidins, Bacteriocins, Bombinins, Bombolitins, BPTI, Brevinins, Cecropins, Charybdtoxins, Coleoptericins, Crabolins, α-Defensins, β-Defensins, Defensins-insect, Defensins-scorpion, Dermaseptins, Diptericins, Drosocins, Esculentins, Indolicidins, Lactoferricins, Lantibiotics, Leukocons, Magainins, Mastoparans, Melittins, Phormicins, Polyphemusins, Protegrins, Royalisins, Sarcotoxins, Seminal Plasmins, Tachyplesins, Thionins and Toxins.

99. A method for enhancing the bioavailability of a bioactive agent in vivo comprising (i) providing a pharmaceutical composition which comprises, in combination with an effective amount of a bioactive agent, a matrix comprising a polymer and a targeting ligand, and (ii) administering to a patient said pharmaceutical composition, wherein said targeting ligand is associated covalently with said polymer and said bioactive agent is associated non-covalently with said polymer, and wherein said bioactive agent is substantially homogeneously dispersed throughout said matrix.

100. A method for treating cancer comprising administering to a patient a pharmaceutical composition comprising, in combination with an effective amount of an anticancer agent, a matrix which comprises a polymer and a targeting ligand, wherein said targeting ligand is covalently associated with said polymer and said anticancer agent is associated non-covalently with said polymer, and wherein said anticancer agent is substantially homogeneously dispersed throughout said matrix.
